(19) United States
(12) Patent Application Publication
(10) Pub. No.: US 2021/0355219 A1
(43) Pub. Date: Nov. 18, 2021

(54) CONDITIONALLY ACTIVATED TARGET-BINDING MOLECULES

(71) Applicant: Harpoon Therapeutics, Inc., South San Francisco, CA (US)

(72) Inventors: Shuoyen Jack LIN, San Bruno, CA (US); Holger WESCHE, San Francisco, CA (US); Kathryn KWANT, San Bruno, CA (US)

(21) Appl. No.: 17/276,796

(22) PCT Filed: Sep. 20, 2019
(86) PCT No.: PCT/US19/52270

§ 371 Date: Mar. 16, 2021

Related U.S. Application Data
(60) Provisional application No. 62/734,940, filed on Sep. 21, 2018.

(51) Int. Cl. C07K 16/28; A61P 35/00; C07K 16/18; C07K 16/30

(52) U.S. Cl. CPC C07K 16/2818; A61P 35/00; C07K 16/2827; C07K 16/2863; A61K 2039/505; C07K 16/2878; C07K 16/3069; C07K 2317/31; C07K 16/18

(57) ABSTRACT

Disclosed herein are conditionally active multivalent target-binding proteins which comprise two binding moieties, two linkers, two target antigen binding domains, and a constant domain. Each binding moiety comprises non-CDR loops for masking the binding of a target antigen binding domain to its target and CDRs for binding a further target. The multivalent target-binding proteins are activated upon cleavage of the cleavable linkers. Also disclosed are pro immune modulating molecules comprising dual binding moieties comprising non-CDR loops and cleavable linkers, that prohibit the binding of a target binding domain to their targets (e.g., binding of an antibody to an immune modulatory target is masked by the dual binding moieties). The dual binding moieties further have specificity for a bulk serum protein or a dual binding moiety target (e.g., a tumor antigen, an immune modulatory protein). Pharmaceutical compositions comprising the binding proteins disclosed herein and methods of using such formulations are further provided.

### CROSS-REFERENCE

This application claims the benefit of U.S. Provisional Application No. 62/734,940 filed on Sep. 21, 2018 which is incorporated by reference herein in its entirety.

### INCORPORATION BY REFERENCE

All publications, patents, and patent applications mentioned in this specification are herein incorporated by reference to the same extent as if each individual publication, patent, or patent application was specifically and individually indicated to be incorporated by reference, and as if set forth in their entireties.

### BACKGROUND OF THE INVENTION

There is a need for conditionally activated drugs for improved targeting and delivery, in particular for use in diagnosis and treatment of cancers.

### SUMMARY OF THE INVENTION

The present disclosure provides activatable binding proteins comprising target binding domains that are masked from binding their targets by dual binding moieties. As used herein, “dual binding moieties” or “binding moiety comprising a non-CDR loop,” or “binding moiety,” refer to binding moieties that comprise a cleavable linker and binding specificity for at least two targets, the first binding specificity being for a bulk serum protein or for a target that is not a bulk serum protein (e.g., a tumor antigen, an immune modulator protein, an immune cell), and the second binding specificity being for a target binding domain (e.g., an antibody or an antigen binding fragment thereof) that recognizes an antibody target (e.g., a tumor antigen, an immune modulator protein, an immune cell). The activatable binding proteins are able to switch between an inactive (also referred to herein as “pro” state) and an active state, by cleavage of the cleavable linker (which can be cleaved in a specific environment, such as in presence of a tumor specific protease). In some embodiments, an activatable binding protein of this disclosure is a pro immune modulating molecule. In some cases, a pro immune modulating molecule comprises an antibody as the target binding domain that is masked from binding the antibody target in the pro state of the molecule, the target binding domain being an immune modulating molecule (e.g., an antibody that recognizes an immune modulator protein). The activation event, whereby the cleavable linker is cleaved to activate the “pro” form of the molecule, leads to release of the active drug (e.g., the active drug in some cases is an antibody that is able to bind an immune modulatory protein, which is the antibody target, upon activation). The activation of the pro immune modulating molecules are in some instances linked to alteration of half-life of the same, for example, by introducing mutations in the FcRn binding regions of the target binding protein. In some cases, the FcRn mutations in the target binding protein can reduce binding to FcRn and shorten the half-life of the active drug. In other embodiments, the activation of the pro immune modulating molecule is not linked to alteration of half-life of the active drug, for instance, in some examples the pro immune modulating molecule and the active drug have no half-life differential.

It is contemplated that conditional activation and release of such target binding domains, such as target binding domains that recognize an immune modulator protein can lead to reduction in adverse effects associated with systemic immune modulation. For instance, activation a drug (such as an antibody) in a tumor selective leads to reduced toxicity in some cases.

One embodiment provides a pro immune modulating molecule comprising a first dual binding moiety and a second dual binding moiety and an antibody, wherein each dual binding moiety comprises a cleavable linker, a non-CDR loop, and CDRs for binding a bulk serum protein or a dual binding moiety target, wherein the antibody comprises:
a first light chain polypeptide and a second light chain polypeptide each comprising a light chain variable domain (VL) and a light chain constant domain (CL domain);
a first heavy chain polypeptide and a second heavy chain polypeptide each comprising a variable heavy chain domain (VH), a heavy chain constant region domain (CH1), and constant region domains CH2 and CH3, and wherein the first dual binding moiety and the second dual binding moiety are capable of masking the binding of the antibody to an antibody target, and wherein upon cleavage of the linkers in the first dual binding moiety and the second dual binding moiety the molecule is activated and the antibody is able to bind the antibody target.

In some embodiments, the first dual binding moiety is connected, via its linker, to the N-terminus of the first light chain polypeptide and the second dual binding moiety is connected, via its linker, to the N-terminus of the second light chain polypeptide. In some embodiments, the first dual binding moiety is connected, via its linker, to the N-terminus of the first heavy chain polypeptide and the second dual binding moiety is connected, via its linker, to the N-terminus of the second heavy chain polypeptide. In some embodiments, the non-CDR loops of the first dual binding moiety and the second dual binding moiety provide binding sites that enable binding of the first dual binding moiety and the second dual binding moiety to the antibody. In some embodiments, the first dual binding moiety and the second dual binding moiety each comprises at least three CDRs (CDR1, CDR2, CDR3) for binding a bulk serum protein. In some embodiments, the first dual binding moiety and the second dual binding moiety each comprises at least three CDRs (CDR1, CDR2, CDR3) for binding the dual binding moiety target. In some embodiments, the non-CDR loop comprises at least one of: an AB loop, a C″D loop, an EF loop, and a CC′ loop and the binding site specific for the antibody is provided by one or more of the AB loop, the C″D loop, the EF loop, and the CC′ loop. In some embodiments, the binding site specific for the antibody is provided by the CC′ loop. In some embodiments, the antibody target comprises a tumor antigen, an immune modulatory protein, an immune cell, or a T cell. In some embodiments, the bulk serum protein is at least one of: albumin, transferrin, IgG1, IgG2, IgG4, IgG3, IgA monomer, Factor XIII, Fibrinogen, IgE, and pentameric IgM, or any combinations thereof. In some embodiments, the bulk serum protein is human serum albumin (ALB) and the antibody target comprises the tumor antigen, the immune modulatory protein, the immune cell, or the T cell. In some embodiments, the dual binding moiety target comprises a tumor antigen, an immune modulatory protein, an immune cell, or a T cell. In some embodiments, the antibody target and the dual binding moiety target are different.

In some embodiments, the antibody target is the tumor antigen and wherein the tumor antigen is selected from the group consisting of EGFR, PSMA, EpCAM, BCMA, 5T4, AFP, Axl, B7-H3, Cadherin-6, CAIX, CD117, CD123, CD138, CD166, CD19, CD20, CD205, CD22, CD30, CD33, CD352, CD37, CD38, CD44, CD52, CD56, CD70, CD71, CD74, CD79b, CEACAM5, c-MET, DLL3, EphA2, FAP, FGFR2, FGFR3, glypican-3, FLT-3, FOLR1, gpNMB, HER2, HPV-16 E6, HPV-16 E7, ITGA3, SLC39A6, Mesothelin, Muc1, _k Muc16, NaPi2b, Nectin-4, P-cadherin, Prolactin R, PSCA, PTK7, ROR1, SLC44A4, SLTRK5, SLTRK6, STEAP1, TIM1, Trop2, and WT1.

In some embodiments, the dual binding moiety target is the tumor antigen and wherein the tumor antigen is selected from the group consisting of EGFR, PSMA, EpCAM, BCMA, 5T4, AFP, Axl, B7-H3, Cadherin-6, CAIX, CD117, CD123, CD138, CD166, CD19, CD20, CD205, CD22, CD30, CD33, CD352, CD37, CD38, CD44, CD52, CD56, CD70, CD71, CD74, CD79b, CEACAM5, c-MET, DLL3, EphA2, FAP, FGFR2, FGFR3, glypican-3, FLT-3, FOLR1, gpNMB, HER2, HPV-16 E6, HPV-16 E7, ITGA3, SLC39A6, Mesothelin, Muc1, Muc16, NaPi2b, Nectin-4, P-cadherin, Prolactin R, PSCA, PTK7, ROR1, SLC44A4, SLTRK5, SLTRK6, STEAP1, TIM1, Trop2, and WT1.

In some embodiments, the antibody target is the immune modulatory protein. In some embodiments, the immune modulatory protein is selected from the group consisting of CTLA-4, CD27, CD137, 2B4, TIGIT, CD155, ICOS, HVEM, CD40L, LIGHT, TIM-1, OX40, DNAM-1, PD-L1, PD1, PD-L2, CD8, CD40, CEACAM1, CD48, CD70, A2AR, CD39, CD73, B7-H3, B7-H4, BTLA, IDOL IDO2, TDO, KIR, LAG-3, TIM-3, VISTA, IL6-R, IL-6, TNFα, CD19, CD20, CD22, CD52, integrin a4, integrin a4b7, CD11a, CTLA4-Ig fusion, IL-17, IL12/23, IL12, IL23, and TGF-beta. In some embodiments, the dual binding moiety target is the immune modulatory protein. In some embodiments, the immune modulatory protein is selected from the group consisting of CTLA-4, CD27, CD137, 2B4, TIGIT, CD155, ICOS, HVEM, CD40L, LIGHT, TIM-1, OX40, DNAM-1, PD-L1, PD1, PD-L2, CD8, CD40, CEACAM1, CD48, CD70, A2AR, CD39, CD73, B7-H3, B7-H4, BTLA, IDOL IDO2, TDO, KIR, LAG-3, TIM-3, VISTA, IL6-R, IL-6, TNFα, CD19, CD20, CD22, CD52, integrin a4, integrin a4b7, CD11a, CTLA4-Ig fusion, IL-17, IL12/23, IL12, IL23, and TGF-beta. In some embodiments, the antibody target and the dual binding moiety target are independently the immune cell. In some embodiments, the antibody target and the dual binding moiety target are independently the T cell. In some embodiments, the antibody target and the dual binding moiety target are independently CD3. In some embodiments, the first dual binding moiety and the second dual binding moiety independently comprises a sequence selected from the group consisting of SEQ ID Nos. 48-60 and 63-71. In some embodiments, the antibody target is CTLA-4 and the dual binding moiety target is PSMA. In some embodiments, the light chain polypeptides of the antibody comprise the sequence of SEQ ID No. 78 and the heavy chain polypeptides of the antibody comprise the sequence of SEQ ID No. 79, and wherein the first dual binding moiety and the second dual binding moiety each comprises the sequence of SEQ ID No. 95 or SEQ ID No. 96. In some embodiments, the antibody target is CD40 and the dual binding moiety target is PSMA. In some embodiments, the light chain polypeptides of the antibody comprise the sequence of SEQ ID No. 72 and the heavy chain polypeptides of the antibody comprise the sequence of SEQ ID No. 73, SEQ ID No. 74, SEQ ID No. 75, SEQ ID No. 76, or SEQ ID No. 77, and wherein the first dual binding moiety and the second dual binding moiety each comprises the sequence of SEQ ID No. 97. In some embodiments, the first target is CD40 and the second target is PD1. In some embodiments, the light chain polypeptides of the antibody comprise the sequence of SEQ ID No. 72 and the heavy chain polypeptides of the antibody comprise the sequence of SEQ ID No. 73, SEQ ID No. 74, SEQ ID No. 75, SEQ ID No. 76, or SEQ ID No. 77, and wherein the first dual binding moiety and the second dual binding moiety each comprises the sequence of SEQ ID No. 99. In some embodiments, the antibody target is CTLA-4 and the dual binding moiety target is PD1. In some embodiments, the light chain polypeptides of the antibody comprise the sequence of SEQ ID No. 78 and the heavy chain polypeptides of the antibody comprise the sequence of SEQ ID No. 79, and wherein the first dual binding moiety and the second dual binding moiety each comprises the sequence of SEQ ID No. 98. In some embodiments, the cleavable linker of each dual binding moiety comprises the sequence of SEQ ID No. 100, 101, or 102. In some embodiments, the antibody target is CTLA4 or CD40 and the first dual binding moiety and the second dual binding moiety each comprises CDRs for binding the bulk serum protein. In some embodiments, the antibody target is CTLA4 and the light chain polypeptides of the antibody comprise the sequence of SEQ ID No. 78 and the heavy chain polypeptides of the antibody comprise the sequence of SEQ ID No. 79, and the first and the second dual binding moieties each comprises a sequence selected from SEQ ID Nos. 63-71. In some embodiments, the antibody target is CD40 the light chain polypeptides of the antibody comprise the sequence of SEQ ID No. 72 and the heavy chain polypeptides of the antibody comprise the sequence of SEQ ID No. 73, SEQ ID No. 74, SEQ ID No. 75, SEQ ID No. 76, or SEQ ID No. 77, and the first and the second dual binding moieties each comprises a sequence selected from SEQ ID Nos. 48-60. In some embodiments, the pro immune modulating molecule comprises at least one of:
(a) the CH2 domain of at least one of the first heavy chain polypeptide and the second heavy chain polypeptide comprises one or more mutations that reduce FcRn binding; and
(b) the CH3 domain of at least one of the first heavy chain polypeptide and the second heavy chain polypeptide comprises one or more mutations that reduce FcRn binding,
   wherein upon cleavage of the linkers of the first dual binding moiety and the second dual binding moiety the molecule is activated and the antibody is able to bind the antibody target, and wherein the activated molecule has a shorter half-life compared to an otherwise identical activated molecule that does not have (a) or (b). In some embodiments, at least one of the first heavy chain polypeptide and the second heavy chain polypeptide comprises a 310A mutation at an amino acid position corresponding to H310 in human IgG1 or IgG2. In some embodiments, at least one of the first heavy chain polypeptide and the second heavy chain polypeptide comprises a 310A mutation at an amino acid position corresponding to H310 in human IgG1 or IgG2 and a 435A mutation at an amino acid position corresponding to H435 in human IgG1 or IgG2. In some embodiments, the cleavable linker of the first dual binding moiety and the second dual binding moiety each comprises a protease cleavage site. In some embodiments, the protease cleavage site is recognized by one of a serine protease, a cysteine protease, an aspartate protease, a threonine protease, a glutamic acid protease, a metalloproteinase, a gelatinase, and a asparagine peptide lyase. In some embodiments, the protease cleavage site is recognized by one of a Cathepsin B, a Cathepsin C, a Cathepsin D, a Cathepsin E, a Cathepsin K, a Cathepsin L, a kallikrein, a hK1, a hK10, a hK15, a plasmin, a collagenase, a Type IV collagenase, a stromelysin, a Factor Xa, a chymotrypsin-like protease, a trypsin-like protease, a elastase-like protease, a subtilisin-like protease, an actinidain, a bromelain, a calpain, a caspase, a caspase-3, a Mir1-CP, a papain, a HIV-1 protease, a HSV protease, a CMV protease, a chymosin, a renin, a pepsin, a matriptase, a legumain, a plasmepsin, a nepenthesin, a metalloexopeptidase, a metalloendopeptidase, a matrix metalloprotease (MMP), a MMP1, a MMP2, a MMP3, a MMP7, a MMP8, a MMP9, a MMP10, a MMP11, a MMP12, a MMP13, a MMP14, an ADAM10, an ADAM12, an urokinase plasminogen activator (uPA), an enterokinase, a prostate-specific target (PSA, hK3), an interleukin-1β converting enzyme, a thrombin, a FAP (FAP-α), a type II transmembrane serine protease (TTSP), a neutrophil elastase, a cathepsin G, a proteinase 3, a neutrophil serine protease 4, a mast cell chymase, a mast cell tryptase, a dipeptidyl peptidase, and a dipeptidyl peptidase IV (DPPIV/CD26). In some embodiments, the cleavable linker of the first dual binding moiety and the second dual binding moiety each independently comprises the amino acid sequence of SEQ ID No. 100, SEQ ID No. 101, or SEQ ID No. 102.

One embodiment provides a pro immune modulating molecule comprising:
an antibody or an antigen binding fragment thereof that is capable of binding an antibody target; and
   at least one dual binding moiety that is capable of binding a dual binding moiety target, wherein the at least one dual binding moiety comprises a cleavable linker and a non-CDR loop;
      wherein the at least one dual binding moiety is capable of masking the antibody or an antigen binding fragment thereof from binding the antibody target, and wherein upon cleavage of the linker the molecule is activated and the antibody or an antigen binding fragment thereof is able to bind the antibody target . In some embodiments, the non-CDR loop of the at least one dual binding moiety provides a binding site that enables binding of the dual binding moiety to the antibody or an antigen binding fragment thereof. In some embodiments, the at least one dual binding moiety comprises a binding site specific for the dual binding moiety target and wherein the binding site is provided by one or more CDRs. In some embodiments, the antibody target and the dual binding moiety target independently comprises a tumor antigen, an immune modulatory protein, an immune cell, or a T cell. In some embodiments, the antibody target and the dual binding moiety target are different. In some embodiments, the antibody target and the dual binding moiety target independently are the tumor antigen and wherein the tumor antigen is selected from the group consisting of EGFR, PSMA, EpCAM, BCMA, 5T4, AFP, Axl, B7-H3, Cadherin-6, CAIX, CD117, CD123, CD138, CD166, CD19, CD20, CD205, CD22, CD30, CD33, CD352, CD37, CD38, CD44, CD52, CD56, CD70, CD71, CD74, CD79b, CEACAM5, c-MET, DLL3, EphA2, FAP, FGFR2, FGFR3, glypican-3, FLT-3, FOLR1, gpNMB, HER2, HPV-16 E6, HPV-16 E7, ITGA3, SLC39A6, Mesothelin, Muc1, Muc16, NaPi2b, Nectin-4, P-cadherin, Prolactin R, PSCA, PTK7, ROR1, SLC44A4, SLTRK5, SLTRK6, STEAP1, TIM1, Trop2, and WT1. In some embodiments, the antibody target and the dual binding moiety target independently are the immune modulatory protein wherein the immune modulatory protein is selected from the group consisting of CTLA-4, CD27, CD137, 2B4, TIGIT, CD155, ICOS, HVEM, CD40L, LIGHT, TIM-1, OX40, DNAM-1, PD-L1, PD1, PD-L2, CD8, CD40, CEACAM1, CD48, CD70, A2AR, CD39, CD73, B7-H3, B7-H4, BTLA, IDO1, IDO2, TDO, KIR, LAG-3, TIM-3, VISTA, IL6-R, IL-6, TNFα, CD19, CD20, CD22, CD52, integrin a4, integrin a4b7, CD11a, CTLA4-Ig fusion, IL-17, IL12/23, IL12, IL23, and TGF-beta. In some embodiments, the antibody target and the dual binding moiety target independently are the immune cell. In some embodiments, the antibody target and the dual binding moiety target independently are the T cell. In some embodiments, the antibody target and the dual binding moiety target independently are the CD3. In some embodiments, the antibody target is CTLA-4 and the dual binding moiety target is PSMA. In some embodiments, the dual binding moiety comprises the sequence of SEQ ID No. 95 or SEQ ID No. 96. In some embodiments, the antibody target is CD40 and the dual binding moiety target is PSMA. In some embodiments, the dual binding moiety comprises the sequence of SEQ ID No. 97. In some embodiments, the antibody target is CD40 and the dual binding moiety target is PD1. In some embodiments, the dual binding moiety comprises the sequence of SEQ ID No. 99. In some embodiments, the antibody target is CTLA-4 and the dual binding moiety target is PD1. In some embodiments, the dual binding moiety comprises the sequence of SEQ ID No. 98. In some embodiments, the cleavable linker of the at least one dual binding moiety comprises a protease cleavage site. In some embodiments, the protease cleavage site is recognized by one of a serine protease, a cysteine protease, an aspartate protease, a threonine protease, a glutamic acid protease, a metalloproteinase, a gelatinase, and a asparagine peptide lyase. In some embodiments, the protease cleavage site is recognized by one of a Cathepsin B, a Cathepsin C, a Cathepsin D, a Cathepsin E, a Cathepsin K, a Cathepsin L, a kallikrein, a hK1, a hK10, a hK15, a plasmin, a collagenase, a Type IV collagenase, a stromelysin, a Factor Xa, a chymotrypsin-like protease, a trypsin-like protease, a elastase-like protease, a subtilisin-like protease, an actinidain, a bromelain, a calpain, a caspase, a caspase-3, a Mir1-CP, a papain, a HIV-1 protease, a HSV protease, a CMV protease, a chymosin, a renin, a pepsin, a matriptase, a legumain, a plasmepsin, a nepenthesin, a metalloexopeptidase, a metalloendopeptidase, a matrix metalloprotease (MMP), a MMP 1, a MMP2, a MMP3, a MMP7, a MMP8, a MMP9, a MMP10, a MMP11, a MMP12, a MMP13, a MMP14, an ADAM10, an ADAM12, an urokinase plasminogen activator (uPA), an enterokinase, a prostate-specific target (PSA, hK3), an interleukin-1β converting enzyme, a thrombin, a FAP (FAP-α), a type II transmembrane serine protease (TTSP), a neutrophil elastase, a cathepsin G, a proteinase 3, a neutrophil serine protease 4, a mast cell chymase, a mast cell tryptase, a dipeptidyl peptidase, and a dipeptidyl peptidase IV (DPPIV/CD26). In some embodiments, the cleavable linker of the at least one dual binding moiety comprises the amino acid sequence of SEQ ID No. 100 or SEQ ID No. 101.

One embodiment provides a pro immune modulating molecule comprising a sequence selected from the group consisting of SEQ ID Nos. 80-91.

One embodiment provides a pro immune modulating molecule comprising an antibody that is masked from binding its target by a first dual binding moiety and a second dual binding moiety, wherein each dual binding moiety comprises a cleavable linker and a non-CDR loop, wherein the first dual binding moiety is connected to the N-terminus of a first light chain polypeptide of the antibody, via the linker of the first dual binding moiety and the second dual binding moiety is connected to the N-terminus of a second light chain polypeptide of the antibody, via the linker of the second dual binding moiety, and wherein the Fc region of the antibody comprises at least one of:
(a) a 310A mutation at an amino acid position corresponding to H310 in human IgG1 or IgG2;
(b) and a 435A mutation at an amino acid position corresponding to H435 in human IgG1 or IgG2.
   In some embodiments, upon cleavage of the linkers in the first dual binding moiety and the second dual binding moiety the molecule is activated and the antibody is able to bind its target, and wherein the activated molecule has a shorter half-life compared to an otherwise identical activated molecule that does not comprise (a) or (b). In some embodiments are provided a method of treating a disease comprising administering to a subject an effective amount of a pro immune modulating molecule according to any of the above embodiments. In some embodiments, the subject is human. In some embodiments, the disease is a tumorous disease or an inflammatory disease.

Also provided herein in some embodiments are activatable proteins referred as conditionally active multivalent proteins. One embodiment provides a conditionally active multivalent protein comprising:
a first binding moiety comprising a first non-CDR loop and CDRs, a first cleavable linker, and a first target antigen binding domain capable of binding a first target,
a second binding moiety comprising a second non-CDR loop and CDRs, a second cleavable linker, and a second target antigen binding domain capable of binding a second target, and

a Fc domain;

wherein the first binding moiety and the second binding moiety are each capable of masking the binding of the first and the second target antigen binding domains to their respective targets. In some embodiments, the first binding moiety is capable of binding the first target via the CDRs of the first binding moiety and to the first target antigen binding domain via the first non-CDR loop. In some embodiments, the second binding moiety is capable of binding the second target via the CDRs of the second binding moiety and to the second target antigen binding domain via the second non-CDR loop. In some embodiments, the first and the second targets are the same or different. In some embodiments, the first or the second binding moiety is a natural peptide, a synthetic peptide, an engineered scaffold, or an engineered bulk serum protein. In some embodiments, the engineered scaffold comprises a sdAb, a scFv, a Fab, a VHH, a fibronectin type III domain, immunoglobulin-like scaffold, DARPin, cystine knot peptide, lipocalin, three-helix bundle scaffold, or a DNA or RNA aptamer scaffold. In some embodiments, the first or the second non-CDR loop is a non-CDR loop of a variable domain, a constant domain, a C1 set domain, a C2 set domain, an I-domain, or any combinations thereof.

In some embodiments, the first and the second cleavable linkers each comprise a cleavage site. In some embodiments, the cleavage sites are recognized by a protease, are pH sensitive, or are cleaved by chemical degradation. In some embodiments, the first binding moiety is covalently linked to the first target antigen binding domain and the second binding moiety is covalently linked to the second target antigen binding domain. In some embodiments, upon cleavage of the first and the second cleavable linkers, the first and the second target antigen binding domains bind to their respective targets. In some embodiments, the first binding moiety is capable of masking the binding of the first target antigen binding domain to its target via specific intermolecular interactions between the first binding moiety and the first target antigen binding domain. In some embodiments, the second binding moiety is capable of masking the binding of the second target antigen binding domain to its target via specific intermolecular interactions between the second binding moiety and the second target antigen binding domain. In some embodiments, the first or the second target antigen binding domain comprises a sdAb, a scFv, a Fab, or a variable heavy chain domain (VHH). In some embodiments, the first target antigen binding domain comprises a first variable heavy chain domain (VHH1) and the second target antigen binding domain comprises a second variable heavy chain domain (VHH2). In some embodiments, the first target antigen and the second target antigen are the same antigen and wherein the activated protein binds to two molecules of same antigen. In some embodiments, the first target antigen and the second target antigen are the same antigen and wherein the activated protein binds to two epitopes of the same antigen. In some embodiments, the first target antigen and the second target antigen are different antigens. In some embodiments, the first or the second target binding domains binds to a tumor antigen. In some embodiments, the tumor antigen comprises EGFR, PSMA, EpCAM, BCMA, 5T4, AFP, Ax1, B7-H3, Cadherin-6, CAIX, CD117, CD123, CD138, CD166, CD19, CD20, CD205, CD22, CD30, CD33, CD352, CD37, CD38, CD44, CD52, CD56, CD70, CD71, CD74, CD79b, CEACAM5, c-MET, DLL3, EphA2, FAP, FGFR2, FGFR3, glypican-3, FLT-3, FOLR1, gpNMB, HER2, HPV-16 E6, HPV-16 E7, ITGA3, SLC39A6, Mesothelin, Muc1, Muc16, NaPi2b, Nectin-4, P-cadherin, Prolactin R, PSCA, PTK7, ROR1, SLC44A4, SLTRK5, SLTRK6, STEAP1, TIM1, Trop2, or WT1. In some embodiments, the first or the second target comprises an immune modulatory protein. In some embodiments, the immune modulatory protein comprises CD27, CD137, 2B4, TIGIT, CD155, ICOS, HVEM, CD40L, LIGHT, TIM-1, OX40, DNAM-1, PD-L1, PD1, PD-L2, CTLA-4, CD8, CD40, CEACAM1, CD48, CD70, A2AR, CD39, CD73, B7-H3, B7-H4, BTLA, IDO1, IDO2, TDO, KIR, LAG-3, TIM-3, or VISTA. In some embodiments, the first or the second target antigen binding domain binds to an immune cell. In some embodiments, the first or the second target antigen binding domain binds to a T-cell. In some embodiments, the first or the second target antigen binding domain binds to CD3.

In some embodiments, the first and the second cleavable linkers each comprise protease cleavage sites. In some embodiments, the protease cleavage site is recognized by one of a serine protease, a cysteine protease, an aspartate protease, a threonine protease, a glutamic acid protease, a metalloproteinase, a gelatinase, and a asparagine peptide lyase. In some embodiments, the protease cleavage site is recognized by one of a Cathepsin B, a Cathepsin C, a Cathepsin D, a Cathepsin E, a Cathepsin K, a Cathepsin L, a kallikrein, a hK1, a hK10, a hK15, a plasmin, a collagenase, a Type IV collagenase, a stromelysin, a Factor Xa, a chymotrypsin-like protease, a trypsin-like protease, a elastase-like protease, a subtilisin-like protease, an actinidain, a bromelain, a calpain, a caspase, a caspase-3, a Mir1-CP, a papain, a HIV-1 protease, a HSV protease, a CMV protease, a chymosin, a renin, a pepsin, a matriptase, a legumain, a plasmepsin, a nepenthesin, a metalloexopeptidase, a metalloendopeptidase, a matrix metalloprotease (MMP), a MMP1, a MMP2, a MMP3, a MMP8, a MMP9, a MMP10, a MMP11, a MMP12, a MMP13, a MMP14, an ADAM10, an ADAM12, an urokinase plasminogen activator (uPA), an enterokinase, a prostate-specific target (PSA, hK3), an interleukin-1β converting enzyme, a thrombin, a FAP (FAP-α), a type II transmembrane serine protease (TTSP), a neutrophil elastase, a cathepsin G, a proteinase 3, a neutrophil serine protease 4, a mast cell chymase, a mast cell tryptase, a dipeptidyl peptidase, and a dipeptidyl peptidase IV (DPPIV/CD26). In some embodiments, the Fc domain is capable of extending the half-life of the protein. In some embodiments, the Fc domain comprises an antibody, a sdAb-Fc, or a scFv-Fc. In some embodiments, the conditionally active multivalent protein further comprises a first target bound to the first binding moiety and a second target bound to the second binding moiety, wherein the first and the second targets provide the multivalent protein with a systemic pharmacological activity, and wherein upon cleavage of the first and the second cleavable linkers the multivalent protein is activated by separation of the first and the second binding moiety and the first and the second targets, from the first and the second target antigen binding domains, respectively, and the binding protein is thereby activated for local binding with first and second target antigens. In some embodiments, the cleavage of the first and the second cleavable linkers are in a tumor microenvironment. In some embodiments, the first and the second targets are PD-L1, and wherein the first and the second target antigens are CTLA4.

One embodiment provides a conditionally active multivalent protein, comprising:
a first binding moiety bound to a first target antigen binding domain by a first non-CDR loop, bound to a first target domain by CDRs, wherein the first binding moiety and the first target antigen binding domains are connected by a first cleavable linker
a second binding moiety bound to a first target antigen binding domain by a second non-CDR loop, bound to a second target domain by CDRs, wherein the second binding moiety and the second target antigen binding domains are connected by a second cleavable linker; and
an Fc domain;;
   wherein the first and the second target antigen binding domains are capable of binding to a first target antigen and a second target antigen, respectively,
   wherein the multivalent protein has a systemic pharmacological activity prior to its activation by cleavage of the first and the second cleavable linkers, and wherein upon activation the first and the second targets and the first and the second binding moieties are separated from the first and the second target antigen binding domains, and wherein the multivalent protein, in its activated state, binds locally with the first and second target antigens. In some embodiments, the cleavage of the first and the second cleavable linkers is in a tumor microenvironment. In some embodiments, the first and the second targets are PD-L1, and wherein the first and the second target antigens are CTLA4.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which.

FIG. 1 illustrates a variable domain of an immunoglobulin molecule, comprising complementarity determining regions (CDR1, CDR2, and CDR3), and non-CDR loops connecting the beta strand (AB, CC′, C″ D, EF, and DE).

FIG. 2 shows an embodiment of the conditionally active multivalent target-binding protein of the present disclosure.

FIG. 3 shows the activation of an exemplary conditionally active multivalent target-binding protein of the present disclosure.

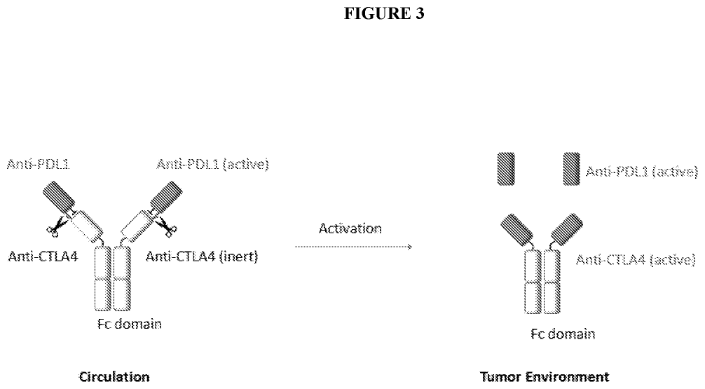

FIG. 4 shows a schematic representation of Pro Immune Modulating Molecules. A) In this exemplary PIMMS illustration is shown a dual binding moiety (labeled as anti-target 1 in the figure) capable of binding a target and containing non-CDR loop mutations to mask the activity of an antibody (labeled as anti-Target 2 in the figure). The dual binding moiety is connected to the antibody through a protease cleavable linker. B) In this exemplary PIMMS illustration is shown a dual binding moiety (labeled as aAlb) which is an anti-Albumin domain that is capable of binding an albumin and containing non-CDR loop mutations to mask the activity of anti-Target antibody (labeled as atarget). The dual binding moiety is connected to the antibody through a protease cleavable linker. In addition, in the illustrated example PIMMS, the constant region of the antibody contains mutations to reduce FcRn affinity, thereby shortening the half-life of the protease activated molecule.

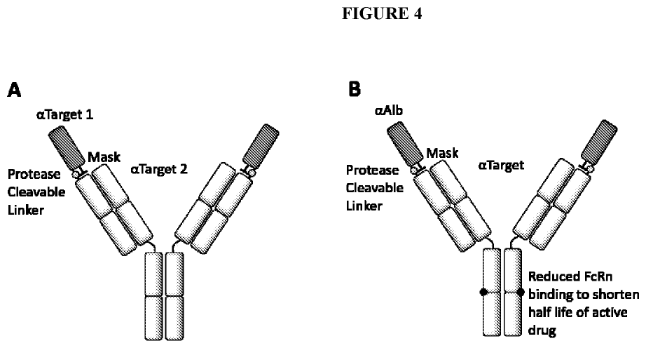

FIG. 5 illustrates binding of exemplary masked anti-CD40 and control unmasked anti-CD40 proteins to immobilized human CD40.

FIG. 6 illustrates B cell activation by masked (anti-Alb mask070) and activated anti-CD40 proteins measured by percentage of CD86 positive cells.

FIG. 7 illustrates binding of exemplary masked anti-CTLA4 and control unmasked anti-CTLA4 proteins binding immobilized human CTLA4.

FIG. 8 shows binding of anti-Alb, anti-PSMA, and anti-PD1 masked anti-CTLA4 proteins to human albumin (A, B), human PSMA (C, D), and human PD1 (E). The single domain antibodies contain mask071 (A, C) or mask078 (B, D, E).

FIG. 9 illustrates ADCC (antibody dependent cell cytotoxic) activity of exemplary anti-Alb masked and activated anti-CTLA4 proteins.

FIG. 10 shows results of a CTLA4 blockade bioassay.

### DETAILED DESCRIPTION OF THE INVENTION

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby

### Certain Definitions

The terminology used herein is for the purpose of describing particular cases only and is not intended to be limiting. As used herein, the singular forms “a”, “an” and “the” are intended to include the plural forms as well, unless the context clearly indicates otherwise. Furthermore, to the extent that the terms “including”, “includes”, “having”, “has”, “with”, or variants thereof are used in either the detailed description and/or the claims, such terms are intended to be inclusive in a manner similar to the term “comprising.”

The term “about” or “approximately” means within an acceptable error range for the particular value as determined by one of ordinary skill in the art, which will depend in part on how the value is measured or determined, e.g., the limitations of the measurement system. For example, “about” can mean within 1 or more than 1 standard deviation, per the practice in the given value. Where particular values are described in the application and claims, unless otherwise stated, the term “about” should be assumed to mean an acceptable error range for the particular value.

The terms “individual,” “patient,” or “subject” are used interchangeably. None of the terms require or are limited to situation characterized by the supervision (e.g. constant or intermittent) of a health care worker (e.g. a doctor, a registered nurse, a nurse practitioner, a physician's assistant, an orderly, or a hospice worker).

The term “Framework” or “FR” residues (or regions) refer to variable domain residues other than the CDR or hypervariable region residues as herein defined. A “human consensus framework” is a framework which represents the most commonly occurring amino acid residue in a selection of human immunoglobulin VL or VH framework sequences.

As used herein, “Variable region” or “variable domain” refers to the fact that certain portions of the variable domains differ extensively in sequence among antibodies and are used in the binding and specificity of each particular antibody for its particular antigen. However, the variability is not evenly distributed throughout the variable domains of antibodies. It is concentrated in three segments called complementarity-determining regions (CDRs) or hypervariable regions both in the light-chain and the heavy-chain variable domains. The more highly conserved portions of variable domains are called the framework (FR). The variable domains of native heavy and light chains each comprise four FR regions, largely adopting a β-sheet configuration, connected by three CDRs, which form loops connecting, and in some cases forming part of, the βsheet structure. The CDRs in each chain are held together in close proximity by the FR regions and, with the CDRs from the other chain, contribute to the formation of the antigen-binding site of antibodies (see Kabat et al., Sequences of Proteins of Immunological Interest, Fifth Edition, National Institute of Health, Bethesda, Md. (1991)). The constant domains are not involved directly in binding an antibody to an antigen, but exhibit various effector functions, such as participation of the antibody in antibody-dependent cellular toxicity. “Variable domain residue numbering as in Kabat” or “amino acid position numbering as in Kabat,” and variations thereof, refers to the numbering system used for heavy chain variable domains or light chain variable domains of the compilation of antibodies in Kabat et al., Sequences of Proteins of Immunological Interest, 5th Ed. Public Health Service, National Institutes of Health, Bethesda, Md. (1991). Using this numbering system, the actual linear amino acid sequence may contain fewer or additional amino acids corresponding to a shortening of, or insertion into, a FR or CDR of the variable domain. For example, a heavy chain variable domain may include a single amino acid insert (residue 52a according to Kabat) after residue 52 of H2 and inserted residues (e.g., residues 82a, 82b, and 82c, etc. according to Kabat) after heavy chain FR residue 82. The Kabat numbering of residues may be determined for a given antibody by alignment at regions of homology of the sequence of the antibody with a “standard” Kabat numbered sequence. It is not intended that CDRs of the present disclosure necessarily correspond to the Kabat numbering convention.

A “single domain antibody” or “sdAb” as used herein refers to a type of single chain antibody comprising a variable region (VHH) of a heavy chain of a human antibody. SdAbs are antibody fragments consisting of a single monomeric variable antibody domain. They are derived, for example, from heavy chain antibodies derived from humans, which consist only of two antibody heavy chains, with no light chain. With a molecular weight of only 12-15 kD, sdAbs are much smaller than monoclonal antibodies (mAbs), e.g., IgG antibodies (150-160 kD), which have two heavy protein chains and two light chains.

A “single chain Fv” or “scFv”, as used herein, refers to a binding protein in which the variable domains of the heavy chain and of the light chain of a traditional two chain antibody are joined to form one chain. Typically, a linker peptide is inserted between the two chains to allow for proper folding and creation of an active binding site.

A “cleavage site for a protease,” as meant herein, is an amino acid sequence that can be cleaved by a protease, such as, for example, a matrix metalloproteinase or a furin. Examples of such sites include Gly-Pro-Leu-Gly-Ile-Ala-Gly-Gln or Ala-Val-Arg-Trp-Leu-Leu-Thr-Ala, which can be cleaved by metalloproteinases, and Arg-Arg-Arg-Arg-Arg-Arg, which is cleaved by a furin. In therapeutic applications, the protease cleavage site can be cleaved by a protease that is produced by target cells, for example cancer cells or infected cells, or pathogens.

As used herein, “elimination half-time” is used in its ordinary sense, as is described in Goodman and Gillman's The Pharmaceutical Basis of Therapeutics 21-25 (Alfred Goodman Gilman, Louis S. Goodman, and Alfred Gilman, eds., 6th ed. 1980). Briefly, the term is meant to encompass a quantitative measure of the time course of drug elimination. The elimination of most drugs is exponential (i.e., follows first-order kinetics), since drug concentrations usually do not approach those required for saturation of the elimination process. The rate of an exponential process may be expressed by its rate constant, k, which expresses the fractional change per unit of time, or by its half-time, t_{1/2}the time required for 50% completion of the process. The units of these two constants are time⁻¹ and time, respectively. A first-order rate constant and the half-time of the reaction are simply related (k×t_{1/2}=0.693) and may be interchanged accordingly. Since first-order elimination kinetics dictates that a constant fraction of drug is lost per unit time, a plot of the log of drug concentration versus time is linear at all times following the initial distribution phase (i.e. after drug absorption and distribution are complete). The half-time for drug elimination can be accurately determined from such a graph.

As used herein, “multivalent” refers to the ability of the conditionally active target-binding proteins described herein to bind to two antigen molecules that are same or different. Thus, in certain cases the conditionally active multivalent target binding proteins are bispecific.

Bispecific antibodies simultaneously bind to two different target antigens within a single molecule. Such bispecific antibodies have potential clinical benefits for the treatment of complicated diseases, such as tumors and immune disorders. A heterodimeric Fc-based IgG-like format is attractive because it can be designed as close as possible to the natural IgG architecture such that it possesses desirable physicochemical properties, such as high stability, large-scale manufacturing capability, and low immunogenicity, in addition to the natural IgG-like properties of a long serum half-life and immune cell-recruiting effector functions. In some cases when a normal or healthy tissue expresses the same antigens as a tumor cell, toxicities are possible due to off-tumor antigen binding. In some embodiments, the multivalent proteins disclosed herein are advantageously activated in a tumor tissue specific manner, upon exposure to proteases prevalent in tumor microenvironment. The inhibitory domains obstruct the binding domains and accordingly prevent binding of the proteins to their targets until the inhibitory domains are cleaved by proteases. Described herein are conditionally active multivalent target-binding protein, pharmaceutical compositions as well as nucleic acids, recombinant expression vectors, and host cells for making such conditionally active. Also provided are methods of using the disclosed conditionally active binding proteins in the prevention, and/or treatment of diseases, conditions and disorders. The conditionally active multivalent target-binding proteins of this disclosure are heteromultimeric proteins, which are capable of binding to multiple targets. The disclosure presents a novel bispecific approach to combine oncology with autoimmune/anti-inflammatory drugs

### Binding Moiety for Masking of Target Binding Domains

In some aspect, the disclosure provides a pro immune modulating molecule, comprising a dual binding moiety that comprises a non-CDR loop and a cleavable linker, which is capable of masking a target binding domain (such as an antibody or an antigen binding fragment thereof) from binding its target. Exemplary pro immune modulating molecules comprise a full length antibody with two heavy chain polypeptides and two light chain polypeptides, wherein the antibody is specific for an antibody target, and the dual binding moieties that are attached to the light chain polypeptides or heavy chain polypeptides, for instance via cleavable linkers. For example, each light chain polypeptide of the antibody is connected to one dual binding moiety, in some embodiments, where the target binding domain is an antibody.

The non-CDR loop within the dual binding moieties, in some embodiments, provides a binding site specific for the target binding domain. In some embodiments, in the pro form of the pro immune modulating molecule, the dual binding moiety specifically interacts with the target binding domain and prevents it from binding its target. The pro form of the molecule is activated by cleavage of the cleavable linker(s) that attach the dual binding moiety and the target binding domain. The target binding domain (e.g., an antibody) is released as an active drug when the dual binding moiety is removed from the pro immune modulating molecule. The active drug is cleared from circulation rapidly or after a period of time, depending on its half-life. In some instances, the target binding domain is an antibody comprising mutations in the Fc region that reduce binding of the antibody to FcRn and shorten the half-life of the antibody, which is the active drug released by activation of the pro immune modulating molecule.

The dual binding moieties further comprise CDRs that provide specificity for binding either a bulk serum protein or a dual binding moiety target, which is the same or different as the target recognized by the masked target binding domain (the antibody target).

The pro immune modulating molecules of this disclosure are in some embodiments, suitable for delivery of active drugs that have potential for severe adverse effects so that drugs are not activated non-specifically, and in some examples, modifications may be made to remove drugs with severe adverse effects rapidly from circulation. In some instances, the dual binding moiety comprises a binding specificity for a bulk serum protein, such as albumin, and the target binding domain is an antibody comprising mutations in the Fc region that reduce binding of the Fc region to FcRn and shorten the half-life of the active drug. In some instances, the dual binding moiety comprises a binding specificity for a dual binding target, and the target binding domain is an antibody that does not comprise mutations in the Fc region for reducing binding to FcRn and shortening the half-life of the active drug. Such a pro immune modulating molecule is in some cases suitable for delivery of drugs that do not need to be rapidly cleared from circulation. In some embodiments, the dual binding moiety comprises a binding specificity for a dual binding target and the target binding domain is an antibody that comprises mutations in the Fc region that reduce binding of the Fc region to FcRn and shorten the half-life of the active drug. In some embodiments, the dual binding moiety comprises a binding specificity for albumin and the target binding domain is an antibody that does not comprise mutations in the Fc region for reducing binding to FcRn and shortening the half-life of the active drug.

In some cases, the antibody target, the dual binding moiety target, or both, are targets that are expressed on the surface of a diseased cell or tissue, for example a tumor or a cancer cell. Non-limiting examples include but are not limited to IL-1 , IL-1 receptor, IL-4, IL-4 receptor, VEGF, VEGF receptor, RSV, NGF, NGF receptor, programmed cell death protein-1 (PD1), programmed cell death protein ligand-1 (PD-L1), PD-L2, PDGF, PDGF receptor, angiopoietin-2 (Ang2), Ang2 receptor, myostatin (GDF8), GDF8 receptor, CD3, CD20, EGFR, MSLN, PSMA, DLL3, BCMA, EpCAM, HER-2, HER-3, c-Met, FoIR, CD38, CEA, 5T4, AFP, B7-H3, Cadherin-6, CAIX, CD117, CD123, CD138, CD166, CD19, CD20, CD205, CD22, CD30, CD33, CD352, CD37, CD44, CD52, CD56, CD70, CD71, CD74, CD79b, DLL3, EphA2, FAP, FGFR2, FGFR3, GPC3, gpA33, FLT-3, gpNMB, HPV-16 E6, HPV-16 E7, ITGA2, ITGA3, SLC39A6, MAGE, Muc1, Muc16, NaPi2b, Nectin-4, P-cadherin, NY-ESO-1, PRLR, PSCA, PTK7, ROR1, SLC44A4, SLTRK5, SLTRK6, STEAP1, TIM1, Trop2, and WT1.

Additional non-limiting examples of antibody targets and dual binding moiety targets include but are not limited to immune modulatory proteins, including but not limited to immune modulator proteins, immune stimulatory proteins, immune co-stimulatory proteins, or combinations thereof. Examples of immune modulatory proteins include, but are not limited to: CTLA-4, CD27, CD137, 2B4, TIGIT, CD155, ICOS, HVEM, CD40L, LIGHT, TIM-1, OX40, DNAM-1, PD-L1, PD1, PD-L2, CD8, CD40, CEACAM1, CD48, CD70, A2AR, CD39, CD73, B7-H3, B7-H4, BTLA, IDO1, IDO2, TDO, KIR, LAG-3, TIM-3, VISTA, IL6-R, IL-6, TNFα, CD19, CD20, CD22, CD52, integrin a4, integrin a4b7, CD11a, CTLA4-Ig fusion, IL-17, IL12/23, IL12, IL23, and TGF-beta. The target binding domains (such as antibodies) in some instances are able to inhibit an antibody target, e.g., a target binding moiety is some examples is an anti-CTLA4 antibody which inhibits CTLA4 check point signaling. The target binding domains (such as antibodies) in some instances are able to act as an agonist of an antibody target, e.g., an anti-CD40 antibody which acts as an agonist of CD40.

Numerous studies have indicated that CD40 & CD40L pathway is a major regulating factor of immune responses. Briefly, CD40 & CD40L pathway is believed to affect the immune system in the following exemplary ways: (1) CD40 & CD40 pathway “activates” or “matures” antigen-presenting cells (APCs, mainly macrophages and dendritic cells) to express co-stimulatory molecules including B7 (CD80 and CD86, both ligands for CD28), ICAM-1 (CD54), and CD44. These co-stimulatory signals are needed for T cells to become fully activated, rather than anergic, after T cell receptor (TCR)-stimulation. (2) CD40 & CD40L pathway can induce macrophages and dendritic cells to make interleukin-12 (IL-12), IL-18, and other cytokines. In an immunological response, CD40L/CD154/TNFSF5 is the primary stimulus for IL-12 production (in the absence of microbial invasion). IL-12 and IL-18 stimulate NK cells for interferon-γ (IFN-γ) production. IL-12 causes CD4+ T cells to differentiate into type 1 helper T cells (Th1) that mediate delayed-type hypersensitivity responses. (3) CD40L/CD154/TNFSF5-expressing CD4+ T cells are generally required for the generation of cytotoxic T lymphocytes (CTLs) against tumors and virus-infected cells. As in CD4+ T cell activation, CD40 & CD40L pathway can activate APCs to express the co-stimulatory molecules needed to fully activate or cross-prime CTLs already responding to antigen/MHC class I complexes. (4) CD40 & CD40L pathway may also promote the differentiation of activated B cells and, with few exceptions, is likely required for the class switch from IgM to IgG production. See, e.g., Richard S. Kornbluth et al. The emerging role of CD40 ligand in HIV infection. Journal of Leukocyte Biology. 2000; 68:373-382.

In some examples, the antibody target is PD1. In some examples, the antibody target is CD40. In some examples, the antibody target is CTLA4. In some examples, the dual binding moiety target is PD1. In some examples, the dual binding moiety target is PSMA. In some embodiments, the dual binding moieties recognize a tumor antigen via their CDR loops (e.g., PSMA) and a binding protein that recognizes an immune modulator protein via their non-CDR loops (e.g., an antibody that recognizes an immune checkpoint protein, such as anti-CTLA4 antibody, an anti-CD40 antibody). In some embodiments, the dual binding moieties recognize an immune checkpoint protein via their CDR loops (e.g., PD1) and a protein that recognizes an immune modulatory protein via their non-CDR loops (e.g., the dual binding moiety can interact with an immune checkpoint inhibitory antibody, e.g., an anti-CTLA4 antibody, or an immune agonist antibody, e.g., an anti-CD40 antibody, via the non-CDR loops). In some embodiments, the antibody target is CTLA4 or CD40 and the dual binding moiety comprises a binding site specific for (i) albumin and (ii) an anti-CTLA4 antibody or an anti-CD40 antibody. In some embodiments, the antibody target is anti-TNFα and the dual binding moiety comprises a binding site specific for (i) albumin (via CDR) and (ii) an anti-TNFα antibody (via non-CDR loops). In some embodiments, the antibody target is anti-TNFα and the dual binding moiety comprises a binding site specific for (i) albumin (via CDR) and (ii) an anti-IL6R (anti-IL6 receptor antibody) (via non-CDR loops). In some embodiments, the dual binding moieties recognize TNFα via their CDR loops and an anti-TNFα via their non-CDR loops. In some embodiments, the dual binding moieties recognize TNFα via their CDR loops and an anti-IL6R via their non-CDR loops.

In various embodiments, pro immune modulating molecules achieve a global inhibition of an immune modulator protein when in the global configuration state as well as after activation by cleavage of the cleavable linker (e.g., in cases where the antibody target is an immune modulatory protein, for instance an immune checkpoint protein such as PD1/PD-L1/CTAL4 and the dual binding moiety target is also an immune modulatory protein, same or different from the antibody target). The global configuration state, in some cases, is the state prior to activation where the pro immune modulating molecule is in circulation, the target binding domain being masked from binding its target. The immune modulation, e.g., immune checkpoint inhibition, immune checkpoint agonist activity, or immune agonist activity is achieved both outside and inside tumor. In some cases, the global modulation of an immune modulatory protein is beneficial. Upon activation, the multivalent protein is able to locally inhibit an immune modulatory protein, such as an immune checkpoint protein inside the tumor. Upon activation, the multivalent protein is able to locally cause an agonist effect with respect to an immune modulatory protein inside the tumor, such as, using an immune checkpoint protein agonist that targets proteins such as CD40, CD137. This, in some cases, expands the narrow therapeutic window for existing immune modulatory therapies.

In some embodiments are provided dual binding moieties that comprise a binding site specific for albumin (via its CDRs) and binding site that is specific for an anti-CD40 antibody (via its non-CDR loops). Exemplary sequences for such anti-CD40 specific dual binding moieties are provided in SEQ ID Nos. 48-60, or a sequence that is at least about 75% to about 100% identical to any one of sequences provided in SEQ ID Nos. 48-60, such as at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or at least about 100% . In some embodiments are provided dual binding moieties that comprise a binding site specific for albumin (via its CDRs) and binding site that is specific for an anti-CTLA4 antibody (via its non-CDR loops). Exemplary sequences for such anti-CTLA4 specific dual binding moieties are provided in SEQ ID Nos. 63-71, or a sequence that is at least about 75% to about 100% identical to any one of the sequences provided in SEQ ID Nos. 63-71, such as at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or at least about 100%. In some embodiments are provided dual binding moieties that comprise a binding site specific for PSMA (via its CDRs) and a binding site specific for an anti-CTLA4 (via its non-CDR loops) (e.g., SEQ ID Nos. 95 and 96) and dual binding moieties that comprise a binding site specific for PD1 (via its CDRs) and a binding site specific for an anti-CTLA4 (via its non-CDR loops) (e.g., SEQ ID No. 98). In some embodiments are provided dual binding moieties that comprise a binding site specific for PD-1 (via its CDRs) and a binding site specific for an anti-CD40 (via its non-CDR loops) (e.g., SEQ ID No. 99) and dual binding moieties that comprise a binding site specific for PSMA (via its CDRs) and a binding site specific for an anti-CD40 (via its non-CDR loops) (e.g., SEQ ID No. 97).

In some embodiments, a pro immune modulating molecule comprises a target binding domain that is an antibody, for example, an antibody specific for CD40 or CTLA4. Exemplary sequences for anti-CD40 antibodies that are in some cases part of the pro immune modulating molecules, include, SEQ ID No. 72 as light chain and any one of SEQ ID Nos. 73-77 as heavy chain. Exemplary sequences for anti-CTLA4 antibodies that are in some cases part of the pro immune modulating molecules, include, SEQ ID No. 78 as light chain and SEQ ID No. 79 as heavy chain.

In some embodiments, a pro immune modulating molecule of this disclosure comprises a sequence selected from the group consisting of 80-91, or a sequence that is at least about 75% to about 100% identical to a sequence provided in any one of SEQ ID Nos. 80-91, such as at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or at least about 100%.

In one aspect, the disclosure provides a conditionally active target-binding protein comprising a first binding moiety comprising a first non-CDR loop, a first cleavable linker, and a first target antigen binding domain capable of binding a first target; a second binding moiety comprising a second non-CDR loop, a second cleavable linker, and a second target antigen binding domain capable of binding a second target, and and an Fc domain. The first binding moiety and the second binding moiety are each capable of masking the binding of the first and the second target antigen binding domains to their respective targets. The conditionally active protein is in a global configuration state when first and the second binding moieties are associated with the N-termini of the first and second target antigen binding domain, respectively, such that said binding domains are prevented from binding their targets. In its global configuration state neither the first target antigen binding domain nor the second antigen binding domain is able to bind a target. Upon cleavage of cleavable linker, the conditionally active target-binding protein adopts a local binding active configuration. In the local binding active configuration the first and the second binding domains of the conditionally active multivalent target-binding protein are capable of binding to their target antigen molecules.

In some embodiments, the first binding moiety binds to a target, the second binding moiety binds to a target. In some examples, the first and the second binding moieties bind to the same target. Targets for the first and the second binding moieties, in some cases, are high therapeutic index proteins, or proteins for which systemic pharmacological activity is desirable, and/or proteins which do not require Fc effector functions, e.g., PD-L1.

In some cases, the targets are expressed on the surface of a diseased cell or tissue, for example a tumor or a cancer cell. Targets include but are not limited to IL-1 , IL-1 receptor, IL-4, IL-4 receptor, VEGF, VEGF receptor, RSV, NGF, NGF receptor, programmed cell death protein-1 (PD1), programmed cell death protein ligand-1 (PD-L1), PD-L2, PDGF, PDGF receptor, angiopoietin-2 (Ang2), Ang2 receptor, myostatin (GDF8), GDF8 receptor, CD3, CD20, CTLA4, CD40, EGFR, MSLN, PSMA, DLL3, and BCMA. In some examples, the first binding moiety and the second binding moiety each binds to PD-L1. In some examples, the first binding moiety and the second binding moiety each binds to PD1. In some examples, the first binding moiety and the second binding moiety each binds to PSMA.

In some cases, after activation, the conditionally active multivalent target-binding protein exhibits a local binding or inhibition of a target antigen, such as a tumor antigen. Exemplary target antigens for the first and the second target antigen binding domains include, but are not limited to, EpCAM, EGFR, HER-2, HER-3, c-Met, FoIR, PSMA, CD38, BCMA, and CEA, 5T4, AFP, B7-H3, Cadherin-6, CAIX, CD117, CD123, CD138, CD166, CD19, CD20, CD205, CD22, CD30, CD33, CD352, CD37, CD44, CD52, CD56, CD70, CD71, CD74, CD79b, DLL3, EphA2, FAP, FGFR2, FGFR3, GPC3, gpA33, FLT-3, gpNMB, HPV-16 E6, HPV-16 E7, ITGA2, ITGA3, SLC39A6, MAGE, mesothelin, Muc1, Muc16, NaPi2b, Nectin-4, P-cadherin, NY-ESO-1, PRLR, PSCA, PTK7, ROR1, SLC44A4, SLTRK5, SLTRK6, STEAP1, TIM1, Trop2, and WT1. In some embodiments, a target antigen is an immune modulator protein. Examples of immune modulatory proteins include but are not limited to CTLA-4, CD27, CD137, 2B4, TIGIT, CD155, ICOS, HVEM, CD40L, LIGHT, TIM-1, OX40, DNAM-1, PD-L1, PD1, PD-L2, CD8, CD40, CEACAM1, CD48, CD70, A2AR, CD39, CD73, B7-H3, B7-H4, BTLA, IDO1, IDO2, TDO, KIR, LAG-3, TIM-3, VISTA, IL6-R, IL-6, TNFα, CD19, CD20, CD22, CD52, integrin a4, integrin a4b7, CD11a, CTLA4-Ig fusion, IL-17, IL12/23, IL12, IL23, and TGF-beta. The target antigen binding domains thus are, in some embodiments, immune checkpoint inhibitors (e.g., antibodies that inhibit PD1, PDL1, CTLA4; immune agonists (e.g., antibodies that act as agonists of CD40, CD137 etc.), or immune checkpoint agonists (e.g., antibodies that act as agonists of PD1).

The multivalent target binding proteins, in some embodiments, comprise six binding sites, one each for binding of the first and the second binding moieties to target antigens, e.g., immune modulatory proteins, one each for binding of the first and the second binding moieties to the first and the second target antigen binding domains, and one each for binding of the first and second target antigen binding domains to their targets, e.g., tumor specific antigens, wherein the binding sites for the binding of the first and second target antigen binding domains to their targets are blocked when the protein is in the global configuration state.

Examples of first and second binding moieties include but are not limited to domains from a monoclonal antibody, a polyclonal antibody, a recombinant antibody, a human antibody, a humanized antibody. In some embodiments, the binding moiety is a single chain variable fragment (scFv), single-domain antibody such as a heavy chain variable domain (VH), a light chain variable domain (VL) and a variable domain (VHH) of camelid derived nanobody. In other embodiments, the binding moiety is a non-Ig binding domain, i.e., antibody mimetic, such as anticalins, affilins, affibody molecules, affimers, affitins, alphabodies, avimers, DARPins, fynomers, kunitz domain peptides, and monobodies.

The first and the second binding moiety respectively masks the binding of a target antigen binding domain, and is capable of binding to a target antigen, e.g., an immune modulatory protein. The binding moieties, in certain instances, further comprise a cleavable linker. The cleavable linker, for example, comprises a protease cleavage site or a pH dependent cleavage site. The cleavable linker, in certain instances, is cleaved preferentially in a tumor micro-environment. Thus, in some examples, the binding moieties bound to target antigens, e.g., immune modulatory proteins, connected to the cleavable linkers, and further bound to the first and the second target antigen binding domains, maintains the conditionally active multivalent protein in a global configuration state in circulation until the cleavable linker is cleaved off in a tumor microenvironment. In some embodiments, the half-life of the target antigen binding domain, such as an antibody or an antigen binding fragment thereof, is extended in systemic circulation by using a Fc domain. In some cases, the first and the second binding moieties act as safety switches that keeps the conditionally active multivalent target-binding protein in a global configuration state until it reaches the tumor microenvironment where it is conditionally activated by cleavage of the linker and the first and the second target antigen binding domains are able to bind to their target antigen molecules, e.g., tumor specific antigens.

The safety switch provides several advantages, some examples including (i) expanding the therapeutic window of the conditionally active multivalent target-binding protein, (ii) reducing the probability of rapid clearance of the conditionally active multivalent target-binding protein as a result of, for example, target-mediated clearance, by maintaining it in a global configuration state when it is in systemic circulation, (iii) reducing the concentration of undesirable activated proteins in systemic circulation, thereby minimizing the spread of chemistry, manufacturing, and controls related impurities, e.g., endogenous viruses, host-cell proteins, DNA, leachables, anti-foam, antibiotics, toxins, solvents, heavy metals; (iv) reducing the concentration of undesirable activated proteins in systemic circulation, thereby minimizing the spread of product related impurities, aggregates, breakdown products, product variants due to: oxidation, deamidation, denaturation, loss of C-term Lys in MAbs; (v) preventing aberrant activation of the target antigen binding domain in circulation; (vi) reducing the toxicities associated with the leakage of activated species from diseased tissue or other pathophysiological conditions, e.g., tumors, autoimmune diseases, inflammations, viral infections, tissue remodeling events (such as myocardial infarction, skin wound healing), or external injury (such as X-ray, CT scan, UV exposure); and (vii) reducing non-specific binding of the conditionally active multivalent target-binding protein. Furthermore, post-activation, or in other words post breaking of the safety switch, the conditionally active multivalent target-binding protein is separated from the safety switch, and thus is cleared from circulation. In some embodiments, the half-life of the conditionally active multivalent target-binding protein is not different when it is in the inactive/global configuration state and when it is in an active state and as such no half-life differential is obtained by the masking (e.g., in cases where there are no mutations in the Fc regions of the target binding proteins, mutations that reduce FcRn binding and shorten half-life).

In addition, the binding moiety as described herein, in some cases, is used to generate a “biobetter” version of a biologic. Generally, preparing a biobetter form of a molecule, e.g., an antibody or an antigen binding fragment thereof, involves taking the originator molecule and making specific alterations in it to improve its parameters and thereby make it a more efficacious, less frequently dosed, better targeted, and/or a better tolerated drug. Thus, a first and the second target antigen binding domains masked by the first and the second binding moieties which are bound to target antigens, e.g., immune modulator proteins, and conditionally activated in a tumor microenvironment by cleavage of the cleavable linker, and further being bound to an Fc domain, gives the conditionally active multivalent target-binding protein a significantly longer serum half-life and reduces the likelihood of its undesirable activation in circulation, thereby producing a “biobetter” version of the multivalent target-binding protein. According, in some embodiments, a biobetter version of a multivalent target-binding protein is provided, wherein the biobetter function is attributed to the Fc domain as described herein.

Similarly, in a pro immune modulating molecule containing one or more dual binding moieties bound to an albumin or a dual binding moiety target and linked to a target binding domain, the target binding domain is imparted a significantly longer serum half-life when inactive, the likelihood of its undesirable activation in circulation is reduced, thereby producing a “biobetter” version of the target binding domain.

The binding moieties described herein comprise at least one a non-CDR loop. In some embodiments, a non-CDR loop provides a binding site for binding of the moiety to the first or the second target antigen binding domains of the conditionally active multivalent target-binding protein. In some embodiments, a binding moiety in a conditionally active multivalent target-binding protein masks binding of the first or the second target binding domain to a target antigen, e.g., via specific intermolecular interactions or by steric occlusion. In some embodiments, the binding moieties further comprise complimentary determining regions (CDRs). The dual binding moieties in a pro immune modulating molecule similarly comprises a non-CDR loop that provides a binding site of the moiety to the target binding domain, and mask the binding of the target binding domain to an antibody target. The dual binding moieties further comprise CDRs.

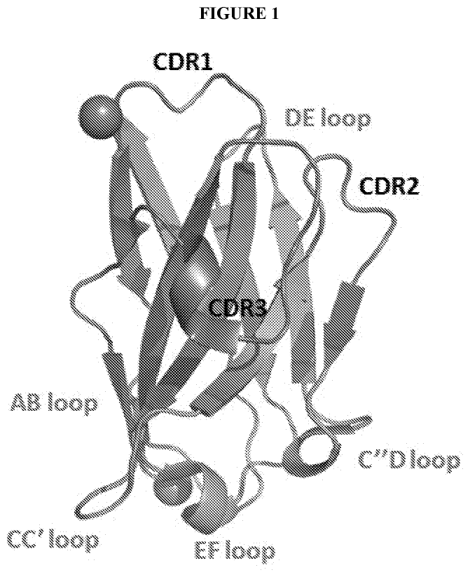

In some instances, the dual binding moiety of a pro immune molecule or the first and second binding moieties of a conditionally active multivalent target-binding protein, are domains derived from an immunoglobulin molecule (Ig molecule). The Ig may be of any class or subclass (IgG1, IgG2, IgG3, IgG4, IgA, IgE, IgM etc). A polypeptide chain of an Ig molecule folds into a series of parallel beta strands linked by loops. In the variable region, three of the loops constitute the “complementarity determining regions” (CDRs) which determine the antigen binding specificity of the immunoglobulin molecule. An IgG molecule comprises at least two heavy (H) chains and two light (L) chains inter-connected by disulfide bonds, or an antigen binding fragment thereof. Each heavy chain is comprised of a heavy chain variable region (abbreviated herein as VH) and a heavy chain constant region. The heavy chain constant region is comprised of three domains, CHL CH2 and CH3. Each light chain is comprised of a light chain variable region (abbreviated herein as VL) and a light chain constant region. The light chain constant region is comprised of one domain, CL. The VH and VL regions can be further subdivided into regions of hypervariability, termed complementarity determining regions (CDRs) with are hypervariable in sequence and/or involved in antigen recognition and/or usually form structurally defined loops, interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, FR4. In some embodiments of this disclosure, at least some or all of the amino acid sequences of FR1, FR2, FR3, and FR4 are part of the “non-CDR loop” of the dual binding moieties and the first and second binding moieties described herein. As shown in FIG. 1, a variable domain of an immunoglobulin molecule has several beta strands that are arranged in two sheets. The variable domains of both light and heavy immunoglobulin chains contain three hypervariable loops, or complementarity-determining regions (CDRs). The three CDRs of a V domain (CDR1, CDR2, CDR3) cluster at one end of the beta barrel. The CDRs are the loops that connect beta strands BC, C′C″, and FG of the immunoglobulin fold, whereas the bottom loops that connect beta strands A-B, CC′, C″ -D and EF of the immunoglobulin fold, and the top loop that connects the D-E strands of the immunoglobulin fold are the non-CDR loops. In some embodiments of this disclosure, at least some amino acid residues of a constant domain, CH1, CH2, or CH3, are part of the “non-CDR loop” of the dual binding moieties described herein. Non-CDR loops comprise, in some embodiments, one or more of AB, CD, EF, and DE loops of a C1-set domain of an Ig or an Ig-like molecule; AB, CC′, EF, FG, BC, and EC′ loops of a C2-set domain of an Ig or an Ig-like molecule; DE, BD, GF, A(A1A2)B, and EF loops of I(Intermediate)-set domain of an Ig or Ig-like molecule.

Within the variable domain, the CDRs are believed to be responsible for antigen recognition and binding, while the FR residues are considered a scaffold for the CDRs. However, in certain cases, some of the FR residues play an important role in antigen recognition and binding. Framework region residues that affect Ag binding are divided into two categories. The first are FR residues that contact the antigen, thus are part of the binding-site, and some of these residues are close in sequence to the CDRs. Other residues are those that are far from the CDRs in sequence, but are in close proximity to it in the 3-D structure of the molecule, e.g., a loop in heavy chain.

In some embodiments, the non-CDR loop within a dual binding moiety is modified to generate an antigen binding site specific for a target antigen, such as a tumor antigen (e.g., EGFR, PSMA) or an immune checkpoint protein (e.g., CTLA4, PD-1). It is contemplated that various techniques can be used for modifying the non-CDR loop, e.g., site-directed mutagenesis, random mutagenesis, insertion of at least one amino acid that is foreign to the non-CDR loop amino acid sequence, amino acid substitution. An antigen peptide is inserted into a non-CDR loop, in some examples. In some examples, an antigenic peptide is substituted for the non-CDR loop. The modification, to generate an antigen binding site, is in some cases in only one non-CDR loop. In other instances, more than one non-CDR loop are modified. For instance, in some cases, the modification is in any one of the non-CDR loops shown in FIG. 1, i.e., A-B, C-C′, C″-D, E-F, and D-E. In some cases, the modification is in the D-E loop. In other cases the modifications are in all four of A-B, C-C′, C″-D, E-F loops.

In certain examples, a dual binding moiety of a pro immune modulating molecule is bound to the target binding domain via its non-CDR loops, such as A-B, C-C′, C″-D, or E-F loop and is bound to a bulk serum protein or to a target, such as an immune modulatory protein, via its CDRs, such as B-C, C′-C″, and F-G loop. In certain examples, a dual binding moiety of a pro immune modulating molecule is bound to a dual binding moiety target instead of a bulk serum protein, such as an immune modulatory protein, via its non-CDR loops, such as A-B, C-C′, C″-D, or E-F loop and is bound to a target binding domain via its CDRs, such as B-C, C′-C″, or F-G loop. In certain examples, a dual binding moiety of a pro immune modulating molecule is bound to an immune modulatory protein, such as PD-L1, via its non-CDR loops, such as A-B, CC′, C″D, and EF loop and is bound a target binding domain via its CDRs, such as B-C, C′C″, and FG loop. In certain examples, a dual binding moiety of a pro immune modulating molecule is bound to an immune modulatory protein, such as PD-L1, via its CDRs, such as BC, C′C″, and F-G loop, and to a target binding domain by its non-CDR loops, such as one or more of as A-B, CC′, C″ D, or EF loop. In certain examples, a dual binding moiety of a pro immune modulating molecule is bound to a tumor antigen, such as PSMA, via its CDRs, such as BC, C′C″, and F-G loop, and to a target binding domain by its non-CDR loops, such as one or more of as A-B, CC′, C″ D, or EF loop. In certain examples, a dual binding moiety of a pro immune modulating molecule is bound to albumin via its CDRs, such as BC, C′C″, and F-G loop, and to a target binding domain (such as a CD40 antibody or a CTLA4 antibody) by its non-CDR loops, such as one or more of as A-B, CC′, C″ D, or EF loop. In certain examples, a dual binding moiety of a pro immune modulating molecule is bound to a tumor antigen, such as PSMA, via its CDRs, such as BC, C′C″, and F-G loop, and to a target binding domain (such as a CD40 antibody or a CTLA4 antibody) by its non-CDR loops, such as one or more of as A-B, CC′, C″ D, or EF loop. In some cases, a dual binding moiety of a pro immune modulating molecule is bound to a target binding domain via a non-CDR loop from a constant domain, a C1-set domain, a C2-set domain, an I-domain, as described above, and to a target or the bulk serum protein via its CDRs.

In certain examples of a conditionally active multivalent target-binding protein of this disclosure, the first binding moiety is bound to the first target antigen binding domain via its non-CDR loops, such as A-B, C-C′, C″-D, or E-F loop and is bound to a first target, such as an immune modulatory protein, via its CDRs, such as B-C, C′-C″, and F-G loop. In certain examples, the first binding moiety is bound to a first target, such as an immune modulatory protein, via its non-CDR loops, such as A-B, C-C′, C″-D, or E-F loop and is bound to the first target antigen binding domain via its CDRs, such as B-C, C′-C″, or F-G loop. In certain examples, the first binding moiety is bound to an immune modulatory protein, such as PD-L1, via its non-CDR loops, such as A-B, CC′, C″D, and EF loop and is bound to the first target antigen binding domain via its CDRs, such as B-C, C′C″, and FG loop. In certain examples, the first binding moiety is bound to an immune modulatory protein, such as PD-L1, via its CDRs, such as BC, C′C″, and F-G loop, and to the first target antigen binding domain by its non-CDR loops, such as one or more of as A-B, CC′, C″ D, or EF loop. In some cases, the first binding moiety is bound to the first target antigen binding domain via a non-CDR loop from a constant domain, a C1-set domain, a C2-set domain, an I-domain, as described above, and to the first target via its CDRs.

In certain examples, the second binding moiety is bound to the second target antigen binding domain via its non-CDR loops, such as AB, C-C′, C″ D, or EF loop and is bound to a second target, such as an immune modulatory protein, via its CDRs, such as BC, C′C″, and F-G loop. In certain examples, the second binding moiety is bound to a second target, such as an immune modulatory protein, via its non-CDR loops, such as AB, CC′, C″ D, or EF loop and is bound to the second target antigen binding domain via its CDRs, such as BC, C′C″, or FG loop. In certain examples, the second binding moiety is bound to an immune modulatory protein, such as PD-L1, via its non-CDR loops, such as AB, CC′, C″ D, and EF loop and is bound to the second target antigen binding domain via its CDRs, such as BC, C′C″, and FG loop. In certain examples, the second binding moiety is bound to an immune modulatory protein, such as PD-L1, via its CDRs, such as BC, C′C″, and FG loop, and to the second target antigen binding domain by its non-CDR loops, such as one or more of as AB, CC′, C″ D, or EF loop. In some cases, the second binding moiety is bound to the second target antigen binding domain via a non-CDR loop from a constant domain, a C1-set domain, a C2-set domain, an I-domain, as described above, and to the second target via its CDRs.

The dual binding moieties of a pro immune modulating molecule, the first and the second binding moieties of a conditionally active multivalent target-binding protein, in certain embodiments, are any kind of polypeptide, such as a natural peptide, a synthetic peptide, or a fibronectin scaffold. An engineered scaffold comprises, for example, sdAb, a scFv, a Fab, a VHH, a fibronectin type III domain, immunoglobulin-like scaffold (as described in Halaby et al., 1999. Prot Eng 12(7):563-571), DARPin, cystine knot peptide, lipocalin, three-helix bundle scaffold, protein G-related albumin-binding module, or a DNA or RNA aptamer scaffold.

In some embodiments, the first and the second target antigen binding domains of a conditionally active multivalent target-binding protein are specific for target antigens expressed on the surface of a diseased cell or tissue, for example a tumor or a cancer cell. In some embodiments, the first and the second target antigen binding domains of a conditionally active multivalent target-binding protein are independently specific for target antigens selected from EpCAM, EGFR, HER-2, HER-3, c-Met, FoIR, PSMA, CD38, BCMA, CEA, 5T4, AFP, B7-H3, Cadherin-6, CAIX, CD117, CD123, CD138, CD166, CD19, CD20, CD205, CD22, CD30, CD33, CD352, CD37, CD44, CD52, CD56, CD70, CD71, CD74, CD79b, DLL3, EphA2, FAP, FGFR2, FGFR3, GPC3, gpA33, FLT-3, gpNMB, HPV-16 E6, HPV-16 E7, ITGA2, ITGA3, SLC39A6, MAGE, mesothelin, Muc1, Muc16, NaPi2b, Nectin-4, P-cadherin, NY-ESO-1, PRLR, PSCA, PTK7, ROR1, SLC44A4, SLTRK5, SLTRK6, STEAP1, TIM1, Trop2, and WT1. In some embodiments, the first and the second target antigen binding domains of a conditionally active multivalent target-binding protein are independently specific for target antigens selected from CTLA-4, CD27, CD137, 2B4, TIGIT, CD155, ICOS, HVEM, CD40L, LIGHT, TIM-1, OX40, DNAM-1, PD-L1, PD1, PD-L2, CD8, CD40, CEACAM1, CD48, CD70, A2AR, CD39, CD73, B7-H3, B7-H4, BTLA, IDO1, IDO2, TDO, KIR, LAG-3, TIM-3, VISTA, IL6-R, IL-6, TNFα, CD19, CD20, CD22, CD52, integrin a4, integrin a4b7, CD11a, CTLA4-Ig fusion, IL-17, IL12/23, IL12, IL23, and TGF-beta.

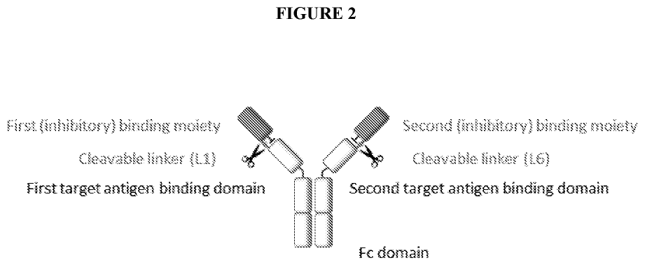

In some cases, the first and the second binding moieties are specific for an immune modulator protein, e.g., PD-L1, and the first and the second target antigen binding domain are specific for CTLA4, as shown in FIG. 2. In various embodiments, the conditionally active multivalent target-binding protein achieves a global inhibition of PD-L1 when in the global configuration state as well as after activation by cleavage of the cleavable linker. The PD-L1 inhibition is achieved both outside and inside tumor. Without being bound by any particular theory, it is contemplated that this global inhibition is tolerated because PD-L1 inhibition has a high therapeutic window, PD-L1 inhibition does not require Fc-effector function. In some cases, the global inhibition of PD-L1 is beneficial. Upon activation, the multivalent protein is able to locally inhibit CTLA4, inside the tumor. This, in some cases, expands the narrow therapeutic window for existing anti-CTLA4 therapies, and the conditionally active multivalent target-binding protein maintains the Fc-fragment for effector function, such as intratumoral Treg depletion.

In some embodiments, the binding moieties comprises a binding site for a bulk serum protein. In some embodiments, the CDRs provide a binding site for the bulk serum protein. The bulk serum protein is, for example, a globulin, a serum albumin protein (e.g., HSA/ALB), transferrin, IgG1, IgG2, IgG4, IgG3, IgA monomer, Factor XIII, Fibrinogen, IgE, or pentameric IgM. In some embodiments, the binding moiety comprises a binding site for an immunoglobulin light chain. In some embodiments, the CDRs provide a binding site for the immunoglobulin light chain. The immunoglobulin light chain is, for example, an Igκ free light chain or an Igλ free light chain.

Examples of first and second target antigen binding domains of conditionally active multivalent target-binding proteins include, but are not limited to, a variable heavy domain (VH), a variable light domain (VL), a scFv comprising a VH and a VL domain, a single domain antibody (sdAb), or a variable domain of camelid derived nanobody (VHH), a non-Ig binding domain, e.g., antibody mimetic, such as anticalins, affilins, affibody molecules, affimers, affitins, alphabodies, avimers, DARPins, fynomers, kunitz domain peptides, and monobodies, a ligand or peptide. In some embodiments, the first or the second target antigen binding domain is a VHH domain. In some embodiments, first or second target antigen binding domain is a sdAb. In some instances, the first target antigen binding domain is specific for a tumor antigen, such as EGFR or PSMA,and the binding of the first target antigen binding domain to its target, i.e., EGFR or PSMA is inhibited by masking of the binding moiety, via its non-CDR loops.

In one aspect, cleavable linkers of a dual binding moieties of pro immune modulating molecules or that of the first and second binding moieties of conditionally activated multivalent target-binding proteins comprise a polypeptides having a sequence recognized and cleaved in a sequence-specific manner. The cleavage, in certain examples, is enzymatic, based on pH sensitivity of the cleavable linker, or by chemical degradation. In some examples, protease cleavable linkers comprise cleavage sitesrecognized in a sequence-specific manner by a matrix metalloprotease (MMP), for example a MMP9. In some cases, a protease cleavable linker recognized by MMP9 comprises a polypeptide having an amino acid sequence PR(S/T)(L/I)(S/T). In some cases, a protease cleavable linker recognized by a MMP9 comprises a polypeptide having an amino acid sequence LEATA . In some cases, a protease cleavable linker is recognized in a sequence-specific manner by a MMP11. In some cases, a protease cleavable linker recognized by a MMP11 comprises a polypeptide having an amino acid sequence GGAANLVRGG (SEQ IN NO: 3). In some cases, a protease cleavable linker is recognized by a protease disclosed in Table 1. In some cases, a protease cleavable linker is recognized by a protease disclosed in Table 1 comprises a polypeptide having an amino acid sequence selected from a sequence disclosed in Table 1 (SEQ ID NOS: 1-42),In some cases, a cleavable linker comprise a sequence as set forth in SEQ ID No. 100 or SEQ ID No. 101.

Proteases are proteins that cleave proteins, in some cases, in a sequence-specific manner. Proteases include but are not limited to serine proteases, cysteine proteases, aspartate proteases, threonine proteases, glutamic acid proteases, metalloproteases, asparagine peptide lyases, serum proteases, cathepsins, Cathepsin B, Cathepsin C, Cathepsin D, Cathepsin E, Cathepsin K, Cathepsin L, kallikreins, hK1, hK10, hK15, plasmin, collagenase, Type IV collagenase, stromelysin, Factor Xa, chymotrypsin-like protease, trypsin-like protease, elastase-like protease, subtilisin-like protease, actinidain, bromelain, calpain, caspases, caspase-3, Mir1-CP, papain, HIV-1 protease, HSV protease, CMV protease, chymosin, renin, pepsin, matriptase, legumain, plasmepsin, nepenthesin, metalloexopeptidases, metalloendopeptidases, matrix metalloproteases (MMP), MMP1, MMP2, MMP3, MMP8, MMP9, MMP13, MMP11, MMP14, urokinase plasminogen activator (uPA), enterokinase, prostate-specific antigen (PSA, hK3), interleukin-1β converting enzyme, thrombin, FAP (FAP-α), dipeptidyl peptidase, dipeptidyl peptidase IV (DPPIV/CD26), type II transmembrane serine proteases (TTSP), neutrophil serine protease, cathepsin G, proteinase 3, neutrophil serine protease 4, mast cell chymase, and mast cell tryptases.

TABLE 1
Exemplary Proteases and Protease Recognition Sequences

| | Cleavage | SEQ |
|---|---|---|
| Protease | Domain Sequence | ID NO: |
| MMP7 | KRALGLPG | 1 |
| MMP7 | (DE)₈RPLALWRS(DR)₈ | 2 |
| MMP9 | PR(S/T)(L/I)(S/T) | 3 |
| MMP9 | LEATA | 4 |
| MMP11 | GGAANLVRGG | 5 |
| MMP14 | SGRIGFLRTA | 6 |
| MMP | PLGLAG | 7 |
| MMP | PLGLAX | 8 |
| MMP | PLGC(me)AG | 9 |
| MMP | ESPAYYTA | 10 |
| MMP | RLQLKL | 11 |
| MMP | RLQLKAC | 12 |
| MMP2, MMP9, MMP14 | EP(Cit)G(Hof)YL | 13 |
| Urokinase plasminogen activator | SGRSA | 14 |
| (uPA) | | |
| Urokinase plasminogen activator | DAFK | 15 |
| (uPA) | | |
| Urokinase plasminogen activator | GGGRR | 16 |
| (uPA) | | |
| Lysosomal Enzyme | GFLG | 17 |
| Lysosomal Enzyme | ALAL | 18 |
| Lysosomal Enzyme | FK | 19 |
| Cathepsin B | NLL | 20 |
| Cathepsin D | PIC(Et)FF | 21 |
| Cathepsin K | GGPRGLPG | 22 |
| Prostate Specific Antigen | HSSKLQ | 23 |
| Prostate Specific Antigen | HSSKLQL | 24 |
| Prostate Specific Antigen | HSSKLQEDA | 25 |
| Herpes Simplex Virus Protease | LVLASSSFGY | 26 |
| HIV Protease | GVSQNYPIVG | 27 |
| CMV Protease | GVVQASCRLA | 28 |
| Thrombin | F(Pip)RS | 29 |
| Thrombin | DPRSFL | 30 |
| Thrombin | PPRSFL | 31 |
| Caspase-3 | DEVD | 32 |
| Caspase-3 | DEVDP | 33 |
| Caspase-3 | KGSGDVEG | 34 |
| Interleukin 1β converting enzyme | GWEHDG | 35 |
| Enterokinase | EDDDDKA | 36 |
| FAP | KQEQNPGST | 37 |
| Kallikrein 2 | GKAFRR | 38 |
| Plasmin | DAFK | 39 |
| Plasmin | DVLK | 40 |
| Plasmin | DAFK | 41 |
| TOP | ALLLALL | 42 |

Proteases are known to be secreted by some diseased cells and tissues, for example tumor or cancer cells, creating a microenvironment that is rich in proteases or a protease-rich microenvironment. In some case, the blood of a subject is rich in proteases. In some cases, cells surrounding the tumor secrete proteases into the tumor microenvironment. Cells surrounding the tumor secreting proteases include but are not limited to the tumor stromal cells, myofibroblasts, blood cells, mast cells, B cells, NK cells, regulatory T cells, macrophages, cytotoxic T lymphocytes, dendritic cells, mesenchymal stem cells, polymorphonuclear cells, and other cells. In some cases, proteases are present in the blood of a subject, for example proteases that target amino acid sequences found in microbial peptides. This feature allows for targeted therapeutics such as antigen binding proteins to have additional specificity because T cells will not be bound by the antigen binding protein except in the protease rich microenvironment of the targeted cells or tissue. A dual binding moiety comprising the cleavable linker thus masks the binding of a target binding domain; and the first and second binding moieties comprising a cleavable linker mask binding of a first or a second target antigen binding domain to their respective targets.

### Fc Region of the Target-Binding Proteins

In some embodiments, the conditionally active multivalent target-binding protein (such as a pro immune modulating molecule) comprises an Fc domain comprising sdAb-Fc, scFv-Fc, or an antibody. In one embodiment, the conditionally active multivalent target-binding protein comprises an Fc domain comprising a first monomeric Fc domain (Fc1) and a second monomeric Fc domain (Fc2). In some embodiments, Fc1 comprises CH3-Fc1 and CH2-Fc1; and Fc2 comprising CH3-Fc2 and CH2-Fc2, wherein CH3-Fc1 and CH3-Fc2 associate to form a heterodimer. In some embodiments, the Fc domain comprises a homodimer comprises CH3 and CH2 domains. The Fc domain, in certain examples, stabilizes the multivalent target-binding protein in circulation.

An Fc domain conjugated to an effector molecule is referred to as an Fc domain molecule or an Fc domain fusion protein. The fusion protein can include, for example, a VH, an engineered antibody domain, a diabody, a scFv, a cytokine, a toxin, an enzyme, and/or a ligand attached an Fc domain, wherein the Fc domain comprises a CH3 and a CH2 domain. CH2 and CH3 domain molecules are small in size, usually less than 15 kD. It is contemplated that in certain embodiments the CH2 and CH3 domain molecules are conjugated to an effector molecule or a label. In other embodiments, CDRs/hypervariable amino acid sequences are inserted into the CH3 and/or CH2 domain. In such embodiments, the CH2 or CH3 domain vary in size depending on the length of CDR/hypervariable amino acid sequence inserted in the loops regions, how many CDRs are inserted and whether another molecule (such as an effector molecule or label) is conjugated to the CH2 or CH3 domain. In some embodiments, the Fc domain does not comprise additional constant domains (such as CH1). In one embodiment, the CH3 and/or CH2 domain is from IgG, IgA or IgD. In another embodiment, the CH3 and/or CH2 domain is from IgE or IgM. CH2 and CH3 domain molecules can be glycosylated or unglycosylated. For example, a recombinant CH2 or CH3 domain can be expressed in an appropriate mammalian cell to allow glycosylation of the molecule.

In some embodiments, the conditionally active multivalent target-binding protein disclosed herein is an Fc domain molecule or an Fc fusion protein. In some embodiments, the conditionally active multivalent target-binding target-binding protein comprises a Fc region comprising a first and a second monomeric Fc domain, Fc1 and Fc2, respectively, which monomeric Fc domains associate with each other to form a heterodimeric Fc region. The N-termini of the Fc1 and the Fc2 of the Fc domain fusion proteins are attached to first and the second target antigen binding domains, respectively.

In one embodiment, the conditionally active multivalent target-binding protein (such as a pro immune modulating molecule) is a Fc domain fusion protein. In some embodiments, a VHH domain capable of binding to a first target is attached to the N-terminus of Fc1, and a scFv capable of binding to a second target is attached to the N-terminus of Fc2, and wherein the VHH domain is further attached to a binding moiety via the non-CDR loops of the binding moiety, and a cleavable linker, which binding moiety obstructs the binding of the VHH domain to the first target.

In some embodiments, the pro immune modulating molecule comprises an antibody with an Fc region as the target antibody domain and the Fc region comprises mutations in at least one of the CH2 and CH3 domains, e.g., mutation in at least one or both of positions H310 and H435 to H310A and H435A (numbering corresponding to EU numbering for IgG1).

The conditionally active multivalent target-binding proteins described herein have a binding inactive and a binding active configuration. The binding inactive configuration is such that the first and the second binding moieties obstruct the first and the second target antigen binding domains, and render the protein incapable of binding to its first and second targets. In some embodiments, the conditionally active multivalent target-binding proteins do not have target-domain binding capability until all the protease cleavage sites in the cleavable linkers connected to the first and the second binding moieties are cleaved and the first and the second target antigen binding domains are no longer obstructed by a binding moiety comprising non-CDR loops.

In some embodiments, various domains of the heteromultimeric conditionally active multivalent target-binding proteins described herein are connected to each other by internal linkers. In embodiments where conditionally active multivalent target-binding protein comprises a first binding moiety, a first target antigen binding domain, a first Fc domain (Fc1) comprising CH3-Fc1 and CH2-Fc1; a second binding moiety, a second target antigen binding domain, a second Fc domain (Fc2) comprising CH3-Fc2 and CH2-Fc2, the linkers are as follows: L1 links first binding moiety and the first target antigen binding domain, L2 links first antigen binding domain and the N-terminus of CH2-Fc1; L3 links CH2-Fc1 and CH3-Fcl, L4 links CH3-Fc2 and CH2-Fc2, L5 links the N-terminus of CH2-Fc2 and second target antigen binding domain, L6 links second target antigen binding domain and the second binding moiety. At least, linkers L1 and L6 are cleavable linkers.

In some embodiments where pro immune modulating molecule comprises a target binding domain comprising two light chain and two heavy chain polypeptides, and two dual binding moieties, the various domains are connected by linkers are as follows: L1 links one of the dual binding moieties to one of the heavy chain polypeptides or one of the light chain polypeptides , L2 links the other dual binding moiety to the other heavy chain polypeptide or the other light chain polypeptide; L3 links CH2 and CH3 domains of one of the heavy chain polypeptides, L4 links CH2 and CH3 domains of the other heavy chain polypeptides, L5 links the CH2 domain of one of the heavy chain polypeptides with the variable region and L6 links the CH2 domain of the other heavy chain polypeptides with the variable region. In some instances, at least, linkers L1 and L2 are cleavable linkers, for examples, having a sequence as set forth in SEQ ID No. 100 or SEQ ID No. 101.

Linkers L, L2, L3, L4, L5, and L6 have an optimized length and/or amino acid composition. In some embodiments, linkers L, L2, L3, L4, L5, and L6 are 3-200 amino acids in length. In some embodiments, linkers L, L2, L3, L4, L5, and L6 have the same length or amino acid composition. In other embodiments, linkers L, L2, L3, L4, L5, and L6 have different amino acid compositions. In other embodiments, linkers L, L2, L3, L4, L5, and L6 have different lengths. In certain embodiments, internal linkers L, L2, L3, L4, L5, and L6 are “short,” such as, consist of 0, 1, 2, 3, 4, 5, 6, 7, 8, 9, 10, 11 or 12 amino acid residues. Thus, in certain instances, the linkers consist of about 12 or less amino acid residues. In the case of 0 amino acid residues, the linker is a peptide bond. In certain embodiments, linkers L1, L2, L3, L4, L5, and L6 consist of 15, 20 or 25 amino acid residues. In some embodiments, the linkers consist of about 3 to about 15, for example 8, 9 or 10 contiguous amino acid residues. Regarding the amino acid composition of the linkers L1, L2, L3, L4, L5, and L6, peptides are selected with properties that confer flexibility to the antigen-binding proteins, do not interfere with the target-binding domain as well as resist cleavage from proteases, unless the protease cleavage sites are located within the linkers. Examples of internal linkers suitable for linking the domains in the antigen-binding proteins include but are not limited to (GS)ₙ, (GGS)ₙ, (GGGS)ₙ, (GGSG)ₙ, (GGSGG)ₙ, or (GGGGS)ₙ, wherein n is 1, 2, 3, 4, 5, 6, 7, 8, 9, or 10. In one embodiment, internal linker L1, L2, L3, L4, L5, and L6 is (GGGGS)₄ , (GGGGSGGGSGGGGSGGGS) or (GGGGS)₃. In some cases, linkers L1 and L6 are cleavable linkers and comprise a sequence selected from SEQ ID No. 100 and SEQ ID No. 101.

In certain instances, the linkers L1 and L6 comprise protease cleavage sites. Such protease sensitive linkers are, in certain embodiments, sensitive to protease present in specific tissue or intracellular compartments (e.g., MMPs, furin, cathepsin B). Example sequences for such protease sensitive cleavable linkers include but are not limited to (PLGLWA)ₙ(SEQ ID NO: 43), (RVLAEA)ₙ(SEQ ID NO: 44); (EDVVCCSMSY)n (SEQ ID NO: 45), (GGIEGRGS)ₙ (SEQ ID NO: 46), which are recognized by MMP-1, and (GFLG)n (SEQ ID NO: 47) which are recognized by furin. The linkers containing protease cleavage sites play a role in activation of the conditionally active multivalent target-domain binding protein.

It is contemplated that in some embodiments the activatable binding proteins of this disclosure (such as a pro immune modulating molecule or a conditionally active multivalent target-binding protein) is no more than 100 kD, no more than 75 kD, no more than 50 kD, no more than 25 kD, no more than 20 kD, no more than 15 kD, or no more than 10 kD, upon its activation by protease cleavage. Prior to cleavage and activation, the conditionally active protein is, in certain embodiments, no more than 100 kD, no more than 75 kD, no more than 50 kD, no more than 25 kD, no more than 20 kD, or no more than 15 kD.

### Fc-Domain Heterodimerization

Conventional IgG antibodies are multivalent and monospecific, the assembly of which depends upon in vivo homodimerization of two identical heavy chains (HCs), which is mediated by homodimeric associations between CH3 domains, and subsequently disulfide linkages between each HC and each light chain (LC), in B cells. Thus, the development of bsAbs, using intact IgG formats with wild-type HCs and LCs, faces HC-HC and HC_{VH-CH1}LC mispairing problems. Accordingly, in some embodiments, the heterodimeric Fc region of the conditionally active multivalent target-bindingprotein disclosed herein is advantageous in terms of avoiding HC mispairing problem.

Although the wild-type Fc region is a homodimer of polypeptides, the Fc domains disclosed herein, in some examples, comprise amino acid substitutions such that they do not form homodimers. The monomeric Fc domains, Fc1 and Fc2, are in some embodiments IgG Fc. In some embodiments,the monomeric Fc domains, Fc1 and Fc2, are from other immunoglobulin subclasses including IgA, IgE, IgD, and IgM.

The heterodimer Fc region of an activatable binding protein of this disclosure, such as a pro immune modulating molecule or a conditionally active multivalent target-binding protein described herein comprises a variant CH3 constant domain comprising amino acid mutations that promote the formation of said heterodimer with stability comparable to a native homodimeric Fc, and a CH2 constant domain. The wild-type Fc is homodimeric in nature and this feature is driven by both hydrophobic interactions at the center of the CH3 interface and symmetric electrostatic interactions around the rim of the hydrophobic core. In some embodiments, an Fc domain described herein comprises amino acid substitutions such that they do not form homodimers. In some embodiments, an Fc domain described herein comprises amino acid substitutions that favor formation of heterodimers over homodimers. In some embodiments, a variant Fc domain is created using (i) symmetric-to-asymmetric steric complementarity design (e.g., KiH, HA-TF, and ZW1), (ii) charge-to-charge swap (e.g., DD-KK), (iii) charge-to-steric complementarity swap plus additional long-range electrostatic interactions (e.g., EW-RVT), and (iv) isotype strand swap [e.g., strand-exchange engineered domain (SEED)]. Strand exchange mutations include, for example, IgA-derived 45 residues on IgG1 CH3-Fc1 and IgG1-derived 57 residues on IgA CH3-Fc2, or vice versa. Examples of symmetric-to-asymmetric sterically complementary mutations include HA-TF (S364H/F405A in Fc1-CH3 or CH3A and Y349T/T394F in Fc2-CH3 or CH3B), ZW1(T350V/L351Y/F405A/Y407V in Fc1-CH3 or CH3A and T350V/T366L/K392L/T394W in Fc2-CH3 or CH3B). In some embodiments, an Fc variant is generated using the “Knobs-into-holes (KiH)” approach where Fc1 comprises a T366W “knob” mutation, in Fc1-CH3 or CH3A, and Fc2 comprises T366S/L368A/Y407V “hole” mutations in Fc2-CH3 or CH3B domain. In some embodiments, anFc variant is generated using the “Knobs-into-holes (KiH)” plus disulfide bond approach, KiH_{S—S}, where Fc1 comprises a T366W/S354C “knob” mutation, in Fc1-CH3 or CH3A, and Fc2 comprises T366S/L368A/Y407V/Y349C “hole” mutations in Fc2-CH3 or CH3B domain. In such exemplary embodiments, the heterodimerization is favored through hydrophobic interactions at the core of the Fc1-CH3 or CH3A and Fc2-CH3 or CH3B interface. Examples of charge-charge swap mutations, where the Fc heterodimer favoring interaction is based on electrostatic complementarity include DD-KK (K409D/K392D in Fc1-CH3 or CH3A and D399K/E356K in Fc2-CH3 or CH3B, or vice versa). Examples of charge-to-steric complementarity swap plus additional long-range electrostatic interaction mutations include EW-RVT (K360E/K409W in Fc1-CH3 or CH3A and Q347R/D399V/F405T in Fc2-CH3 or CH3B, or vice versa); EW-RVT_{S—S} (K360E/K409W/Y349C in Fc1-CH3 or CH3A and Q347R/D399V/F405T/S354C in Fc2-CH3 or CH3B, or vice versa), which comprises an inter-CH3 S—S bond. In some embodiments, the Fc variant is generated using hydrophobic or steric complementarity plus electrostatic complementarity, such as 7.8.60 (K360D/D399M/Y407A in Fc1-CH3 or CH3A and E345R/Q347R/T366V/K409V in Fc2-CH3 or CH3B, or vice versa).

In certain embodiments, the heterodimer forming Fc variants described herein are generated through directed evolution combined with yeast surface display and high-throughput screening. For example, in some embodiments, a combinatorial heterodimeric Fc library display system is developed by mating two haploid yeast cell lines; one haploid cell line displaying an Fc chain library (CH3-Fc1 or CH3A) with mutations in one CH3 domain on the yeast cell surface, and the other cell line secreting an Fc chain library (CH3-Fc2 or CH3B) with mutations in the other CH3 domain. In the mated cells, secreted CH3-Fc2 or CH3B is displayed on the cell surface through heterodimerization with the displayed CH3-Fc1 or CH3A. Fluorescence-based detection of this interaction enables screening of the library for heterodimeric Fc variants by flow cytometry.

An antibody that includes a wild-type Fc domain has the ability to interact with neonatal Fc-receptor (FcRn) in a pH dependent manner; this interaction confers extended serum half-life. The residues important for the high-affinity interaction of Fc domain and FcγR are located within the CH2 domain. Accordingly, in some embodiments, the Fc heterodimer of an activatable binding protein, such as a pro immune modulating molecule or a conditionally active multivalent target-binding protein comprises CH2 domains which have wild type IgG sequence.

### Activated Binding Proteins

The conditionally active multivalent target-binding proteins described herein are activated by cleavage of cleavable linkers connecting the first binding moiety to the first target antigen binding domain and the second binding moiety to the second target antigen binding domain. It is contemplated that the activated conditionally active multivalent target-binding protein binds to a pair of target antigens involved in and/or associated with a disease, disorder or condition. In particular, target antigens associated with a proliferative disease, a tumorous disease, an inflammatory disease, an immunological disorder, an autoimmune disease, an infectious disease, a viral disease, an allergic reaction, a parasitic reaction, a graft-versus-host disease or a host-versus-graft disease are contemplated to be the target for the activated multivalent proteins disclosed herein. In some embodiments, one of the target antigens is a tumor antigen expressed on a tumor cell. In some embodiments, a target antigen is a on a tumor cell, virally infected cell, bacterially infected cell, damaged red blood cell, arterial plaque cell, or fibrotic tissue cell.

In some embodiments, a target antigen is a cell surface molecule such as a protein, lipid or polysaccharide. In some embodiments, one of the target antigens is an immune modulatory protein.

Target antigens, in some cases, are expressed on the surface of a diseased cell or tissue, for example a tumor or a cancer cell. Examples of target antigens include but are not limited to EpCAM, EGFR, HER-2, HER-3, c-Met, FoIR, PSMA, CD38, BCMA, CEA, 5T4, AFP, B7-H3, Cadherin-6, CAIX, CD117, CD123, CD138, CD166, CD19, CD20, CD205, CD22, CD30, CD33, CD352, CD37, CD44, CD52, CD56, CD70, CD71, CD74, CD79b, DLL3, EphA2, FAP, FGFR2, FGFR3, GPC3, gpA33, FLT-3, gpNMB, HPV-16 E6, HPV-16 E7, ITGA2, ITGA3, SLC39A6, MAGE, mesothelin, Muc1, Muc16, NaPi2b, Nectin-4, P-cadherin, NY-ESO-1, PRLR, PSCA, PTK7, ROR1, SLC44A4, SLTRK5, SLTRK6, STEAP1, TIM1, Trop2, and WT1.

In some embodiments, a target antigen for the first polypeptide of the conditionally active multivalent target-binding protein comprises a tumor antigen and the target antigen for the second polypeptide comprises an immune modulatory protein. In some embodiments, a target antigen for the first polypeptide of the conditionally active multivalent target-binding protein comprises a first immune modulatory protein and a target antigen for the second polypeptide comprises a second immune modulatory protein. Examples of immune modulatory proteins include but are not limited to CTLA-4, CD27, CD137, 2B4, TIGIT, CD155, ICOS, HVEM, CD40L, LIGHT, TIM-1, OX40, DNAM-1, PD-L1, PD1, PD-L2, CD8, CD40, CEACAM1, CD48, CD70, A2AR, CD39, CD73, B7-H3, B7-H4, BTLA, IDOL IDO2, TDO, KIR, LAG-3, TIM-3, VISTA, IL6-R, IL-6, TNFα, CD19, CD20, CD22, CD52, integrin a4, integrin a4b7, CD11a, CTLA4-Ig fusion, IL-17, IL12/23, IL12, IL23, and TGF-beta. Inhibitory immune checkpoint proteins to be inhibited in activating an immune response include but are not limited to A2AR, B7-H3, B7-H4, BTLA, CTLA-4, IDO, KIR, LAG3, PD-1, PD-L1, TIM-3, and VISTA. In some embodiments, binding of the conditionally active multivalent target-binding protein to an immune modulatory target protein is dependent upon protease cleavage of the inhibitory domain which restricts binding of the protein to the immune modulatory target protein only in the microenvironment of a diseased cell or tissue with elevated levels of proteases, for example in a tumor microenvironment.

In some embodiments, at least one of the first and the second target binding domains of the conditionally active multivalent target-binding proteins described herein specifically binds to A2AR. In some embodiments, at least one of the first and the second target binding domains of the conditionally active multivalent target-binding proteins described herein specifically binds to B7-H3. In some embodiments, at least one of the first and the second target antigen binding domain of the conditionally active multivalent target-binding proteins described herein specifically binds to B7-H4. In some embodiments, at least one of the first and the second target antigen binding domain of the conditionally active multivalent target-binding proteins described herein specifically binds to BTLA. In some embodiments, at least one of the first and the second target antigen binding domains of the conditionally active multivalent target-binding proteins described herein specifically binds to CTLA-4. In some embodiments, at least one of the first and the second target antigen binding domains of the conditionally active multivalent target-binding proteins described herein specifically binds to IDO. In some embodiments, at least one of the first and the second target antigen binding domains of the conditionally active multivalent target-binding proteins described herein specifically binds to KIR. In some embodiments, at least one of the first and the second target antigen binding domains of the conditionally active multivalent target-binding proteins described herein specifically binds to LAG3. In some embodiments, at least one of the first and the second target antigen binding domains of conditionally active multivalent target-binding proteins described herein specifically binds to PD-1. In some embodiments, at least one of the first and the second target antigen binding domains of the conditionally active multivalent target-binding proteins described herein specifically binds to PD-L1. In some embodiments, at least one of the first and the second target antigen binding domains of the conditionally active multivalent target-binding proteins described herein specifically binds to TIM-3. In some embodiments, at least one of the first and the second target antigen binding domains of the conditionally active multivalent target-binding proteins described herein specifically binds to VISTA.

The pro immune modulating molecules described herein are activated by cleavage of cleavable linkers connecting the dual binding moiety to the target antigen binding domain. It is contemplated that the activated immune modulating molecule binds to antibody targets, such as target antigens with a disease, disorder or condition. In particular, target antigens associated with a proliferative disease, a tumorous disease, an inflammatory disease, an immunological disorder, an autoimmune disease, an infectious disease, a viral disease, an allergic reaction, a parasitic reaction, a graft-versus-host disease or a host-versus-graft disease are contemplated to be the target for the activated multivalent proteins disclosed herein. In some embodiments, an antibody target is a tumor antigen expressed on a tumor cell. In some embodiments, an antibody target is a target antigen expressed on a virally infected cell, bacterially infected cell, damaged red blood cell, arterial plaque cell, or fibrotic tissue cell.

In some embodiments, an antibody target is a cell surface molecule such as a protein, lipid or polysaccharide. In some embodiments, an antibody target is an immune modulatory protein, such as an immune checkpoint protein.

Examples of antibody targets that are tumor antigens include but are not limited to EpCAM, EGFR, HER-2, HER-3, c-Met, FoIR, PSMA, CD38, BCMA, CEA, 5T4, AFP, B7-H3, Cadherin-6, CAIX, CD117, CD123, CD138, CD166, CD19, CD20, CD205, CD22, CD30, CD33, CD352, CD37, CD44, CD52, CD56, CD70, CD71, CD74, CD79b, DLL3, EphA2, FAP, FGFR2, FGFR3, GPC3, gpA33, FLT-3, gpNMB, HPV-16 E6, HPV-16 E7, ITGA2, ITGA3, SLC39A6, MAGE, mesothelin, Muc1, Muc16, NaPi2b, Nectin-4, P-cadherin, NY-ESO-1, PRLR, PSCA, PTK7, ROR1, SLC44A4, SLTRK5, SLTRK6, STEAP1, TIM1, Trop2, and WT1.

Examples of antibody targets that are immune modulatory proteins include but are not limited to CD27, CD137, 2B4, TIGIT, CD155, ICOS, HVEM, CD40L, LIGHT, TIM-1, OX40, DNAM-1, PD-L1, PD1, PD-L2, CTLA-4, CD8, CD40, CEACAM1, CD48, CD70, A2AR, CD39, CD73, B7-H3, B7-H4, BTLA, IDOL IDO2, TDO, KIR, LAG-3, TIM-3, or VISTA. Inhibitory immune checkpoint proteins to be inhibited in activating an immune response include but are not limited to A2AR, B7-H3, B7-H4, BTLA, CTLA-4, IDO, KIR, LAG3, PD-1, PD-L1, TIM-3, and VISTA.

In some embodiments, a target binding domain of an activated pro immune modulating molecule described herein specifically binds to A2AR, i.e., A2AR is an antibody target. In some embodiments, a target binding domain of ac activated pro immune modulating molecule described herein specifically binds to B7-H3, i.e., B7-H3 is an antibody target. In some embodiments, a target binding domain of ac activated pro immune modulating molecule described herein specifically binds to B7-H4, i.e., B7-H4 is an antibody target. In some embodiments, a target binding domain of ac activated pro immune modulating molecule described herein specifically binds to BTLA, i.e., BTLA is an antibody target. In some embodiments, a target binding domain of ac activated pro immune modulating molecule described herein specifically binds to CTLA-4, i.e., CTLA-4 is an antibody target. In some embodiments, a target binding domain of ac activated pro immune modulating molecule described herein specifically binds to IDO, i.e., IDO is an antibody target. In some embodiments, a target binding domain of ac activated pro immune modulating molecule described herein specifically binds to KIR, i.e., KIR is an antibody target. In some embodiments, a target binding domain of ac activated pro immune modulating molecule described herein specifically binds to LAG3, i.e., LAG3 is an antibody target. In some embodiments, a target binding domain of ac activated pro immune modulating molecule described herein specifically binds to PD-1, i.e., PD-1 is an antibody target. In some embodiments, a target binding domain of ac activated pro immune modulating molecule described herein specifically binds to PD-L1, i.e., PD-L1 is an antibody target. In some embodiments, a target binding domain of ac activated pro immune modulating molecule described herein specifically binds to TIM-3, i.e., TIM-3 is an antibody target. In some embodiments, a target binding domain of ac activated pro immune modulating molecule described herein specifically binds to VISTA, i.e., VISTA is an antibody target.

In some embodiments, an antibody target of a pro immune modulating molecule, a dual binding moiety target of a pro immune modulating molecule, a target antigen of the first and/or second target antigen binding domain of a conditionally active target-binding domain independently is a viral antigen. Examples of viral antigens include but are not limited to Hepatitis Viruses, Flaviviruses, Westnile Virus, Ebola Virus, Pox-Virus, Smallpox Virus, Measles Virus, Herpes Virus, Adenovirus, Papilloma Virus, Polyoma Virus, Parvovirus, Rhinovirus, Coxsackie virus, Polio Virus, Echovirus, Japanese Encephalitis virus, Dengue Virus, Tick Burne Encephalitis Virus, Yellow Fever Virus, Coronavirus, respiratory syncytial virus, parainfluenza virus, La Crosse Virus, Lassa Virus, Rabies Viruse, and Rotavirus antigens.

### Binding Protein Variants

As used herein, the term “binding protein variants” refers to variants and derivatives of the pro immune modulating molecules or the conditionally active multivalent target-binding proteins described herein. In certain embodiments, amino acid sequence variants are contemplated. For example, in certain embodiments amino acid sequence variants are contemplated to improve the binding affinity and/or other biological properties of the activatable proteins. Exemplary method for preparing amino acid variants include, but are not limited to, introducing appropriate modifications into the nucleotide sequence encoding the antibody, or by peptide synthesis. Such modifications include, for example, deletions from, and/or insertions into and/or substitutions of residues within the amino acid sequences of the antibody.

Any combination of deletion, insertion, and substitution can be made to the various domains to arrive at the final construct, provided that the final construct possesses the desired characteristics, e.g., antigen-binding. In certain embodiments, variants having one or more amino acid substitutions are provided. Sites of interest for substitution mutagenesis include the CDRs and framework regions. Amino acid substitutions may be introduced into the variable domains of the target-binding protein of interest and the products screened for a desired activity, e.g., retained/improved antigen binding, decreased immunogenicity, or improved antibody-dependent cell mediated cytotoxicity (ADCC) or complement dependent cytotoxicity (CDC). Both conservative and non-conservative amino acid substitutions are contemplated for preparing the variants.

In another example of a substitution to create a variant, one or more hypervariable region residues of a parent antibody are substituted. In general, variants are then selected based on improvements in desired properties compared to a parent antibody, for example, increased affinity, reduced affinity, reduced immunogenicity, increased pH dependence of binding. For example, an affinity matured variant antibody can be generated, e.g., using phage display-based affinity maturation techniques such as those described herein and known in the field.

Substitutions can be made in hypervariable regions (HVR) of a parent molecule to generate variants and variants are then selected based on binding affinity, i.e., by affinity maturation. In some embodiments of affinity maturation, diversity is introduced into the variable genes chosen for maturation by any of a variety of methods (e.g., error-prone PCR, chain shuffling, or oligonucleotide-directed mutagenesis). A secondary library is then created. The library is then screened to identify any antibody variants with the desired affinity. Another method to introduce diversity involves HVR-directed approaches, in which several HVR residues (e.g., 4-6 residues at a time) are randomized. HVR residues involved in antigen binding may be specifically identified, e.g., using alanine scanning mutagenesis or modeling. Substitutions can be in one, two, three, four, or more sites within a parent antibody sequence.

In some embodiments, a pro immune modulating molecule and a conditionally active multivalent target-binding protein as described herein comprises a VL and a VH domain with amino acid sequences corresponding to the amino acid sequence of a naturally occurring VL or VH domain, respectively, but that has been “humanized,” i.e., by replacing one or more amino acid residues in the amino acid sequence of said naturally occurring VL or VH domains (and in particular in the framework sequences) by one or more of the amino acid residues that occur at the corresponding position(s) in a VL or VH domain from a conventional 4-chain antibody from a human being (e.g., as indicated above). This can be performed in a manner known in the field, which will be clear to the skilled person, for example on the basis of the further description herein. Again, it should be noted that such humanized conditionally active multivalent target-binding antibodies of the disclosure are obtained in any suitable manner known per se and thus are not strictly limited to polypeptides that have been obtained using a polypeptide that comprises a naturally occurring VL and/or VH domain as a starting material. In some additional embodiments, pro immune modulating molecule and a conditionally active multivalent target-binding antibody, as described herein, comprises a VL and a VH domain with amino acid sequences corresponding to the amino acid sequence of a naturally occurring VL or VH domain, respectively, but that has been “camelized,” i.e., by replacing one or more amino acid residues in the amino acid sequence of a naturally occurring VL or VH domain from a conventional 4-chain antibody by one or more of the amino acid residues that occur at the corresponding position(s) in a VL or a VH domain of a heavy chain antibody. Such “camelizing” substitutions are preferably inserted at amino acid positions that form and/or are present at the VH-VL interface, and/or at the so-called Camelidae hallmark residues (see for example WO 94/04678 and Davies and Riechmann (1994 and 1996)). Preferably, the VH sequence that is used as a starting material or starting point for generating or designing the camelized single domain is preferably a VH sequence from a mammal, more preferably the VH sequence of a human being, such as a VH3 sequence. However, it should be noted that such camelized domains of the activatable binding proteins of the disclosure, in certain embodiments, are obtained in any suitable manner known in the field and thus are not strictly limited to polypeptides that have been obtained using a polypeptide that comprises a naturally occurring VL and/or VH domain as a starting material. For example, both “humanization” and “camelization” is performed by providing a nucleotide sequence that encodes a naturally occurring VL and/or VH domain, respectively, and then changing, one or more codons in said nucleotide sequence in such a way that the new nucleotide sequence encodes a “humanized” or “camelized” binding protein, respectively. This nucleic acid can then be expressed, so as to provide the desired target-antigen binding capability. Alternatively, in other embodiments, a “humanized” or “camelized” conditionally active antibody is synthesized de novo using known peptide synthesis technique from the amino acid sequence of a naturally occurring antibody comprising a VL and/or VH domain. In some embodiments, a “humanized” or “camelized” binding protein is synthesized de novo using known peptide synthesis technique from the amino acid sequence or nucleotide sequence of a naturally occurring antibody comprising a VL and/or VH domain, respectively, a nucleotide sequence encoding the desired humanized or camelized binding protein of the disclosure, respectively, is designed and then synthesized de novo using known techniques for nucleic acid synthesis, after which the nucleic acid thus obtained is expressed in using known expression techniques, so as to provide the desired binding protein of the disclosure.

Other suitable methods and techniques for obtaining the binding proteins of the disclosure and/or nucleic acids encoding the same, starting from naturally occurring sequences for VL or VH domains for example comprises combining one or more parts of one or more naturally occurring VL or VH sequences (such as one or more framework (FR) sequences and/or complementarity determining region (CDR) sequences), and/or one or more synthetic or semi-synthetic sequences, and/or a naturally occurring sequence for a CH2 domain, and a naturally occurring sequence for a CH3 domain comprising amino acid substitutions that favor formation of heterodimer over homodimer, in a suitable manner, so as to provide a binding protein of the disclosure or a nucleotide sequence or nucleic acid encoding the same.

### Affinity Maturation

In designing binding proteins for therapeutic applications, it is desirable to create proteins that, for example, modulate a functional activity of a target, and/or improved binding proteins such as binding proteins with higher specificity and/or affinity and/or and binding proteins that are more bioavailable, or stable or soluble in particular cellular or tissue environments.

The binding proteins described in the present disclosure exhibit improved the binding affinities towards a target, for example a tumor antigen expressed on a cell surface. In some embodiments, the binding proteins of the present disclosure is affinity matured to increase its binding affinity to the target binding domain, using any known technique for affinity-maturation (e.g., mutagenesis, chain shuffling, CDR amino acid substitution). Amino acid substitutions may be conservative or semi-conservative. For example, the amino acids glycine, alanine, valine, leucine and isoleucine can often be substituted for one another (amino acids having aliphatic side chains). Of these possible substitutions, typically glycine and alanine are used to substitute for one another since they have relatively short side chains and valine, leucine and isoleucine are used to substitute for one another since they have larger aliphatic side chains which are hydrophobic. Other amino acids which may often be substituted for one another include but are not limited to: phenylalanine, tyrosine and tryptophan (amino acids having aromatic side chains); lysine, arginine and histidine (amino acids having basic side chains); aspartate and glutamate (amino acids having acidic side chains); asparagine and glutamine (amino acids having amide side chains); and cysteine and methionine (amino acids having sulphur-containing side chains). In some embodiments, the binding proteins are isolated by screening combinatorial libraries, for example, by generating phage display libraries and screening such libraries for antibodies possessing the desired binding characteristics towards a target antigen, such as a tumor antigen expressed on a cell surface.

### Binding Protein Modifications

The binding proteins described herein encompass derivatives or analogs in which (i) an amino acid is substituted with an amino acid residue that is not one encoded by the genetic code, (ii) the mature polypeptide is fused with another compound such as polyethylene glycol, or (iii) additional amino acids are fused to the protein, such as a leader or secretory sequence or a sequence to block an immunogenic domain and/or for purification of the protein.

Typical modifications include, but are not limited to, acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphatidylinositol, cross-linking, cyclization, disulfide bond formation, demethylation, formation of covalent crosslinks, formation of cystine, formation of pyroglutamate, formylation, gamma carboxylation, glycosylation, GPI anchor formation, hydroxylation, iodination, methylation, myristylation, oxidation, proteolytic processing, phosphorylation, prenylation, racemization, selenoylation, sulfation, transfer-RNA mediated addition of amino acids to proteins such as arginylation, and ubiquitination.

Modifications are made anywhere in the -binding proteins described herein, including the peptide backbone, the amino acid side-chains, and the amino or carboxyl termini. Certain common peptide modifications that are useful for modification of the binding proteins include glycosylation, lipid attachment, sulfation, gamma-carboxylation of glutamic acid residues, hydroxylation, blockage of the amino or carboxyl group in a polypeptide, or both, by a covalent modification, and ADP-ribosylation.

In some embodiments, the binding proteins of the disclosure are conjugated with drugs to form antibody-drug conjugates (ADCs). In general, ADCs are used in oncology applications, where the use of antibody-drug conjugates for the local delivery of cytotoxic or cytostatic agents allows for the targeted delivery of the drug moiety to tumors, which can allow higher efficacy, lower toxicity, etc.

### Polynucleotides Encoding Binding Proteins

Also provided, in some embodiments, are polynucleotide molecules encoding a binding protein as described herein. In some embodiments, the polynucleotide molecules are provided as a DNA construct. In other embodiments, the polynucleotide molecules are provided as a messenger RNA transcript.

The polynucleotide molecules are constructed by known methods such as by combining the genes encoding the various domains of the binding proteins, operably linked to a suitable promoter, and optionally a suitable transcription terminator, and expressing it in bacteria or other appropriate expression system such as, for example CHO cells.

In some embodiments, the polynucleotide is inserted into a vector, preferably an expression vector, which represents a further embodiment. This recombinant vector can be constructed according to known methods. Vectors of particular interest include plasmids, phagemids, phage derivatives, virii (e.g., retroviruses, adenoviruses, adeno-associated viruses, herpes viruses, lentiviruses, and the like), and cosmids.

A variety of expression vector/host systems may be utilized to contain and express the polynucleotide encoding the polypeptide of the described binding proteins.

Examples of expression vectors for expression in E. coli are pSKK (Le Gall et al., J Immunol Methods. (2004) 285(1):111-27), pcDNA5 (Invitrogen) for expression in mammalian cells, PICHIAPINK™ Yeast Expression Systems (Invitrogen), BACUVANCE™ Baculovirus Expression System (GenScript).

Thus, the binding proteins as described herein, in some embodiments, are produced by introducing a vector encoding the protein as described above into a host cell and culturing said host cell under conditions whereby the protein domains are expressed, may be isolated and, optionally, further purified.

### Production of Binding Proteins

Disclosed herein, in some embodiments, is a process for the production of a -binding protein of the present disclosure. In some embodiments, the process comprises culturing a host transformed or transfected with a vector comprising a nucleic acid sequence encoding a binding protein under conditions allowing the expression of the binding protein and recovering and purifying the produced protein from the culture.

In an additional embodiment is provided a process directed to improving one or more properties, e.g., affinity, stability, heat tolerance, cross-reactivity, etc., of abinding protein described herein, compared to a reference binding compound. In some embodiments, a plurality of single-substitution libraries is provided each corresponding to a different domain, or amino acid segment of a cbinding protein or reference binding compound such that each member of the single-substitution library encodes only a single amino acid change in its corresponding domain, or amino acid segment. Typically, this allows all of the potential substitutions in a large protein or protein binding site to be probed with a few small libraries. In some embodiments, the plurality of domains forms or covers a contiguous sequence of amino acids of a binding protein or a reference binding compound. Nucleotide sequences of different single-substitution libraries overlap with the nucleotide sequences of at least one other single-substitution library. In some embodiments, a plurality of single-substitution libraries are designed so that every member overlaps every member of each single-substitution library encoding an adjacent domain.

Binding proteins expressed from such single-substitution libraries are separately selected to obtain a subset of variants in each library which has properties at least as good as those of the reference binding compound and whose resultant library is reduced in size. Generally, the number of nucleic acids encoding the selected set of binding compounds is smaller than the number of nucleic acids encoding members of the original single-substitution library. Such properties include, but are not limited to, affinity to a target compound, stability with respect to various conditions such as heat, high or low pH, enzymatic degradation, cross-reactivity to other proteins and the like. The selected compounds from each single-substitution library are referred to herein interchangeably as “pre-candidate compounds,” or “pre-candidate proteins.” Nucleic acid sequences encoding the pre-candidate compounds from the separate single-substitution libraries are then shuffled in a PCR to generate a shuffled library, using PCR-based gene shuffling techniques.

An exemplary work flow of the screening process is described herein. Libraries of pre-candidate compounds are generated from single substitution libraries and selected for binding to the target protein(s), after which the pre-candidate libraries are shuffled to produce a library of nucleic acids encoding candidate compounds which, in turn, are cloned into a convenient expression vector, such as a phagemid expression system. Phage expressing candidate compounds then undergo one or more rounds of selection for improvements in desired properties, such as binding affinity to a target molecule. Target molecules may be adsorbed or otherwise attached to a surface of a well or other reaction container, or target molecules may be derivatized with a binding moiety, such as biotin, which after incubation with candidate binding compounds may be captured with a complementary moiety, such as streptavidin, bound to beads, such as magnetic beads, for washing. In exemplary selection regimens, the candidate binding compounds undergo a wash step so that only candidate compounds with very low dissociation rates from a target molecule are selected. Exemplary wash times for such embodiments are about 10 minutes, about 15 minutes, about 20 minutes, about 20 minutes, about 30 minutes, about 35 minutes, about 40 minutes, about 45 minutes, about 50 minutes, about 55 mins, about 1 hour, about 2 hours, about 3 hours, about 4 hours, about 5 hours, about 6 hours, about 7 hours, about 8 hours; or in other embodiments, about 24 hours; or in other embodiments, about 48 hours; or in other embodiments, about 72 hours. Isolated clones after selection are amplified and subjected to an additional cycle of selection or analyzed, for example by sequencing and by making comparative measurements of binding affinity towards their target, for example, by ELISA, surface plasmon resonance (SPR), bio-layer interferometry (e.g., OCTET® system, Pall Life Sciences, ForteBio, Menlo Park, Calif.) or the like.

### Pharmaceutical Compositions

Also provided, in some embodiments, are pharmaceutical compositions comprising a binding protein described herein, a vector comprising the polynucleotide encoding the polypeptide of a binding protein or a host cell transformed by this vector and at least one pharmaceutically acceptable carrier. The term “pharmaceutically acceptable carrier” includes, but is not limited to, any carrier that does not interfere with the effectiveness of the biological activity of the ingredients and that is not toxic to the patient to whom it is administered. Examples of suitable pharmaceutical carriers are well known in the art and include phosphate buffered saline solutions, water, emulsions, such as oil/water emulsions, various types of wetting agents, sterile solutions etc. Such carriers can be formulated by conventional methods and can be administered to the subject at a suitable dose. Preferably, the compositions are sterile. These compositions may also contain excipients such as preservative, emulsifying agents and dispersing agents. Prevention of the action of microorganisms may be ensured by the inclusion of various antibacterial and antifungal agents.

In some embodiments of the pharmaceutical compositions, a binding protein is encapsulated in nanoparticles. In some embodiments, the nanoparticles are fullerenes, liquid crystals, liposome, quantum dots, superparamagnetic nanoparticles, dendrimers, or nanorods. In other embodiments of the pharmaceutical compositions, the conditionally active multivalent target binding protein is attached to liposomes. In some instances, a binding protein is conjugated to the surface of liposomes. In some instances, a conditionally active multivalent target binding proteinis encapsulated within the shell of a liposome. In some instances, the liposome is a cationic liposome.

The -binding proteins described herein are contemplated for use as a medicament. Administration is effected by different ways, e.g., by intravenous, intraperitoneal, subcutaneous, intramuscular, topical or intradermal administration. In some embodiments, the route of administration depends on the kind of therapy and the kind of compound contained in the pharmaceutical composition. The dosage regimen will be determined by the attending physician and other clinical factors. Dosages for any one patient depends on many factors, including the patient's size, body surface area, age, sex, the particular compound to be administered, time and route of administration, the kind of therapy, general health and other drugs being administered concurrently. An “effective dose” refers to amounts of the active ingredient that are sufficient to affect the course and the severity of the disease, leading to the reduction or remission of such pathology and may be determined using known methods.

### Methods of Treatment

Also provided herein, in some embodiments, are methods and uses for stimulating the immune system of an individual in need thereof comprising administration of a binding protein as described herein. In some instances, the administration of a binding protein induces and/or sustains cytotoxicity towards a cell expressing a target antigen. In some instances, the cell expressing a target antigen is a cancer or tumor cell, a virally infected cell, a bacterially infected cell, an autoreactive T or B cell, damaged red blood cells, arterial plaques, or inflamed or fibrotic tissue cell. In some embodiments, the target antigen is an immune modulatory protein.

Also provided herein are methods and uses for a treatment of a disease, disorder or condition associated with a target antigen comprising administering to an individual in need thereof a binding protein as described herein. Diseases, disorders or conditions associated with a target antigen include, but are not limited to, viral infection, bacterial infection, auto-immune disease, transplant rejection, atherosclerosis, or fibrosis. In some embodiments the viral infection is hepatitis. In other embodiments, the disease, disorder or condition associated with a target antigen is a proliferative disease, a tumorous disease, an inflammatory disease, an immunological disorder, an autoimmune disease, an infectious disease, a viral disease, an allergic reaction, a parasitic reaction, a graft-versus-host disease or a host-versus-graft disease. In one embodiment, the disease, disorder or condition associated with a target antigen is cancer. In one instance, the cancer is a hematological cancer. In another instance, the cancer is a melanoma. In a further instance, the cancer is non-small cell lung cancer. In yet further instance, the cancer is breast cancer. In some embodiments, a binding protein binds to an immune modulatory protein and is administered to treat a cancer characterized by upregulation of said immune modulatory protein. For example, the immune modulatory protein is, in some cases, CTLA-4 and the cancer is melanoma, non-small cell lung cancer, triple negative breast cancer, or ovarian cancer.

As used herein, in some embodiments, “treatment” or “treating” or “treated” refers to therapeutic treatment wherein the object is to slow (lessen) an undesired physiological condition, disorder or disease, or to obtain beneficial or desired clinical results. For the purposes described herein, beneficial or desired clinical results include, but are not limited to, alleviation of symptoms; diminishment of the extent of the condition, disorder or disease; stabilization (i.e., not worsening) of the state of the condition, disorder or disease; delay in onset or slowing of the progression of the condition, disorder or disease; amelioration of the condition, disorder or disease state; and remission (whether partial or total), whether detectable or undetectable, or enhancement or improvement of the condition, disorder or disease. Treatment includes eliciting a clinically significant response without excessive levels of side effects. Treatment also includes prolonging survival as compared to expected survival if not receiving treatment. In other embodiments, “treatment” or “treating” or “treated” refers to prophylactic measures, wherein the object is to delay onset of or reduce severity of an undesired physiological condition, disorder or disease, such as, for example is a person who is predisposed to a disease (e.g., an individual who carries a genetic marker for a disease such as breast cancer).

In some embodiments of the methods described herein, binding proteins described herein are administered in combination with an agent for treatment of the particular disease, disorder or condition. Agents include but are not limited to, therapies involving antibodies, small molecules (e.g., chemotherapeutics), hormones (steroidal, peptide, and the like), radiotherapies (y-rays, X-rays, and/or the directed delivery of radioisotopes, microwaves, UV radiation and the like), gene therapies (e.g., antisense, retroviral therapy and the like) and other immunotherapies. In some embodiments, the conditionally active multivalent target-binding protein described herein are administered in combination with anti-diarrheal agents, anti-emetic agents, analgesics, opioids and/or non-steroidal anti-inflammatory agents. In some embodiments, a conditionally active multivalent target-binding protein described herein are administered before, during, or after surgery.

According to another embodiment of the invention, kits for detecting a cancer, and for diagnosis, prognosis or monitoring are provided. The kits include the foregoing binding proteins (e.g., labeled anti-immune modulatory protein conditionally active multivalent target-binding antibody or antigen binding fragments thereof, a pro immune modulating molecule as descried herein), and one or more compounds for detecting the label. In some embodiments, the label is selected from the group consisting of a fluorescent label, an enzyme label, a radioactive label, a nuclear magnetic resonance active label, a luminescent label, and a chromophore label.

### Additional Embodiments

Embodiment one provides a conditionally active multivalent protein comprising:
a first binding moiety comprising a first non-CDR loop and CDRs, a first cleavable linker, and a first target antigen binding domain capable of binding a first target,
a second binding moiety comprising a second non-CDR loop and CDRs, a second cleavable linker, and a second target antigen binding domain capable of binding a second target, and
a Fc domain;
   wherein the first binding moiety and the second binding moiety are each capable of masking the binding of the first and the second target antigen binding domains to their respective targets.

Embodiment two provides the conditionally active multivalent protein of the previous embodiment, wherein the first binding moiety is capable of binding the first target via the CDRs of the first binding moiety and to the first target antigen binding domain via the first non-CDR loop.

Embodiment three provides the conditionally active multivalent protein of embodiment one or two, wherein the second binding moiety is capable of binding the second target via the CDRs of the second binding moiety and to the second target antigen binding domain via the second non-CDR loop.

Embodiment four provides the conditionally active multivalent protein of embodiment two or three, wherein the first and the second targets are the same or different.

Embodiment five provides the conditionally active multivalent protein of any one of embodiments one through four, wherein the first or the second binding moiety is a natural peptide, a synthetic peptide, an engineered scaffold, or an engineered bulk serum protein.

Embodiment six provides the conditionally active multivalent protein of embodiment five, wherein the engineered scaffold comprises a sdAb, a scFv, a Fab, a VHH, a fibronectin type III domain, immunoglobulin-like scaffold, DARPin, cystine knot peptide, lipocalin, three-helix bundle scaffold, or a DNA or RNA aptamer scaffold.

Embodiment seven provides the conditionally active multivalent protein of any one of embodiments one through six, wherein the first or the second non-CDR loop is a non-CDR loop of a variable domain, a constant domain, a C1 set domain, a C2 set domain, an I-domain, or any combinations thereof.

Embodiment eight provides the conditionally active multivalent protein of any one of embodiments one through seven, wherein the first and the second cleavable linkers each comprise a cleavage site.

Embodiment nine provides the conditionally active multivalent protein of embodiment eight, wherein the cleavage sites are recognized by a protease, are pH sensitive, or are cleaved by chemical degradation.

Embodiment ten provides the conditionally active multivalent protein of embodiment nine, wherein the first binding moiety is covalently linked to the first target antigen binding domain and the second binding moiety is covalently linked to the second target antigen binding domain.

Embodiment eleven provides the conditionally active multivalent protein of any one of embodiments one through ten, wherein upon cleavage of the first and the second cleavable linkers, the first and the second target antigen binding domains bind to their respective targets.

Embodiment twelve provides the conditionally active multivalent protein of any one of embodiments one through eleven, wherein the first binding moiety is capable of masking the binding of the first target antigen binding domain to its target via specific intermolecular interactions between the first binding moiety and the first target antigen binding domain.

Embodiment thirteen provides the conditionally active multivalent protein of any one of embodiments one through 12, wherein the second binding moiety is capable of masking the binding of the second target antigen binding domain to its target via specific intermolecular interactions between the second binding moiety and the second target antigen binding domain.

Embodiment fourteen provides the conditionally active multivalent protein of any one of embodiments one through thirteen, wherein the first or the second target antigen binding domain comprises a sdAb, a scFv, a Fab, or a variable heavy chain domain (VHH).

Embodiment fifteen provides the conditionally active multivalent protein of any of embodiments one through fourteen, wherein the first target antigen binding domain comprises a first variable heavy chain domain (VHH1) and the second target antigen binding domain comprises a second variable heavy chain domain (VHH2).

Embodiment sixteen provides the conditionally active multivalent protein of any one of embodiments one through fifeteen, wherein the first target antigen and the second target antigen are the same antigen and wherein the activated protein binds to two molecules of same antigen.

Embodiment seventeen provides the conditionally active multivalent protein of any one of embodiments one through sixteen, wherein the first target antigen and the second target antigen are the same antigen and wherein the activated protein binds to two epitopes of the same antigen.

Embodiment eighteen provides the conditionally active multivalent protein of any one of embodiments one through fifteen, wherein the first target antigen and the second target antigen are different antigens.

Embodiment nineteen provides the conditionally active multivalent protein of any one of embodiments one through eighteen, wherein the first or the second target binds to a tumor antigen.

Embodiment twenty provides the conditionally active multivalent protein of embodiment 19, wherein the tumor antigen comprises EGFR, PSMA, EpCAM, BCMA, 5T4, AFP, Axl, B7-H3, Cadherin-6, CAIX, CD117, CD123, CD138, CD166, CD19, CD20, CD205, CD22, CD30, CD33, CD352, CD37, CD38, CD44, CD52, CD56, CD70, CD71, CD74, CD79b, CEACAM5, c-MET, DLL3, EphA2, FAP, FGFR2, FGFR3, glypican-3, FLT-3, FOLR1, gpNMB, HER2, HPV-16 E6, HPV-16 E7, ITGA3, SLC39A6, Mesothelin, Muc1, Muc16, NaPi2b, Nectin-4, P-cadherin, Prolactin R, PSCA, PTK7, ROR1, SLC44A4, SLTRK5, SLTRK6, STEAP1, TIM1, Trop2, or WT1.

Embodiment twenty-one provides the conditionally active multivalent protein of any one of embodiments one through twenty, wherein the first or the second target comprises an immune modulatory protein.

Embodiment twenty-two provides the conditionally active multivalent protein of claim embodiment twenty-one, wherein the immune modulatory protein comprises CTLA-4, CD27, CD137, 2B4, TIGIT, CD155, ICOS, HVEM, CD40L, LIGHT, TIM-1, OX40, DNAM-1, PD-L1, PD1, PD-L2, CD8, CD40, CEACAM1, CD48, CD70, A2AR, CD39, CD73, B7-H3, B7-H4, BTLA, IDOL IDO2, TDO, KIR, LAG-3, TIM-3, VISTA, IL6-R, IL-6, TNFα, CD19, CD20, CD22, CD52, integrin a4, integrin a4b7, CD11a, CTLA4-Ig fusion, IL-17, IL12/23, IL12, IL23, or TGF-beta.

Embodiment twenty-three provides the conditionally active multivalent protein of any one of embodiments one through twenty-two, wherein the first or the second target antigen binding domain binds to an immune cell.

Embodiment twenty-four provides the conditionally active multivalent protein of any one of embodiments one through twenty-two, wherein the first or the second target antigen binding domain binds to a T-cell.

Embodiment twenty-five provides the conditionally active multivalent protein of any one of embodiments one through twenty-two, wherein the first or the second target antigen binding domain binds to CD3.

Embodiment twenty-six provides the conditionally active multivalent protein of any one of embodiments one through twenty-five, wherein the first and the second cleavable linkers each comprise protease cleavage sites.

Embodiment twenty-seven provides the conditionally active multivalent protein of embodiment twenty-six, wherein the protease cleavage site is recognized by one of a serine protease, a cysteine protease, an aspartate protease, a threonine protease, a glutamic acid protease, a metalloproteinase, a gelatinase, and a asparagine peptide lyase.

Embodiment twenty-eight provides the conditionally active multivalent protein of embodiment twenty-six, wherein the protease cleavage site is recognized by one of a Cathepsin B, a Cathepsin C, a Cathepsin D, a Cathepsin E, a Cathepsin K, a Cathepsin L, a kallikrein, a hK1, a hK10, a hK15, a plasmin, a collagenase, a Type IV collagenase, a stromelysin, a Factor Xa, a chymotrypsin-like protease, a trypsin-like protease, a elastase-like protease, a subtilisin-like protease, an actinidain, a bromelain, a calpain, a caspase, a caspase-3, a Mir1-CP, a papain, a HIV-1 protease, a HSV protease, a CMV protease, a chymosin, a renin, a pepsin, a matriptase, a legumain, a plasmepsin, a nepenthesin, a metalloexopeptidase, a metalloendopeptidase, a matrix metalloprotease (MMP), a MMP1, a MMP2, a MMP3, a MMP8, a MMP9, a MMP10, a MMP11, a MMP12, a MMP13, a MMP14, an ADAM10, an ADAM12, an urokinase plasminogen activator (uPA), an enterokinase, a prostate-specific target (PSA, hK3), an interleukin-1β converting enzyme, a thrombin, a FAP (FAP-α), a type II transmembrane serine protease (TTSP), a neutrophil elastase, a cathepsin G, a proteinase 3, a neutrophil serine protease 4, a mast cell chymase, a mast cell tryptase, a dipeptidyl peptidase, and a dipeptidyl peptidase IV (DPPIV/CD26).

Embodiment twenty-nine provides the conditionally active multivalent protein of any one of embodiments one through twenty-eight, wherein the Fc domain is capable of extending the half-life of the protein.

Embodiment thirty provides the conditionally active multivalent protein of any one of embodiments one through twenty-nine, wherein the Fc domain comprises an antibody, a sdAb-Fc, or a scFv-Fc.

Embodiment thirty provides the conditionally active multivalent protein of any one of embodiments one through thirty, further comprising a first target bound to the first binding moiety and a second target bound to the second binding moiety, wherein the first and the second targets provide the multivalent protein with a systemic pharmacological activity, and wherein upon cleavage of the first and the second cleavable linkers the multivalent protein is activated by separation of the first and the second binding moiety and the first and the second targets, from the first and the second target antigen binding domains, respectively, and the binding protein is thereby activated for local binding with first and second target antigens.

Embodiment thirty-two provides the conditionally active multivalent protein of embodiment thirty-one, wherein the cleavage of the first and the second cleavable linkers are in a tumor microenvironment.

Embodiment thirty-three provides the conditionally active multivalent protein of embodiment 31 or embodiment 32, wherein the first and the second targets are PD-L1, and wherein the first and the second target antigens are CTLA4.

Embodiment thirty-four provides a conditionally active multivalent protein, comprising:
a first binding moiety bound to a first target antigen binding domain by a first non-CDR loop, bound to a first target domain by CDRs, wherein the first binding moiety and the first target antigen binding domains are connected by a first cleavable linker
a second binding moiety bound to a first target antigen binding domain by a second non-CDR loop, bound to a second target domain by CDRs, wherein the second binding moiety and the second target antigen binding domains are connected by a second cleavable linker; and
an Fc domain;
   wherein the first and the second target antigen binding domains are capable of binding to a first target antigen and a second target antigen, respectively,
   wherein the multivalent protein has a systemic pharmacological activity prior to its activation by cleavage of the first and the second cleavable linkers, and wherein upon activation the first and the second targets and the first and the second binding moieties are separated from the first and the second target antigen binding domains, and
   wherein the multivalent protein, in its activated state, binds locally with the first and second target antigens.

Embodiment thirty-five provides the conditionally active multivalent protein of embodiment thirty-four, wherein the cleavage of the first and the second cleavable linkers is in a tumor microenvironment.

Embodiment thirty-six provides the conditionally active multivalent protein of embodiment thirty-four or embodiment thirty-five, wherein the first and the second targets are PD-L1, and wherein the first and the second target antigens are CTLA4.

### EXAMPLES

The examples below further illustrate the described embodiments without limiting the scope of the invention.

### Example 1

### Treatment With an Exemplary Antibody of the Present Disclosure Inhibits In Vivo Tumor Growth

Murine tumor line CT26 is implanted subcutaneously in Balb/c mice and on day 7 post-implantation the average size of the tumor is measured. Mice are treated with an exemplary conditionally active multivalent CTLA-4 binding antibody according to the present disclosure, or a control CTLA-4 binding antibody which is not conditionally active. Results show that treatment with the exemplary conditionally active multivalent CTLA-4 binding antibody according to the present disclosure inhibits tumor growth to a greater extent than the control antibody.

### Example 2

### Exemplary Antibody of the Present Disclosure Exhibits Reduced Specificity Towards Cell Line Which Overexpresses Antigens but is Protease Deficient

Cells overexpressing CTLA-4 and exhibiting low expression of a matrix metalloprotease are separately incubated with an exemplary conditionally active multivalent CTLA-4 binding antibody according to the present disclosure, or a control CTLA-4 binding antibody which is not conditionally active. Cells expressing normal levels of antigens and proteases are also incubated with the exemplary CTLA-4 binding antibody according to the present disclosure, or a control CTLA-4 binding antibody.

Results indicate that in the absence of protease secretion, the conditionally active antibody of the present disclosure binds the protease expressing cells but does not bind the protease-deficient antigen expressing cells. In contrast, the control antibody lacks the ability to selectively bind the protease expressing cells over the protease deficient ones. Thus, the exemplary conditionally active multivalent antibody of the present disclosure is advantageous, for example, in terms of reducing off-tumor toxicity.

### Example 3

### Construction of an Exemplary Conditionally Active Multivalent Target Binding Protein Which Comprises a Binding Moiety That Binds to PD-L1 and a Target Antigen Binding Domain Whose Target is CTLA4

The sequence of an engineered protein scaffold comprising CDR loops capable of binding PD-L1 and non-CDR loops is obtained. Overlapping PCR is used to introduce random mutations in the non-CDR loop regions, thereby generating a library. The resultant sequences are cloned into a phage display vector, thereby generating a phage display library. Escherichia coli cells are transformed with the library and used to construct a phage display library. ELISA is performed using an immobilized target antigen binding domain with specificity for CTLA4. A clone with high specificity for CTLA4 is selected. Affinity maturation is performed by re-randomizing residues in the non-CDR loop regions as before.

Sequence alignment of non-CDR loop regions of the resultant proteins is performed to determine sequence conservation between proteins with high affinity for the CTLA4 binding target antigen binding domain. Site directed mutagenesis of one or more amino acids within these regions of sequence conservation is performed to generate additional proteins. Binding of the resultant proteins to an immobilized target antigen binding domain whose target is CTLA4 is measured in an ELISA. A protein with the highest affinity for the target antigen binding domain is selected.

The sequence of this binding moiety is cloned into a vector comprising a sequence for a cleavable linker, and sequences for a second binding moiety that has non-CDR loops specific for a second target antigen binding domain that is same or different as the first target antigen binding domain, i.e., CTLA4, and a Fc domain. The resultant vector is expressed in a heterologous expression system to obtain a conditionally active multivalent target binding protein that comprises a binding moiety comprising a cleavable linker and non-CDR loops which provide a binding site specific for the target antigen binding domain whose target is CTLA4, and CDR loops which are specific for PD-L1.

### Example 4

### Construction of an Exemplary Conditionally Active Multivalent Target Binding Protein Which Comprises a Binding Moiety That Binds to PD-L1 and a Target Antigen Binding Domain Whose Target is EGFR

The sequence of an engineered protein scaffold comprising CDR loops capable of binding PD-L1 and non-CDR loops is obtained. Overlapping PCR is used to introduce random mutations in the non-CDR loop regions, thereby generating a library. The resultant sequences are cloned into a phage display vector, thereby generating a phage display library. Escherichia coli cells are transformed with the library and used to construct a phage display library. ELISA is performed using an immobilized target antigen binding domain with specificity for EGFR. A clone with high specificity for EGFR is selected. Affinity maturation is performed by re-randomizing residues in the non-CDR loop regions as before.

Sequence alignment of non-CDR loop regions of the resultant proteins is performed to determine sequence conservation between proteins with high affinity for the EGFR binding target antigen binding domain. Site directed mutagenesis of one or more amino acids within these regions of sequence conservation is performed to generate additional proteins. Binding of the resultant proteins to an immobilized target antigen binding domain whose target is EGFR is measured in an ELISA. A protein with the highest affinity for the target antigen binding domain is selected.

The sequence of this binding moiety is cloned into a vector comprising a sequence for a cleavable linker, and sequences for a second binding moiety that has non-CDR loops specific for a second target antigen binding domain that is same or different as the first target antigen binding domain, i.e., EGFR, and a Fc domain. The resultant vector is expressed in a heterologous expression system to obtain a conditionally active multivalent target binding protein that comprises a binding moiety comprising a cleavable linker and non-CDR loops which provide a binding site specific for the target antigen binding domain whose target is EGFR, and CDR loops which are specific for PD-L1.

### Example 5

### Construction of an Exemplary Dual Binding Moiety Which Binds to Albumin (Anti-Albumin) and a Target Antigen Binding Domain Whose Target is CD40 (Anti-CD40)

Engineered protein scaffolds comprising CDR loops capable of binding albumin and non-CDR loops capable of binding an anti-CD40 antibody were obtained. Overlapping PCR was used to introduce random mutations in the non-CDR loop regions of the engineered protein scaffolds, generating a library. The resultant sequences were cloned into a phage display vector and transformed into Escherichia coli cells to express a phage display library. Panning was performed using an immobilized target antigen binding domain with specificity for CD40 (anti-CD40).

The sequences from this panning were subsequently cloned into an expression vector, in an expression construct comprising a signal domain followed by an anti-Albumin binding domain containing one of the selected non-CDR loop mutations (SEQ ID Nos. 48-60) followed by a (GGGGSGGGS)₂ linker (SEQ ID No. 61) followed by an anti-CD40 antibody light chain (SEQ ID No. 72), to generate anti-Alb masked anti-CD40 constructs. An unmasked control construct was also created comprising a signal domain followed by a wild type anti-Albumin binding domain (SEQ ID No. 62) followed by a (GGGGSGGGS)₂ linker (SEQ ID No. 61) followed by an anti-CD40 antibody light chain (SEQ ID No. 72). These constructs were transfected into EXPI293™ cells along with an anti-CD40 heavy chain (SEQ ID No. 73) to generate anti-Alb masked anti-CD40 proteins. The amount of anti-Alb masked anti-CD40 protein in the conditioned media from the transfected EXPI293™ cells was quantitated using an Octet instrument with anti-huFc tips. An antibody of similar molecular weight was used as a standard.

Using conditioned media containing known concentrations of masked anti-CD40 proteins, the binding response of the masked anti-CD40 proteins toward human CD40 protein was measured with an Octet instrument, using a method where the CD40 protein was immobilized (see SEQ ID No. 92, C00703) on biolayer interferometry sensors and the masked anti-CD40 proteins were in solution. The measurements were made using a single 100 nM concentration of the masked anti-CD40 proteins, which allowed for rank ordering based on binding response. The binding response, measured as a nm shift in the biolayer interferometry signal, was compared to the unmasked control anti-Alb-anti-CD40 (Table 2). The mask sequences were found to inhibit CD40 binding to varying extents, with the best mask achieving binding responses of 7% of the unmasked control protein.

TABLE 2
Octet binding responses of anti-Alb masked anti-
CD40 proteins binding to immobilized huCD40

| | Binding response |
|---|---|
| Mask | (% of control) |
| anti-Alb-CD-2-C06 (mask070) | 7% |
| anti-Alb-CD-2-C05 | 31% |
| anti-Alb-CD-2-D06 | 42% |
| anti-Alb-CD-2-D07 | 48% |
| anti-Alb-CD-1-F06 | 57% |
| anti-Alb-CD-2-C11 | 63% |
| anti-Alb-CD-2-F10 | 63% |
| anti-Alb-CD-2-B07 | 64% |
| anti-Alb-CD-2-A10 | 65% |
| anti-Alb-CD-2-D09 | 66% |
| anti-Alb-CD-1-A09 | 67% |
| anti-Alb-CD-2-D08 | 68% |
| anti-Alb-CD-2-F09 | 75% |

### Example 6

### Binding of Masked Molecules to huCD40

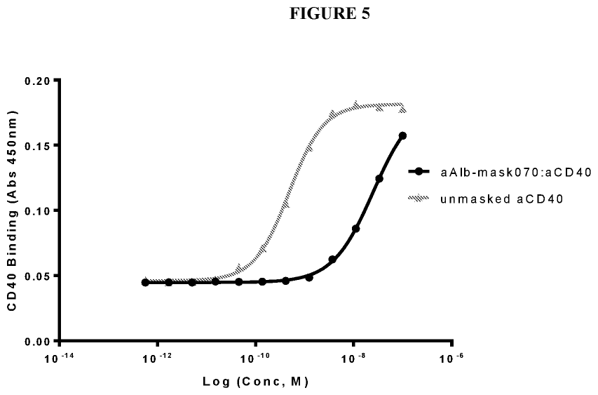

An exemplary anti-Alb masked anti-CD40 protein (mask070, SEQ ID No. 80) and an unmasked control (SEQ ID No. 72) were expressed along with anti-CD40 heavy chain (SEQ ID No C2764) in EXPI293™ cells and purified via Protein A chromatography. The proteins were then tested in an ELISA assay for binding to immobilized huCD40 and the results are shown in FIG. 5 and summarized in Table 3. The anti-Alb mask decreased the binding of the protein to human CD40 by 52 fold.

TABLE 3
Binding of exemplary anti-Alb masked anti-CD40
protein and an unmasked control to human CD40

| | | Binding |
|---|---|---|
| Mask | EC₅₀ (nM) | shift |
| anti-Alb mask070 anti-CD40 | 25 | 52 |
| C2882, C2764 (aCD40 | | |
| Heavy Chain GDSE) | | |
| Unmasked anti-CD40 | 0.48 | 1 |
| C00697, C2764 (aCD40 | | |
| Heavy Chain GDSE) | | |

### Example 7

### Potency of Exemplary Masked Anti-CD40 Proteins in a B Cell Activation Assay

The exemplary anti-Alb masked anti-CD40 proteins were tested for their ability to activate human B cells. The anti-Alb masked anti-CD40 proteins were cloned with a non-cleavable linker (SEQ ID No. 80) and a protease cleavable linker (SEQ ID No. 90) between the anti-Alb domain and the anti-CD40 light chain. A control active mimic construct was also made containing the anti-CD40 light chain without an anti-Alb domain (SEQ ID No. 72). The constructs were expressed along with anti-CD40 heavy chain (SEQ ID No. 74) in EXPI293™ cells and purified via Protein A chromatography. Prior to the assay, the protein with the protease cleavable linker was treated with protease for 2 hours at 37 ° C., resulting in full cleavage of the anti-Alb mask from the antibody as analyzed by SDS-PAGE.

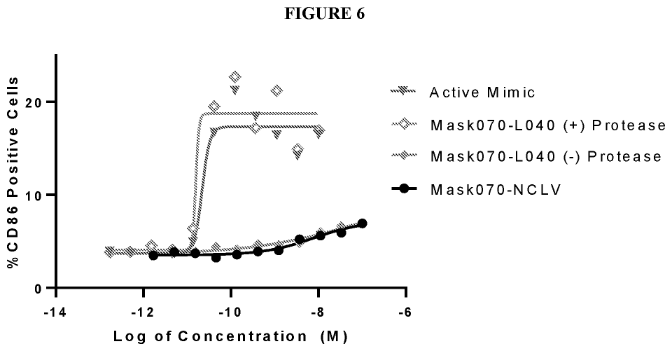

To quantify the ability of the proteins to activate B cells, the proteins were cultured with purified human B cells for 48 hours. To measure activation, the percentage of B cells positive for expression of CD86 was measured using flow cytometry and the results are shown in FIG. 6 and summarized in Table 4. The protease activated protein and the control active mimic had an EC₅₀ of 16 and 21 pM, respectively. The masked proteins did not exhibit enough activity in this assay to quantify, resulting in greater than a 2000 fold shift in activity between the masked and activated proteins.

TABLE 4
B cell activation by masked (mask070)
and activated anti-CD40 proteins

| | | Activity |
|---|---|---|
| Mask | EC₅₀ (M) | shift |
| NCLV | Indeterminate | >2000 |
| C2882 | | |
| (SEQ ID No. 80), | | |
| C2764 | | |
| (SEQ ID No. 74) | | |
| L040 (−) | Indeterminate | >2000 |
| Protease | | |
| C2905, C2764 | | |
| L040 (+) | 1.6E−11 | 1 |
| Protease | | |
| C2905, C2764 | | |
| Active mimic | 2.1E−11 | 1 |
| C00697, C2764 | | |

### Example 8

### Construction of an Exemplary Dual Binding Moiety Which Binds to Albumin (Anti-Albumin) and a Target Antigen Binding Domain Whose Target is CTLA4 (Anti-CTLA4)

Engineered protein scaffolds comprising CDR loops capable of binding albumin and non-CDR loops capable of binding anti-CTLA4 were obtained. Overlapping PCR was used to introduce random mutations in the non-CDR loop regions, generating a library. The resultant sequences were cloned into a phage display vector and transformed into Escherichia coli cells to express a phage display library. Panning was performed using an immobilized target antigen binding domain with specificity for CTLA4 (anti-CTLA4).

The sequences from this panning were subsequently cloned into an expression vector, in an expression construct comprising a signal domain followed by an anti-Albumin binding domain containing the selected non-CDR loop mutations (SEQ ID Nos. 63-71) followed by a (GGGGSGGGS)₂ linker (SEQ ID No. 61) followed by an anti-CTLA4 antibody light chain (SEQ ID No. 78), to generate anti-Alb masked anti-CTLA4 constructs. An unmasked control construct was also created comprising a signal domain followed by a wild type anti-Albumin binding domain (SEQ ID No. 61) followed by a (GGGGSGGGS)₂ linker (SEQ ID No. 61) followed by an anti-CTLA4 antibody light chain (SEQ ID No. 78). These constructs were transfected into EXPI293™ cells along with an anti-CTLA4 heavy chain (SEQ ID No. 79) to generate anti-Alb masked anti-CTLA4 proteins. The amount of anti-Alb masked anti-CTLA4 protein in the conditioned media from the transfected EXPI293™ cells was quantitated using an Octet instrument with anti-huFc tips. An antibody of similar molecular weight was used as a standard.

Using conditioned media containing known concentrations of masked anti-CTLA4 proteins, the binding response of the masked anti-CTLA4 proteins toward human CTLA4 protein was measured with an Octet instrument, using a method where the CTLA4 protein was immobilized (see SEQ ID No. 93, C00705) on biolayer interferometry sensors and the masked anti-CTLA4 proteins were in solution. The measurements were made using a single 100 nM concentration of the masked anti-CTLA4 proteins, which allowed for rank ordering based on binding response. The binding response, measured as a nm shift in the biolayer interferometry signal, was compared to the unmasked control anti-Alb-anti-CTLA4 (Table 5). The mask sequences were found to inhibit CTLA4 binding to varying extents, with the best masks achieving binding responses of 35% and 52% of the unmasked control protein.

TABLE 5
Octet binding responses of anti-Alb masked anti-
CTLA4 proteins binding to immobilized huCTLA4

| | Binding response |
|---|---|
| Mask | (% of control) |
| anti-Alb-CT-A5 | 35% |
| (mask078) | |
| (SEQ ID No. 63) | |
| anti-Alb-CT-A11 | 52% |
| (mask071) | |
| (SEQ ID No. 64) | |
| anti-Alb-CT-B9 | 84% |
| (SEQ ID No. 65) | |
| anti-Alb-CT-B3 | 92% |
| (SEQ ID No. 66) | |
| anti-Alb-CT-B1 | 93% |
| (SEQ ID No. 67) | |
| anti-Alb-CT-A10 | 94% |
| (SEQ ID No. 68) | |
| anti-Alb-CT-A4 | 100% |
| (SEQ ID No. 69) | |
| anti-Alb-CT-A7 | 107% |
| (SEQ ID No. 70) | |
| anti-Alb-CT-D5 | 111% |
| (SEQ ID No. 71) | |

### Example 9

### Binding of Masked Molecules to huCTLA4

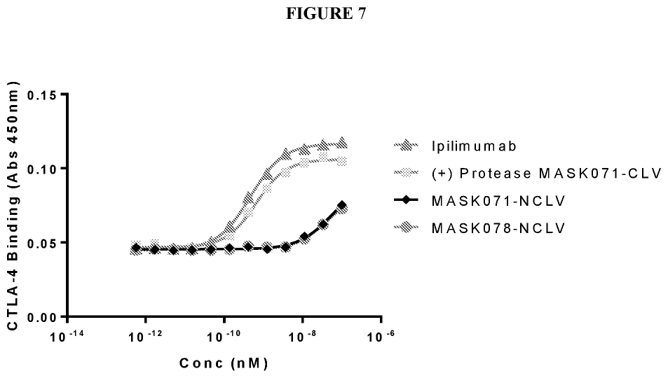

The exemplary anti-Alb masked anti-CTLA4 proteins with non-cleavable linkers (mask071, SEQ ID No. 81; mask078, SEQ ID No. 82), a cleavable linker (mask071 L040, SEQ ID No. 91) and an unmasked control (SEQ ID No. 78) were expressed along with anti-CTLA4 heavy chain (SEQ ID No. 79) in EXPI293™ cells and purified via Protein A chromatography. The proteins were then tested in an ELISA assay for binding to immobilized huCTLA4 and the results are shown in FIG. 7 and summarized in Table 6. The anti-Alb masks decreased the binding of the protein to human CTLA4 by 170 fold and 210 fold for mask071 and mask078, respectively.

TABLE 6
Binding of exemplary anti-Alb masked anti-CTLA4
protein and an unmasked control to human CTLA4

| | | | EC₅₀ | Binding |
|---|---|---|---|---|
| C# | Description | Linker | (nM) | Shift |
| C00826, | aCTLA4 | | 0.47 | |
| C00827 | | | | |
| C00826, | aAlb-m071::aCTLA4 + | L040 | 0.62 | 1.2 |
| C2892 | Protease | | | |
| C00826, | aAlb-m071::aCTLA4 | NCLV | 80 | 170 |
| C2888 | | | | |
| C00826, | aAlb-m078::aCTLA4 | NCLV | 95 | 210 |
| C3268 | | | | |

### Example 10

### Construction of an Eemplary Bnding Miety Wich Bnds to PSMA (Anti-PSMA) and a Target Antigen Binding Domain Whose Target is CTLA4 (Anti-CTLA4)

Engineered protein scaffolds comprising CDR loops capable of binding PSMA and non-CDR loops capable of binding anti-CTLA4 were obtained. The non-CDR loops from the two best mask sequences identified in Example 8 were grafted onto an anti-PSMA binding moiety. The sequences were cloned into an expression construct comprising an anti-PSMA binding domain containing the selected non-CDR loop mutations followed by a flexible linker followed by an anti-CTLA4 antibody light chain to generate anti-PSMA masked anti-CTLA4 constructs (SEQ ID No. 83, SEQ ID No. 84). The constructs were transfected into EXPI293™ cells along with an anti-CTLA4 heavy chain (SEQ ID No. 79) to generate anti-PSMA masked anti-CTLA4 proteins, and the expressed proteins were purified via Protein A chromatography. The anti-PSMA masked anti-CTLA4 proteins were screened for their ability to bind to immobilized human CTLA4 as described in Example 8 and the result is show in Table 7. The anti-PSMA masks inhibited CTLA4 binding, achieving binding responses of 13-28% of the unmasked control protein.

### Example 11

### Construction of an Exemplary Binding Moiety Which Binds to PD1 (Anti-PD1) and a Target Antigen Binding Domain Whose Target is CTLA4 (Anti-CTLA4)

Engineered protein scaffolds comprising CDR loops capable of binding PD1 and non-CDR loops capable of binding anti-CTLA4 were obtained. The non-CDR loops from the sequence anti-Alb-CT-A5 (mask078) (SEQ ID No. 63) identified in Example 8 were grafted onto an anti-PD1 binding moiety. The sequence was cloned into an expression construct comprising an anti-PD1 binding domain containing the selected non-CDR loop mutations followed by a flexible linker followed by an anti-CTLA4 antibody light chain to generate anti-PD1 masked anti-CTLA4 proteins (SEQ ID No. 85). This construct was transfected into EXPI293™ cells along with an anti-CTLA4 heavy chain (SEQ ID No. 79) to generate anti-PD1 masked anti-CTLA4 protein, and the expressed protein was purified via Protein A chromatography. The anti-PD1 masked anti-CTLA4 protein was screened for its ability to bind to immobilized human CTLA4 as described in Example 8 and the result is show in Table 7. The anti-PD1 mask inhibited CTLA4 binding, achieving a binding response of 44% of the unmasked control protein.

TABLE 7
Octet binding responses of anti-PSMA and anti-
PD1 masked anti-CTLA4 proteins binding to huCTLA4

| | Binding response |
|---|---|
| Sequence | (% of control) |
| C00826 (SEQ ID No. 79), | 28% |
| C3280 anti-PSMA-mask071 | |
| (SEQ ID No. 83) | |
| C00826 (SEQ ID No. 79), | 13% |
| C3281 anti-PSMA-mask078 | |
| (SEQ ID No. 84) | |
| C00826 (SEQ ID No. 79), | 44% |
| C3282 anti-PD1-mask078 | |
| (SEQ ID No. 85) | |

### Example 12

### Exemplary Anti-Alb, Anti-PSMA, and Anti-PD1 masked Anti-CTLA4 Proteins Binding to Human Albumin, PSMA, and PD1

Exemplary Anti-Alb (mask071 C2888 (SEQ ID No. 81), mask078 C3268 (SEQ ID No. 82)), anti-PSMA (mask071 C3280 (SEQ ID No. 83), mask078 C3281 (SEQ ID No. 84)), and anti-PD1 (mask078 C3282 (SEQ ID No. 85)) masked anti-CTLA4 proteins were expressed along with anti-CTLA4 heavy chain (SEQ ID No C00826 (SEQ ID No. 79)) in EXPI293™ cells and purified via Protein A chromatography. The proteins were then tested via biolayer interferometry for binding to human albumin, human PSMA, and human PD1. Biotinylated human albumin and biotinylated human PSMA were immobilized (see SEQ ID No. 94, C00651) on streptavidin biosensors and the anti-Alb and anti-PSMA proteins were in solution at 100 nM. The anti-PD1 protein was immobilized on anti-human Fc biosensors and human PD1 was in solution at 300 nM. As shown in FIG. 7, the addition of the mask to the non-CDR loops of the anti-Alb, anti-PSMA, and anti-PD1 single domain antibodies did not prohibit the proteins from binding to their targets.

### Example 13

### CTLA4 ADCC Bioassay Using Exemplary Masked Anti-CTLA4 Proteins

A CTLA4 ADCC bioassay (Promega Cat. # JA3005 and # G7015) was used to measure the potency of masked and unmasked anti-CTLA4 proteins. The bioluminescent cell-based assay involves co-culturing engineered Jurkat cells expressing human CTLA4 as target cells and engineered Jurkat cells expressing the FcγRIIIa receptor, V158 (high affinity) variant, and an NFAT response element driving expression of firefly luciferase as effector cells. Antibody biological activity in ADCC mechanism of action is quantified through the luciferase produced as a result of NFAT pathway activation.

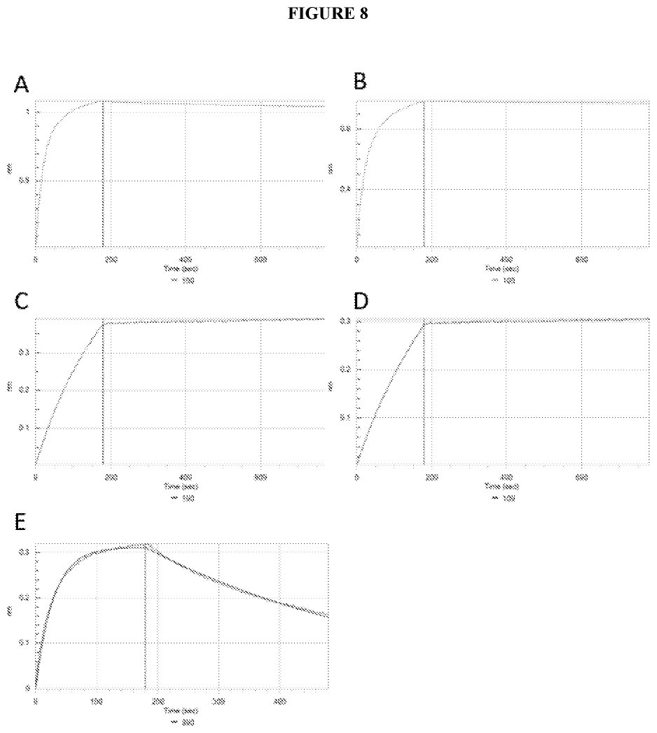

Two masked non-cleavable anti-CTLA4 proteins, one masked cleavable anti-CTLA4 protein, and one protease activated masked anti-CTLA4 protein were tested for their ability to induce ADCC. The anti-CTLA4 proteins were incubated with the CTLA4 target cells and FcγRIIIa effector cells for 6 hours in the presence of 15 g/L human serum albumin. The luminescence was then quantified, and the results are shown in FIG. 8 and summarized in Table 8. The protease activated protein had an EC₅₀ of 20 pM. The masked proteins did not exhibit enough activity in this assay to quantify, resulting in greater than a 5000 fold shift in activity between the masked and activated proteins.

TABLE 8
Potency of various exemplary masked
and unmasked Anti-CTLA4 proteins

| | | | | EC50 | |
|---|---|---|---|---|---|
| PL# | C# | Description | Linker | (M) | Shift |
| PL1378- | C00826 | aAlb- | L040 | 2E−11 | 1 |
| A | (SEQ ID | m071::aCTLA4 + | | | |
| | No. 79), | Protease | | | |
| | C2892 | | | | |
| | (SEQ ID | | | | |
| | No. 91) | | | | |
| PL1378 | C00826 | aAlb- | L040 | 9E−08 | 5000 |
| | (SEQ ID | m071::aCTLA4 | | | |
| | No. 79), | | | | |
| | C2892 | | | | |
| | (SEQ ID | | | | |
| | No. 91) | | | | |
| PL1377 | C00826 | aAlb- | NCLV | >1E7 | >5500 |
| | (SEQ ID | m071::aCTLA4 | | | |
| | No. 79), | | | | |
| | C2888 | | | | |
| | (SEQ ID | | | | |
| | No. 81) | | | | |
| PL1379 | C00826 | aAlb- | NCLV | >1E7 | >5500 |
| | (SEQ ID | m078::aCTLA4 | | | |
| | No. 79), | | | | |
| | C3268 | | | | |
| | (SEQ ID | | | | |
| | No. 82) | | | | |

### Example 14

### CTLA4 Blockade Bioassay using Exemplary Masked Anti-CTLA4 Proteins

A CTLA4 blockade bioassay (Promega Cat. # JA3005) was used to measure the potency of masked and unmasked anti-CTLA4 proteins. The bioluminescent cell-based assay involves co-culturing Jurkat T cells expressing human CTLA4 and a luciferase reporter driven by a native promoter which responds to TCR/CD28 activation (CTLA4 effector cells) and Raji cells expressing an engineered cell surface protein designed to activate cognate TCRs in an antigen-independent manner and endogenously expressing CTLA4 ligands CD80 and CD86 (APC/Raji cells). When the two cell types are co-cultured, CTLA4 competes with CD28 for their shared ligands, CD80 and CD86, and thus inhibits CD28 pathway activation and promoter-mediated luminescence. Addition of an anti-CTLA4 antibody blocks the interaction of CTLA4 with its ligands CD80 and CD86 and results in promoter-mediated luminescence.

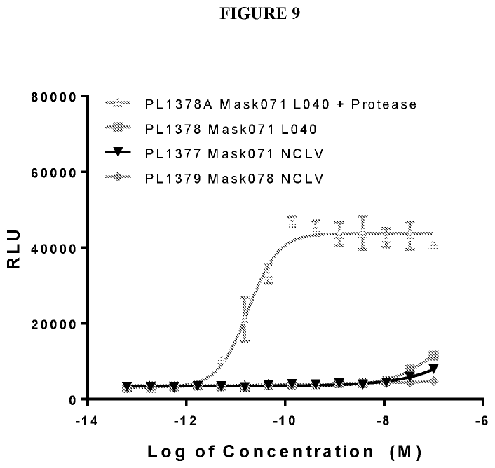

Five exemplary masked anti-CTLA4 proteins, one protease activated masked anti-CTLA4 protein, and one control unmasked anti-CTLA4 protein were tested for their ability to functionally block CTLA4 signaling. The anti-CTLA4 proteins were incubated with the CTLA4 effector cells and Raji cells for 16 hours. The luminescence was then quantified, and the results are shown in FIG. 9 and summarized in Table 9. All five exemplary masked anti-CTLA4 proteins were not active, demonstrating functional masking. Protease treatment of a masked anti-CTLA4 resulted in similar potency to the unmasked control (EC₅₀ of 1.1 nM compared to EC₅₀ of 0.9 nM for the unmasked control).

TABLE 9
Potency of masked and nonmasked anti-CTLA4
proteins in a CTLA4 blockade bioassay

| Sequence | EC₅₀ (M) |
|---|---|
| C00826 (SEQ ID No. 79), C00827 anti- | 8.80E−10 |
| CTLA4 (SEQ ID No. 78) PL538 | |
| C00826 (SEQ ID No. 79), C2892 anti- | 1.10E−09 |
| Alb-m71 (SEQ ID No. 91) (+)Protease | |
| PL1259-A | |
| C00826 (SEQ ID No. 79), C2888 anti- | Not active |
| Alb-m71 (SEQ ID No. 81) PL1256 | |
| C00826 (SEQ ID No. 79), C3280 anti- | Not active |
| PSMA-m71 (SEQ ID No. 83) PL1294 | |
| C00826 (SEQ ID No. 79), C3268 anti- | Not active |
| Alb-m78 (SEQ ID No. 82) PL1291 | |
| C00826 (SEQ ID No. 79), C3281 anti- | Not active |
| PSMA-m78 (SEQ ID No. 84) PL1295 | |
| C00826 (SEQ ID No. 79), C3282 anti- | Not active |
| PD1-m78 (SEQ ID No. 85) PL1296 | |

### Example 15

### Engineering Half-Life Differential Between Intact Pro and Activated Proteins

To prevent accumulation of activated protein and improve the safety of Pro immune modulating molecules, the half-life of the active molecule was engineered to be shorter than that of the intact Pro molecule. To shorten the half-life of the active molecule, the Fc region of an exemplary pro molecule was engineered for reduced FcRn binding by introducing H310A and H435A mutations. To half-life extend the intact Pro molecule, the anti-Alb masks were used to bind and be recycled with human serum albumin.

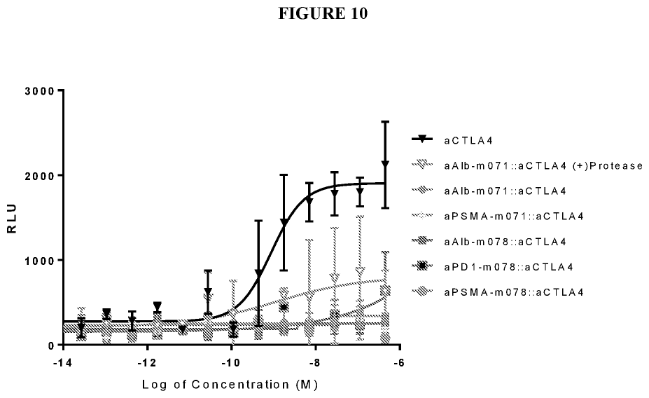

Six anti-CD40 proteins: three different anti-CD40 IgG2 heavy chains containing mutations in the Fc region (wildtype C00696 (SEQ ID No. 75); H310A C2809 (SEQ ID No. 76); H310A+H435A C2849 (SEQ ID No. 77)) combined with two different anti-CD40 light chains either with anti-Alb at the N terminus (C2909) or without anti-Alb at the N terminus (C00697 (SEQ ID No. 72)) were expressed in EXPI293™ cells. Proteins were purified using Protein A or Protein G affinity chromatography. The proteins were injected intraperitoneal into C57BL/6 mice and blood samples were taken over the course of 360 hours. Concentration of anti-CD40 in the blood was measured via MSD using immobilized anti-kappa light chain antibody and detecting with anti-human Fc antibody and is shown in FIG. 10. To calculate half-lives, data between 48 and 360 hours was fit to an exponential decay equation. The estimated half-lives are shown in Table 10.

Proteins mimicking the activated drug (without anti-Alb) cleared faster than proteins mimicking the intact Prodrug (with anti-Alb). The greatest difference was seen with the H310A H435A Fc mutations.

TABLE 10
Half-life of intact anti-Alb-anti-CD40 molecules and active
anti-CD40 molecules with different Fc mutations in mice

| | | | Half-life |
|---|---|---|---|
| | | Terminal | with anti- |
| | | half-life | Alb/without |
| Protein | Sequence | (hr) | anti-Alb |
| aCD40 H310A | C2849 (SEQ ID No. 77), | 15 | 2.7 |
| H435A (HAHA) | C00697 (SEQ ID No. 72) | | |
| aAlb:aCD40 | C2849 (SEQ ID No. 77), | 40 | |
| H310A H435A | C2909 (SEQ ID No. 88) | | |
| (aAlb HAHA) | | | |
| aCD40 | C2809 (SEQ ID No. 76), | 36 | 1.4 |
| H310A (HA) | C00697 (SEQ ID No. 72) | | |
| aAlb:aCD40 | C2809 (SEQ ID NO. 76), | 51 | |
| H310A | C2909 (SEQ ID No. 88) | | |
| (aAlb HA) | | | |
| aCD40 | C00696 (SEQ ID No. 75), | 215 | 1.4 |
| (wt) | C00697 (SEQ ID No. 72) | | |
| aAlb:aCD40 | C00696 (SEQ ID No. 75), | 309 | |
| (aAlb wt) | C2909 (SEQ ID No. 88) | | |

Sequence Table
aAlb CD40 masks

| SEQ ID NO: | Description | AA Sequence |
|---|---|---|
| 48 | aAlb-CD-2-C06 | EVQLVESGGGLVQPGNSLTLSCAASGFTESKFGMSWVRQAR |
| | (mask070) | AASAGQGLEWVSSISGSGRDTLYADSVKGRFTISRDNAKTT |
| | | LYLQMNSLRPEDTAVYYCTIGGSLSVSSQGTLVTVSS |
| 49 | aAlb-CD-2-C05 | EVQLVESGGGLVQPGNSLTLSCAASGFTESKFGMSWVRQAL |
| | | GRHESSVGLEWVSSISGSGRDTLYADSVKGRFTISRDNAKT |
| | | TLYLQMNSLRPEDTAVYYCTIGGSLSVSSQGTLVTVSS |
| 50 | aAlb-CD-2-D06 | EVQLVESGGGLVQPGNSLTLSCAASGFTESKFGMSWVRQAM |
| | | NRHENDRGLEWVSSISGSGRDTLYADSVKGRFTISRDNAKT |
| | | TLYLQMNSLRPEDTAVYYCTIGGSLSVSSQGTLVTVSS |
| 51 | aAlb-CD-2-D07 | EVQLVESGGGLVQPGNSLTLSCAASGFTESKFGMSWVRQAR |
| | | AATAGSGLEWVSSISGSGRDTLYADSVKGRFTISRDNAKTT |
| | | LYLQMNSLRPEDTAVYYCTIGGSLSVSSQGTLVTVSS |
| 52 | aAlb-CD-1-F06 | EVQLVESGGGLVQPGNSLTLSCAASGFTESKFGMSWVRQAV |
| | | RTRRTSGGLEWVSSISGSGRDTLYADSVKGRFTISRDNAKT |
| | | TLYLQMNSLRPEDTAVYYCTIGGSLSVSSQGTLVTVSS |
| 53 | aAlb-CD-2-C11 | EVQLVESGGGLVQPGNSLTLSCAASGFTESKFGMSWVRQAL |
| | | GRRETSVGLEWVSSISGSGRDTLYADSVKGRFTISRDNAKT |
| | | TLYLQMNSLRPEDTAVYYCTIGGSLSVSSQGTLVTVSS |
| 54 | aAlb-CD-2-F10 | EVQLVESGGGLVQPGNSLTLSCAASGFTESKFGMSWVRQAV |
| | | GPRETSGGLEWVSSISGSGRDTLYADSVKGRFTISRDNAKT |
| | | TLYLQMNSLRPEDTAVYYCTIGGSLSVSSQGTLVTVSS |
| 55 | aAlb-CD-2-B07 | EVQLVESGGGLVQPGNSLTLSCAASGFTESKFGMSWVRQAV |
| | | RPRGTGGLEWVSSISGSGRDTLYADSVKGRFTISRDNAKTT |
| | | LYLQMNSLRPEDTAVYYCTIGGSLSVSSQGTLVTVSS |
| 56 | aAlb-CD-2-A10 | EVQLVESGGGLVQPGNSLTLSCAASGFTESKFGMSWVRQAV |
| | | GTRETSGGLEWVSSISGSGRDTLYADSVKGRFTISRDNAKT |
| | | TLYLQMNSLRPEDTAVYYCTIGGSLSVSSQGTLVTVSS |
| 57 | aAlb-CD-2-D09 | EVQLVESGGGLVQPGNSLTLSCAASGFTESKFGMSWVRQAV |
| | | RTRGTSGGLEWVSSISGSGRDTLYADSVKGRFTISRDNAKT |
| | | TLYLQMNSLRPEDTAVYYCTIGGSLSVSSQGTLVTVSS |
| 58 | aAlb-CD-1-A09 | EVQLVESGGGLVQPGNSLTLSCAASGFTESKFGMSWVRQAP |
| | | GSQKPGLEWVSSISGSGRDTLYADSVKGRFTISRDNAKTTL |
| | | YLQMNSLRPEDTAVYYCTIGGSLSVSSQGTLVTVSS |
| 59 | aAlb-CD-2-D08 | EVQLVESGGGLVQPGNSLTLSCAASGFTESKFGMSWVRQAT |
| | | SWRHERGLEWVSSISGSGRDTLYADSVKGRFTISRDNAKTT |
| | | LYLQMNSLRPEDTAVYYCTIGGSLSVSSQGTLVTVSS |
| 60 | aAlb-CD-2-F09 | EVQLVESGGGLVQPGNSLTLSCAASGFTESKFGMSWVRQAP |
| | | YSQPTKAGLEWVSSISGSGRDTLYADSVKGRFTISRDNAKT |
| | | TLYLQMNSLRPEDTAVYYCTIGGSLSVSSQGTLVTVSS |
| 61 | Non-cleavable | GGGGSGGGSGGGGSGGGS |
| | linker | |
| 62 | aAlb-wt | EVQLVESGGGLVQPGNSLRLSCAASGFTESKFGMSWVRQAP |
| | | GKGLEWVSSISGSGRDTLYADSVKGRFTISRDNAKTTLYLQ |
| | | MNSLRPEDTAVYYCTIGGSLSVSSQGTLVTVSS |

aAlb CTLA4 masks

| SEQ ID NO: | Description | AA Sequence |
|---|---|---|
| 63 | aAlb-CT-A5 | EVQLVESGGGLVQPGNSLTLSCAASGFTESKFGMSWVRQAP |
| | (mask078) | HYAGLITGLEWVSSISGSGRDTLYADSVKGRFTISRDNAKT |
| | | TLYLQMNSLRPEDTAVYYCTIGGSLSVSSQGTLVTVSS |
| 64 | aAlb-CT-A11 | EVQLVESGGGLVQPGNSLTLSCAASGFTESKFGMSWVRQAE |
| | (mask071) | NLRGLRTGLEWVSSISGSGRDTLYADSVKGRFTISRDNAKT |
| | | TLYLQMNSLRPEDTAVYYCTIGGSLSVSSQGTLVTVSS |
| 65 | aAlb-CT-B9 | EVQLVESGGGLVQPGNSLTLSCAASGFTESKFGMSWVRQAN |
| | | ASSSHGLEWVSSISGSGRDTLYADSVKGRFTISRDNAKTTL |
| | | YLQMNSLRPEDTAVYYCTIGGSLSVSSQGTLVTVSS |
| 66 | aAlb-CT-B3 | EVQLVESGGGLVQPGNSLTLSCAASGFTESKFGMSWVRQAA |
| | | ASADAGLEWVSSISGSGRDTLYADSVKGRFTISRDNAKTTL |
| | | YLQMNSLRPEDTAVYYCTIGGSLSVSSQGTLVTVSS |
| 67 | aAlb-CT-B1 | EVQLVESGGGLVQPGNSLTLSCAASGFTESKFGMSWVRQAA |
| | | WGTSDAGLEWVSSISGSGRDTLYADSVKGRFTISRDNAKTT |
| | | LYLQMNSLRPEDTAVYYCTIGGSLSVSSQGTLVTVSS |
| 68 | aAlb-CT-A10 | EVQLVESGGGLVQPGNSLTLSCAASGFTESKFGMSWVRQAN |
| | | GTSERGLEWVSSISGSGRDTLYADSVKGRFTISRDNAKTTL |
| | | YLQMNSLRPEDTAVYYCTIGGSLSVSSQGTLVTVSS |
| 69 | aAlb-CT-A4 | EVQLVESGGGLVQPGNSLTLSCAASGFTESKFGMSWVRQAA |
| | | APGSTGLEWVSSISGSGRDTLYADSVKGRFTISRDNAKTTL |
| | | YLQMNSLRPEDTAVYYCTIGGSLSVSSQGTLVTVSS |
| 70 | aAlb-CT-A7 | EVQLVESGGGLVQPGNSLTLSCAASGFTESKFGMSWVRQAG |
| | | KDKKDGLEWVSSISGSGRDTLYADSVKGRFTISRDNAKTTL |
| | | YLQMNSLRPEDTAVYYCTIGGSLSVSSQGTLVTVSS |
| 71 | aAlb-CT-D5 | EVQLVESGGGLVQPGNSLTLSCAASGFTESKFGMSWVRQAS |
| | | HGRRQGLEWVSSISGSGRDTLYADSVKGRFTISRDNAKTTL |
| | | YLQMNSLRPEDTAVYYCTIGGSLSVSSQGTLVTVSS |

Antibodies

| SEQ ID NO: | Description | AA Sequence |
|---|---|---|
| 72 | C00697 aCD40 | DIQMTQSPSSVSASVGDRVTITCRASQGIYSWLAWYQQKPG |
| | Light Chain | KAPNLLIYTASTLQSGVPSRFSGSGSGTDFTLTISSLQPED |
| | | FATYYCQQANIFPLTFGGGTKVEIKRTVAAPSVFIFPPSDE |
| | | QLKSGTASVVCLLNNFYPREAKVQWKVDNALQSGNSQESVT |
| | | EQDSKDSTYSLSSTLTLSKADYEKHKVYACEVTHQGLSSPV |
| | | TKSFNRGEC |
| 73 | C00698 aCD40 | QVQLVQSGAEVKKPGASVKVSCKASGYTFTGYYMHWVRQAP |
| | Heavy Chain | GQGLEWMGWINPDSGGTNYAQKFQGRVTMTRDTSISTAYME |
| | IgG1 | LNRLRSDDTAVYYCARDQPLGYCTNGVCSYFDYWGQGTLVT |
| | | VSSASTKGPSVFPLAPSSKSTSGGTAALGCLVKDYFPEPVT |
| | | VSWNSGALTSGVHTFPAVLQSSGLYSLSSVVTVPSSSLGTQ |
| | | TYICNVNHKPSNTKVDKKVEPKSCDKTHTCPPCPAPELLGG |
| | | PSVFLFPPKPKDTLMISRTPEVTCVVVDVSHEDPEVKFNWY |
| | | VDGVEVHNAKTKPREEQYNSTYRVVSVLTVLHQDWLNGKEY |
| | | KCKVSNKALPAPIEKTISKAKGQPREPQVYTLPPSREEMTK |
| | | NQVSLTCLVKGFYPSDIAVEWESNGQPENNYKTTPPVLDSD |
| | | GSFFLYSKLTVDKSRWQQGNVESCSVMHEALHNHYTQKSLS |
| | | LSPGK |
| 74 | C2764 aCD40 | QVQLVQSGAEVKKPGASVKVSCKASGYTFTGYYMHWVRQAP |
| | Heavy Chain | GQGLEWMGWINPDSGGTNYAQKFQGRVTMTRDTSISTAYME |
| | GDSE | LNRLRSDDTAVYYCARDQPLGYCTNGVCSYFDYWGQGTLVT |
| | | VSSASTKGPSVFPLAPSSKSTSGGTAALGCLVKDYFPEPVT |
| | | VSWNSGALTSGVHTFPAVLQSSGLYSLSSVVTVPSSSLGTQ |
| | | TYICNVNHKPSNTKVDKKVEPKSCDKTHTCPPCPAPELLDG |
| | | PSVFLFPPKPKDTLMISRTPEVTCVVVDVEHEDPEVKFNWY |
| | | VDGVEVHNAKTKPREEQYNSTYRVVSVLTVLHQDWLNGKEY |
| | | KCKVSNKALPAPIEKTISKAKGQPREPQVYTLPPSREEMTK |
| | | NQVSLTCLVKGFYPSDIAVEWESNGQPENNYKTTPPVLDSD |
| | | GSFFLYSKLTVDKSRWQQGNVESCSVMHEALHNHYTQKSLS |
| | | LSPGK |
| 75 | C00696 aCD40 | QVQLVQSGAEVKKPGASVKVSCKASGYTFTGYYMHWVRQAP |
| | Heavy Chain | GQGLEWMGWINPDSGGTNYAQKFQGRVTMTRDTSISTAYME |
| | IgG2 | LNRLRSDDTAVYYCARDQPLGYCTNGVCSYFDYWGQGTLVT |
| | | VSSASTKGPSVFPLAPCSRSTSESTAALGCLVKDYFPEPVT |
| | | VSWNSGALTSGVHTFPAVLQSSGLYSLSSVVTVPSSNFGTQ |
| | | TYTCNVDHKPSNTKVDKTVERKCCVECPPCPAPPVAGPSVF |
| | | LFPPKPKDTLMISRTPEVTCVVVDVSHEDPEVQFNWYVDGV |
| | | EVHNAKTKPREEQFNSTFRVVSVLTVVHQDWLNGKEYKCKV |
| | | SNKGLPAPIEKTISKTKGQPREPQVYTLPPSREEMTKNQVS |
| | | LTCLVKGFYPSDIAVEWESNGQPENNYKTTPPMLDSDGSFF |
| | | LYSKLTVDKSRWQQGNVFSCSVMHEALHNHYTQKSLSLSPG |
| | | K |
| 76 | C2809 aCD40 | QVQLVQSGAEVKKPGASVKVSCKASGYTFTGYYMHWVRQAP |
| | Heavy Chain | GQGLEWMGWINPDSGGTNYAQKFQGRVTMTRDTSISTAYME |
| | IgG2 H310A | LNRLRSDDTAVYYCARDQPLGYCTNGVCSYFDYWGQGTLVT |
| | | VSSASTKGPSVFPLAPCSRSTSESTAALGCLVKDYFPEPVT |
| | | VSWNSGALTSGVHTFPAVLQSSGLYSLSSVVTVPSSNFGTQ |
| | | TYTCNVDHKPSNTKVDKTVERKCCVECPPCPAPPVAGPSVF |
| | | LFPPKPKDTLMISRTPEVTCVVVDVSHEDPEVQFNWYVDGV |
| | | EVHNAKTKPREEQFNSTFRVVSVLTVVAQDWLNGKEYKCKV |
| | | SNKGLPAPIEKTISKTKGQPREPQVYTLPPSREEMTKNQVS |
| | | LTCLVKGFYPSDIAVEWESNGQPENNYKTTPPMLDSDGSFF |
| | | LYSKLTVDKSRWQQGNVFSCSVMHEALHNHYTQKSLSLSPG |
| | | K |
| 77 | C2849 aCD40 | QVQLVQSGAEVKKPGASVKVSCKASGYTFTGYYMHWVRQAP |
| | Heavy Chain | GQGLEWMGWINPDSGGTNYAQKFQGRVTMTRDTSISTAYME |
| | IgG2 H310A | LNRLRSDDTAVYYCARDQPLGYCTNGVCSYFDYWGQGTLVT |
| | H435A | VSSASTKGPSVFPLAPCSRSTSESTAALGCLVKDYFPEPVT |
| | | VSWNSGALTSGVHTFPAVLQSSGLYSLSSVVTVPSSNFGTQ |
| | | TYTCNVDHKPSNTKVDKTVERKCCVECPPCPAPPVAGPSVF |
| | | LFPPKPKDTLMISRTPEVTCVVVDVSHEDPEVQFNWYVDGV |
| | | EVHNAKTKPREEQFNSTFRVVSVLTVVAQDWLNGKEYKCKV |
| | | SNKGLPAPIEKTISKTKGQPREPQVYTLPPSREEMTKNQVS |
| | | LTCLVKGFYPSDIAVEWESNGQPENNYKTTPPMLDSDGSFF |
| | | LYSKLTVDKSRWQQGNVFSCSVMHEALHNAYTQKSLSLSPG |
| | | K |
| 78 | C00827 aCTLA4 | EIVLTQSPGTLSLSPGERATLSCRASQSVGSSYLAWYQQKP |
| | Light Chain | GQAPRLLIYGAFSRATGIPDRFSGSGSGTDFTLTISRLEPE |
| | | DFAVYYCQQYGSSPWTFGQGTKVEIKRTVAAPSVFIFPPSD |
| | | EQLKSGTASVVCLLNNFYPREAKVQWKVDNALQSGNSQESV |
| | | TEQDSKDSTYSLSSTLTLSKADYEKHKVYACEVTHQGLSSP |
| | | VTKSFNRGEC |
| 79 | C00826 aCTLA4 | QVQLVESGGGVVQPGRSLRLSCAASGFTFSSYTMHWVRQAP |
| | Heavy Chain | GKGLEWVTFISYDGNNKYYADSVKGRFTISRDNSKNTLYLQ |
| | IgG1 | MNSLRAEDTAIYYCARTGWLGPFDYWGQGTLVTVSSASTKG |
| | | PSVFPLAPSSKSTSGGTAALGCLVKDYFPEPVTVSWNSGAL |
| | | TSGVHTFPAVLQSSGLYSLSSVVTVPSSSLGTQTYICNVNH |
| | | KPSNTKVDKKVEPKSCDKTHTCPPCPAPELLGGPSVFLFPP |
| | | KPKDTLMISRTPEVTCVVVDVSHEDPEVKFNWYVDGVEVHN |
| | | AKTKPREEQYNSTYRVVSVLTVLHQDWLNGKEYKCKVSNKA |
| | | LPAPIEKTISKAKGQPREPQVYTLPPSREEMTKNQVSLTCL |
| | | VKGFYPSDIAVEWESNGQPENNYKTTPPVLDSDGSFFLYSK |
| | | LTVDKSRWQQGNVFSCSVMHEALHNHYTQKSLSLSPGK |

aAlb, aPSMA, and aPD1 masked constructs

| SEQ ID NO: | Description | AA Sequence |
|---|---|---|
| 80 | C2882 aAlb- | EVQLVESGGGLVQPGNSLTLSCAASGFTESKFGMSWVRQAR |
| | CD40-mask070 | AASAGQGLEWVSSISGSGRDTLYADSVKGRFTISRDNAKTT |
| | | LYLQMNSLRPEDTAVYYCTIGGSLSVSSQGTLVTVSSGGGG |
| | | SGGGSGGGGSGGGSDIQMTQSPSSVSASVGDRVTITCRASQ |
| | | GIYSWLAWYQQKPGKAPNLLIYTASTLQSGVPSRFSGSGSG |
| | | TDFTLTISSLQPEDFATYYCQQANIFPLTFGGGTKVEIKRT |
| | | VAAPSVFIFPPSDEQLKSGTASVVCLLNNFYPREAKVQWKV |
| | | DNALQSGNSQESVTEQDSKDSTYSLSSTLTLSKADYEKHKV |
| | | YACEVTHQGLSSPVTKSFNRGEC |
| 81 | C2888 aAlb- | EVQLVESGGGLVQPGNSLTLSCAASGFTESKFGMSWVRQAE |
| | CTLA4-mask071 | NLRGLRTGLEWVSSISGSGRDTLYADSVKGRFTISRDNAKT |
| | | TLYLQMNSLRPEDTAVYYCTIGGSLSVSSQGTLVTVSSGGG |
| | | GSGGGSGGGGSGGGSEIVLTQSPGTLSLSPGERATLSCRAS |
| | | QSVGSSYLAWYQQKPGQAPRLLIYGAFSRATGIPDRFSGSG |
| | | SGTDFTLTISRLEPEDFAVYYCQQYGSSPWTFGQGTKVEIK |
| | | RTVAAPSVFIFPPSDEQLKSGTASVVCLLNNFYPREAKVQW |
| | | KVDNALQSGNSQESVTEQDSKDSTYSLSSTLTLSKADYEKH |
| | | KVYACEVTHQGLSSPVTKSFNRGEC |
| 82 | C3268 aAlb- | EVQLVESGGGLVQPGNSLTLSCAASGFTESKFGMSWVRQAP |
| | CTLA4-mask078 | HYAGLITGLEWVSSISGSGRDTLYADSVKGRFTISRDNAKT |
| | | TLYLQMNSLRPEDTAVYYCTIGGSLSVSSQGTLVTVSSGGG |
| | | GSGGGSGGGGSGGGSEIVLTQSPGTLSLSPGERATLSCRAS |
| | | QSVGSSYLAWYQQKPGQAPRLLIYGAFSRATGIPDRFSGSG |
| | | SGTDFTLTISRLEPEDFAVYYCQQYGSSPWTFGQGTKVEIK |
| | | RTVAAPSVFIFPPSDEQLKSGTASVVCLLNNFYPREAKVQW |
| | | KVDNALQSGNSQESVTEQDSKDSTYSLSSTLTLSKADYEKH |
| | | KVYACEVTHQGLSSPVTKSFNRGEC |
| 83 | C3280 aPSMA- | EVQLVESGGGLVQPGGSLTLSCAASRFMISEYHMHWVRQAE |
| | CTLA4-mask071 | NLRGLRTGLEWVSTINPAGTTDYAESVKGRFTISRDNAKNT |
| | | LYLQMNSLKPEDTAVYYCDSYGYRGQGTQVTVSSGGGGSGG |
| | | GSGGGGSGGGSEIVLTQSPGTLSLSPGERATLSCRASQSVG |
| | | SSYLAWYQQKPGQAPRLLIYGAFSRATGIPDRFSGSGSGTD |
| | | FTLTISRLEPEDFAVYYCQQYGSSPWTFGQGTKVEIKRTVA |
| | | APSVFIFPPSDEQLKSGTASVVCLLNNFYPREAKVQWKVDN |
| | | ALQSGNSQESVTEQDSKDSTYSLSSTLTLSKADYEKHKVYA |
| | | CEVTHQGLSSPVTKSFNRGEC |
| 84 | C3281 aPSMA- | EVQLVESGGGLVQPGGSLTLSCAASRFMISEYHMHWVRQAP |
| | CTLA4-mask078 | HYAGLITGLEWVSTINPAGTTDYAESVKGRFTISRDNAKNT |
| | | LYLQMNSLKPEDTAVYYCDSYGYRGQGTQVTVSSGGGGSGG |
| | | GSGGGGSGGGSEIVLTQSPGTLSLSPGERATLSCRASQSVG |
| | | SSYLAWYQQKPGQAPRLLIYGAFSRATGIPDRFSGSGSGTD |
| | | FTLTISRLEPEDFAVYYCQQYGSSPWTFGQGTKVEIKRTVA |
| | | APSVFIFPPSDEQLKSGTASVVCLLNNFYPREAKVQWKVDN |
| | | ALQSGNSQESVTEQDSKDSTYSLSSTLTLSKADYEKHKVYA |
| | | CEVTHQGLSSPVTKSFNRGEC |
| 85 | C3282 aPD1- | QVQLQESGGGLVQAGGSLRLSCAFSGETFDDFDIGWFRQAP |
| | CTLA4-mask078 | HYAGLITGEREVVSCIASRDGSTYYADSVKGRFTISSDNAK |
| | | NTVYLQMNSLKPEDTAAYHCAAGKYKTYAVTCPLYDHWGQG |
| | | TQVTVSSGGGGSGGGSGGGGSGGGSEIVLTQSPGTLSLSPG |
| | | ERATLSCRASQSVGSSYLAWYQQKPGQAPRLLIYGAFSRAT |
| | | GIPDRFSGSGSGTDFTLTISRLEPEDFAVYYCQQYGSSPWT |
| | | FGQGTKVEIKRTVAAPSVFIFPPSDEQLKSGTASVVCLLNN |
| | | FYPREAKVQWKVDNALQSGNSQESVTEQDSKDSTYSLSSTL |
| | | TLSKADYEKHKVYACEVTHQGLSSPVTKSFNRGEC |
| 86 | C3283 aPSMA- | EVQLVESGGGLVQPGGSLTLSCAASRFMISEYHMHWVRQAR |
| | CD40-mask070 | AASAGQGLEWVSTINPAGTTDYAESVKGRFTISRDNAKNTL |
| | | YLQMNSLKPEDTAVYYCDSYGYRGQGTQVTVSSGGGGSGGG |
| | | SGGGGSGGGSDIQMTQSPSSVSASVGDRVTITCRASQGIYS |
| | | WLAWYQQKPGKAPNLLIYTASTLQSGVPSRFSGSGSGTDFT |
| | | LTISSLQPEDFATYYCQQANIFPLTFGGGTKVEIKRTVAAP |
| | | SVFIFPPSDEQLKSGTASVVCLLNNFYPREAKVQWKVDNAL |
| | | QSGNSQESVTEQDSKDSTYSLSSTLTLSKADYEKHKVYACE |
| | | VTHQGLSSPVTKSFNRGEC |
| 87 | C3284 aPD1- | QVQLQESGGGLVQAGGSLRLSCAFSGFTFDDFDIGWFRQAR |
| | CD40-mask070 | AASAGQGEREVVSCIASRDGSTYYADSVKGRFTISSDNAKN |
| | | TVYLQMNSLKPEDTAAYHCAAGKYKTYAVTCPLYDHWGQGT |
| | | QVTVSSGGGGSGGGSGGGGSGGGSDIQMTQSPSSVSASVGD |
| | | RVTITCRASQGIYSWLAWYQQKPGKAPNLLIYTASTLQSGV |
| | | PSRFSGSGSGTDFTLTISSLQPEDFATYYCQQANIFPLTFG |
| | | GGTKVEIKRTVAAPSVFIFPPSDEQLKSGTASVVCLLNNFY |
| | | PREAKVQWKVDNALQSGNSQESVTEQDSKDSTYSLSSTLTL |
| | | SKADYEKHKVYACEVTHQGLSSPVTKSFNRGEC |
| 88 | C2909 aAlb-CD40 | EVQLVESGGGLVQPGNSLRLSCAASGFTESKFGMSWVRQAP |
| | | GKGLEWVSSISGSGRDTLYADSVKGRFTISRDNAKTTLYLQ |
| | | MNSLRPEDTAVYYCTIGGSLSVSSQGTLVTVSSGGGGSGGG |
| | | SGGGGSGGGSDIQMTQSPSSVSASVGDRVTITCRASQGIYS |
| | | WLAWYQQKPGKAPNLLIYTASTLQSGVPSRFSGSGSGTDFT |
| | | LTISSLQPEDFATYYCQQANIFPLTFGGGTKVEIKRTVAAP |
| | | SVFIFPPSDEQLKSGTASVVCLLNNFYPREAKVQWKVDNAL |
| | | QSGNSQESVTEQDSKDSTYSLSSTLTLSKADYEKHKVYACE |
| | | VTHQGLSSPVTKSFNRGEC |
| 89 | C2910 aAlb- | EVQLVESGGGLVQPGNSLRLSCAASGFTESKFGMSWVRQAP |
| | CTLA4 | GKGLEWVSSISGSGRDTLYADSVKGRFTISRDNAKTTLYLQ |
| | | MNSLRPEDTAVYYCTIGGSLSVSSQGTLVTVSSGGGGSGGG |
| | | SGGGGSGGGSEIVLTQSPGTLSLSPGERATLSCRASQSVGS |
| | | SYLAWYQQKPGQAPRLLIYGAFSRATGIPDRFSGSGSGTDF |
| | | TLTISRLEPEDFAVYYCQQYGSSPWTFGQGTKVEIKRTVAA |
| | | PSVFIFPPSDEQLKSGTASVVCLLNNFYPREAKVQWKVDNA |
| | | LQSGNSQESVTEQDSKDSTYSLSSTLTLSKADYEKHKVYAC |
| | | EVTHQGLSSPVTKSFNRGEC |
| 90 | C2905 aAlb | EVQLVESGGGLVQPGNSLTLSCAASGFTESKFGMSWVRQAR |
| | mask070 L040 | AASAGQGLEWVSSISGSGRDTLYADSVKGRFTISRDNAKTT |
| | CD40 | LYLQMNSLRPEDTAVYYCTIGGSLSVSSQGTLVTVSSGGGG |
| | | PQASTGRSGGGGSDIQMTQSPSSVSASVGDRVTITCRASQG |
| | | IYSWLAWYQQKPGKAPNLLIYTASTLQSGVPSRFSGSGSGT |
| | | DFTLTISSLQPEDFATYYCQQANIFPLTFGGGTKVEIKRTV |
| | | AAPSVFIFPPSDEQLKSGTASVVCLLNNFYPREAKVQWKVD |
| | | NALQSGNSQESVTEQDSKDSTYSLSSTLTLSKADYEKHKVY |
| | | ACEVTHQGLSSPVTKSFNRGEC |
| 91 | C2892 aAlb | EVQLVESGGGLVQPGNSLTLSCAASGFTFSKFGMSWVRQAI |
| | mask071 L040 | QPVHTSPGLEWVSSISGSGRDTLYADSVKGRFTISRDNAKT |
| | CTLA4 | TLYLQMNSLRPEDTAVYYCTIGGSLSVSSQGTLVTVSSGGG |
| | | GPQASTGRSGGGSEIVLTQSPGTLSLSPGERATLSCRASQS |
| | | VGSSYLAWYQQKPGQAPRLLIYGAFSRATGIPDRFSGSGSG |
| | | TDFTLTISRLEPEDFAVYYCQQYGSSPWTFGQGTKVEIKRT |
| | | VAAPSVFIFPPSDEQLKSGTASVVCLLNNFYPREAKVQWKV |
| | | DNALQSGNSQESVTEQDSKDSTYSLSSTLTLSKADYEKHKV |
| | | YACEVTHQGLSSPVTKSFNRGEC |

Ligands used in binding assays

| SEQ ID NO: | Description | AA Sequence |
|---|---|---|
| 92 | C00703 | EPPTACREKQYLINSQCCSLCQPGQKLVSDCTEFTETECLP |
| | huCD40::Fc | CGESEFLDTWNRETHCHQHKYCDPNLGLRVQQKGTSETDTI |
| | | CTCEEGWHCTSEACESCVLHRSCSPGFGVKQIATGVSDTIC |
| | | EPCPVGFFSNVSSAFEKCHPWTSCETKDLVVQQAGTNKTDV |
| | | VCGPQDRLRDYKDDDDKGGGGSDKTHTCPPCPAPELLGGPS |
| | | VFLFPPKPKDTLMISRTPEVTCVVVDVSHEDPEVKFNWYVD |
| | | GVEVHNAKTKPREEQYNSTYRVVSVLTVLHQDWLNGKEYKC |
| | | KVSNKALPAPIEKTISKAKGQPREPQVYTLPPSREEMTKNQ |
| | | VSLTCLVKGFYPSDIAVEWESNGQPENNYKTTPPVLDSDGS |
| | | FFLYSKLTVDKSRWQQGNVFSCSVMHEALHNHYTQKSLSLS |
| | | PGK |
| 93 | C00705 | AMHVAQPAVVLASSRGIASFVCEYASPGKATEVRVTVLRQA |
| | huCTLA4::Fc | DSQVTEVCAATYMMGNELTFLDDSICTGTSSGNQVNLTIQG |
| | | LRAMDTGLYICKVELMYPPPYYLGIGNGTQIYVIDPEPCPD |
| | | SDFDYKDDDDKGGGGSDKTHTCPPCPAPELLGGPSVFLFPP |
| | | KPKDTLMISRTPEVTCVVVDVSHEDPEVKFNWYVDGVEVHN |
| | | AKTKPREEQYNSTYRVVSVLTVLHQDWLNGKEYKCKVSNKA |
| | | LPAPIEKTISKAKGQPREPQVYTLPPSREEMTKNQVSLTCL |
| | | VKGFYPSDIAVEWESNGQPENNYKTTPPVLDSDGSFFLYSK |
| | | LTVDKSRWQQGNVFSCSVMHEALHNHYTQKSLSLSPGK |
| 94 | C00651 | DKTHTCPPCPAPELLGGPSVFLFPPKPKDTLMISRTPEVTC |
| | Fc::huPSMA | VVVDVSHEDPEVKFNWYVDGVEVHNAKTKPREEQYNSTYRV |
| | | VSVLTVLHQDWLNGKEYKCKVSNKALPAPIEKTISKAKGQP |
| | | REPQVYTLPPSREEMTKNQVSLTCLVKGFYPSDIAVEWESN |
| | | GQPENNYKTTPPVLDSDGSFFLYSKLTVDKSRWQQGNVFSC |
| | | SVMHEALHNHYTQKSLSLSPGDYKDDDDKSSNEATNITPKH |
| | | NMKAFLDELKAENIKKFLYNFTQIPHLAGTEQNFQLAKQIQ |
| | | SQWKEFGLDSVELAHYDVLLSYPNKTHPNYISIINEDGNEI |
| | | ENTSLFEPPPPGYENVSDIVPPFSAFSPQGMPEGDLVYVNY |
| | | ARTEDFFKLERDMKINCSGKIVIARYGKVFRGNKVKNAQLA |
| | | GAKGVILYSDPADYFAPGVKSYPDGWNLPGGGVQRGNILNL |
| | | NGAGDPLTPGYPANEYAYRRGIAEAVGLPSIPVHPIGYYDA |
| | | QKLLEKMGGSAPPDSSWRGSLKVPYNVGPGFTGNFSTQKVK |
| | | MHIHSTNEVTRIYNVIGTLRGAVEPDRYVILGGHRDSWVFG |
| | | GIDPQSGAAVVHEIVRSFGTLKKEGWRPRRTILFASWDAEE |
| | | FGLLGSTEWAEENSRLLQERGVAYINADSSIEGNYTLRVDC |
| | | TPLMYSLVHNLTKELKSPDEGFEGKSLYESWTKKSPSPEFS |
| | | GMPRISKLGSGNDFEVFFQRLGIASGRARYTKNWETNKFSG |
| | | YPLYHSVYETYELVEKEYDPMFKYHLTVAQVRGGMVFELAN |
| | | SIVLPFDCRDYAVVLRKYADKIYSISMKHPQEMKTYSVSFD |
| | | SLFSAVKNFTEIASKFSERLQDFDKSNPIVLRMMNDQLMFL |
| | | ERAFIDPLGLPDRPFYRHVIYAPSSHNKYAGESFPGIYDAL |
| | | FDIESKVDPSKAWGEVKRQIYVAAFTVQAAAETLSEVA |

aPSMA CTLA4 mask

| | | |
|---|---|---|
| 95 | aPSMA-mask 071 | EVQLVESGGGLVQPGGSLTLSCAASRFMISEYHMHWVRQAE |
| | | NLRGLRTGLEWVSTINPAGTTDYAESVKGRFTISRDNAKNT |
| | | LYLQMNSLKPEDTAVYYCDSYGYRGQGTQVTVSS |
| 96 | aPSMA-mask 078 | EVQLVESGGGLVQPGGSLTLSCAASRFMISEYHMHWVRQAP |
| | | HYAGLITGLEWVSTINPAGTTDYAESVKGRFTISRDNAKNT |
| | | LYLQMNSLKPEDTAVYYCDSYGYRGQGTQVTVSS |

aPSMA CD40 mask

| | | |
|---|---|---|
| 97 | aPSMA-mask 070 | EVQLVESGGGLVQPGGSLTLSCAASRFMISEYHMHWVRQAR |
| | | AASAGQGLEWVSTINPAGTTDYAESVKGRFTISRDNAKNTL |
| | | YLQMNSLKPEDTAVYYCDSYGYRGQGTQVTVSS |

aPD1 CTLA4 mask

| | | |
|---|---|---|
| 98 | C3282 aPD1- | QVQLQESGGGLVQAGGSLRLSCAFSGFTFDDFDIGWFRQAP |
| | CTLA4-mask078 | HYAGLITGEREVVSCIASRDGSTYYADSVKGRFTISSDNAK |
| | | NTVYLQMNSLKPEDTAAYHCAAGKYKTYAVTCPLYDHWGQG |
| | | TQVTVSS |

aPD1 CD40 mask

| | | |
|---|---|---|
| 99 | C3284 aPD1- | QVQLQESGGGLVQAGGSLRLSCAFSGFTFDDFDIGWFRQAR |
| | CD40-mask070 | AASAGQGEREVVSCIASRDGSTYYADSVKGRFTISSDNAKN |
| | | TVYLQMNSLKPEDTAAYHCAAGKYKTYAVTCPLYDHWGQGT |
| | | QVTVSS |

Linker

| | | |
|---|---|---|
| 100 | L40 linker from | GGGGPQASTGRSGGGGS |
| | C2905 aAlb | |
| | mask070 L040 | |
| | CD40 | |
| 101 | L40 linker from | GGGGPQASTGRSGGGS |
| | C2892 aAlb | |
| | mask071 L040 | |
| | CTLA4 | |
| 102 | L001 linker | GGGGKPLGLQARVVGGGGT |

What is claimed is:

1. A pro immune modulating molecule comprising a first dual binding moiety and a second dual binding moiety and an antibody, wherein each dual binding moiety comprises a cleavable linker, a non-CDR loop, and CDRs for binding a bulk serum protein or a dual binding moiety target, wherein the antibody comprises:
a first light chain polypeptide and a second light chain polypeptide each comprising a light chain variable domain (VL) and a light chain constant domain (CL domain);
a first heavy chain polypeptide and a second heavy chain polypeptide each comprising a variable heavy chain domain (VH), a heavy chain constant region domain (CH1), and constant region domains CH2 and CH3, and
wherein the first dual binding moiety and the second dual binding moiety are capable of masking the binding of the antibody to an antibody target, and wherein upon cleavage of the linkers in the first dual binding moiety and the second dual binding moiety the molecule is activated and the antibody is able to bind the antibody target.

2. The pro immune modulating molecule of claim 1, wherein the first dual binding moiety is connected, via its linker, to the N-terminus of the first light chain polypeptide and the second dual binding moiety is connected, via its linker, to the N-terminus of the second light chain polypeptide.

3. The pro immune modulating molecule of claim 1, wherein the first dual binding moiety is connected, via its linker, to the N-terminus of the first heavy chain polypeptide and the second dual binding moiety is connected, via its linker, to the N-terminus of the second heavy chain polypeptide.

4. The pro immune modulating molecule of any one of claims 1-3, wherein the non-CDR loops of the first dual binding moiety and the second dual binding moiety provide binding sites that enable binding of the first dual binding moiety and the second dual binding moiety to the antibody.

5. The pro immune modulating molecule of any one of claims 1-4, wherein the first dual binding moiety and the second dual binding moiety each comprises at least three CDRs (CDR1, CDR2, CDR3) for binding a bulk serum protein.

6. The pro immune modulating molecule of any one of claims 1-4, wherein the first dual binding moiety and the second dual binding moiety each comprises at least three CDRs (CDR1, CDR2, CDR3) for binding the dual binding moiety target.

7. The pro immune modulating molecule of any one of claims 1-6, wherein the non-CDR loop comprises at least one of: an AB loop, a C″D loop, an EF loop, and a CC′ loop and the binding site specific for the antibody is provided by one or more of the AB loop, the C″D loop, the EF loop, and the CC′ loop.

8. The pro immune modulating molecule of claim 7, wherein the binding site specific for the antibody is provided by the CC′ loop.

9. The pro immune modulating molecule of any one of claims 1-8, wherein the antibody target comprises a tumor antigen, an immune modulatory protein, an immune cell, or a T cell.

10. The pro immune modulating molecule of any one of claims 5 and 7-9, wherein the bulk serum protein is at least one of: albumin, transferrin, IgG1, IgG2, IgG4, IgG3, IgA monomer, Factor XIII, Fibrinogen, IgE, and pentameric IgM, or any combinations thereof.

11. The pro immune modulating molecule of any one of claims 5 and 7-9, wherein the bulk serum protein is human serum albumin (ALB) and the antibody target comprises the tumor antigen, the immune modulatory protein, the immune cell, or the T cell.

12. The pro immune modulating molecule of any one of claims 6-9, wherein the dual binding moiety target comprises a tumor antigen, an immune modulatory protein, an immune cell, or a T cell.

13. The pro immune modulating molecule of any one of claims 6-9 and 12, wherein the antibody target and the dual binding moiety target are different.

14. The pro immune modulating molecule of any one of claims 9-13, wherein the antibody target is the tumor antigen and wherein the tumor antigen is selected from the group consisting of EGFR, PSMA, EpCAM, BCMA, 5T4, AFP, Axl, B7-H3, Cadherin-6, CAIX, CD117, CD123, CD138, CD166, CD19, CD20, CD205, CD22, CD30, CD33, CD352, CD37, CD38, CD44, CD52, CD56, CD70, CD71, CD74, CD79b, CEACAMS, c-MET, DLL3, EphA2, FAP, FGFR2, FGFR3, glypican-3, FLT-3, FOLR1, gpNMB, HER2, HPV-16 E6, HPV-16 E7, ITGA3, SLC39A6, Mesothelin, Muc1, Muc16, NaPi2b, Nectin-4, P-cadherin, Prolactin R, PSCA, PTK7, ROR1, SLC44A4, SLTRKS, SLTRK6, STEAP1, TIM1, Trop2, and WT1.

15. The pro immune modulating molecule of any one of claims 12-14, wherein the dual binding moiety target is the tumor antigen and wherein the tumor antigen is selected from the group consisting of EGFR, PSMA, EpCAM, BCMA, 5T4, AFP, Axl, B7-H3, Cadherin-6, CAIX, CD117, CD123, CD138, CD166, CD19, CD20, CD205, CD22, CD30, CD33, CD352, CD37, CD38, CD44, CD52, CD56, CD70, CD71, CD74, CD79b, CEACAM5, c-MET, DLL3, EphA2, FAP, FGFR2, FGFR3, glypican-3, FLT-3, FOLR1, gpNMB, HER2, HPV-16 E6, HPV-16 E7, ITGA3, SLC39A6, Mesothelin, Muc1, Muc16, NaPi2b, Nectin-4, P-cadherin, Prolactin R, PSCA, PTK7, ROR1, SLC44A4, SLTRK5, SLTRK6, STEAP1, TIM1, Trop2, and WT1.

16. The pro immune modulating molecule of any one of claims 9-13, wherein the antibody target is the immune modulatory protein.

17. The pro immune modulating molecule of claim 16, wherein the immune modulatory protein is selected from the group consisting of CTLA-4, CD27, CD137, 2B4, TIGIT, CD155, ICOS, HVEM, CD40L, LIGHT, TIM-1, OX40, DNAM-1, PD-L1, PD1, PD-L2, CD8, CD40, CEACAM1, CD48, CD70, A2AR, CD39, CD73, B7-H3, B7-H4, BTLA, IDO1, IDO2, TDO, KIR, LAG-3, TIM-3, VISTA, IL6-R, IL-6, TNFα, CD19, CD20, CD22, CD52, integrin a4, integrin a4b7, CD11a, CTLA4-Ig fusion, IL-17, IL12/23, IL12, IL23, and TGF-beta.

18. The pro immune modulating molecule of any one of claims 12-14 and 16-17, wherein the dual binding moiety target is the immune modulatory protein.

19. The pro immune modulating molecule of claim 18, wherein the immune modulatory protein is selected from the group consisting of CTLA-4, CD27, CD137, 2B4, TIGIT, CD155, ICOS, HVEM, CD40L, LIGHT, TIM-1, OX40, DNAM-1, PD-L1, PD1, PD-L2, CD8, CD40, CEACAM1, CD48, CD70, A2AR, CD39, CD73, B7-H3, B7-H4, BTLA, IDO1, IDO2, TDO, KIR, LAG-3, TIM-3, VISTA, IL6-R, IL-6, TNFα, CD19, CD20, CD22, CD52, integrin a4, integrin a4b7, CD11a, CTLA4-Ig fusion, IL-17, IL12/23, IL12, IL23, and TGF-beta.

20. The pro immune modulating molecule of any one of claims 9 and 12-13, wherein the antibody target and the dual binding moiety target are independently the immune cell.

21. The pro immune modulating molecule of any one of claims 9 and 12-13, wherein the antibody target and the dual binding moiety target are independently the T cell.

22. The pro immune modulating molecule any one of claims 9 and 12-13, wherein the antibody target and the dual binding moiety target are independently CD3.

23. The pro immune modulating molecule of any one of claims 1-5, 7-11, 14, 16-17, and 20-22, wherein the first dual binding moiety and the second dual binding moiety independently comprises a sequence selected from the group consisting of SEQ ID Nos. 48-60 and 63-71.

24. The pro immune modulating molecule of any one of claims 14-22, wherein the antibody target is CTLA-4 and the dual binding moiety target is PSMA.

25. The pro immune modulating molecule of claim 24, wherein the light chain polypeptides of the antibody comprise the sequence of SEQ ID No. 78 and the heavy chain polypeptides of the antibody comprise the sequence of SEQ ID No. 79, and wherein the first dual binding moiety and the second dual binding moiety each comprises the sequence of SEQ ID No. 95 or SEQ ID No. 96.

26. The pro immune modulating molecule of any one of claim 14-22, wherein the antibody target is CD40 and the dual binding moiety target is PSMA.

27. The pro immune modulating molecule of claim 26, wherein the light chain polypeptides of the antibody comprise the sequence of SEQ ID No. 72 and the heavy chain polypeptides of the antibody comprise the sequence of SEQ ID No. 73, SEQ ID No. 74, SEQ ID No. 75, SEQ ID No. 76, or SEQ ID No. 77, and wherein the first dual binding moiety and the second dual binding moiety each comprises the sequence of SEQ ID No. 97.

28. The pro immune modulating molecule of any one of claims 14-22, wherein the first target is CD40 and the second target is PD1.

29. The pro immune modulating molecule of claim 28, wherein the light chain polypeptides of the antibody comprise the sequence of SEQ ID No. 72 and the heavy chain polypeptides of the antibody comprise the sequence of SEQ ID No. 73, SEQ ID No. 74, SEQ ID No. 75, SEQ ID No. 76, or SEQ ID No. 77, and wherein the first dual binding moiety and the second dual binding moiety each comprises the sequence of SEQ ID No. 99.

30. The pro immune modulating molecule of any one of claims 14-22, wherein the antibody target is CTLA-4 and the dual binding moiety target is PD1.

31. The pro immune modulating molecule of claim 30, wherein the light chain polypeptides of the antibody comprise the sequence of SEQ ID No. 78 and the heavy chain polypeptides of the antibody comprise the sequence of SEQ ID No. 79, and wherein the first dual binding moiety and the second dual binding moiety each comprises the sequence of SEQ ID No. 98.

32. The pro immune modulating molecule of any one of claims 1-31, wherein the cleavable linker of each dual binding moiety comprises the sequence of SEQ ID No. 100, 101, or 102.

33. The pro immune modulating molecule of any one of claims 14, 16-17, and 20-23, wherein the antibody target is CTLA4 or CD40 and the first dual binding moiety and the second dual binding moiety each comprises CDRs for binding the bulk serum protein.

34. The pro immune modulating molecule of claim 33, wherein the antibody target is CTLA4 and the light chain polypeptides of the antibody comprise the sequence of SEQ ID No. 78 and the heavy chain polypeptides of the antibody comprise the sequence of SEQ ID No. 79, and the first and the second dual binding moieties each comprises a sequence selected from SEQ ID Nos. 63-71.

35. The pro immune modulating molecule of claim 33, wherein the antibody target is CD40 the light chain polypeptides of the antibody comprise the sequence of SEQ ID No. 72 and the heavy chain polypeptides of the antibody comprise the sequence of SEQ ID No. 73, SEQ ID No. 74, SEQ ID No. 75, SEQ ID No. 76, or SEQ ID No. 77, and the first and the second dual binding moieties each comprises a sequence selected from SEQ ID Nos. 48-60.

36. The pro immune modulating molecule of any one of claims 1-5, 7-9, 14, 16-17, 20-23 and 32-35, comprising at least one of:
(a) the CH2 domain of at least one of the first heavy chain polypeptide and the second heavy chain polypeptide comprises one or more mutations that reduce FcRn binding; and
(b) the CH3 domain of at least one of the first heavy chain polypeptide and the second heavy chain polypeptide comprises one or more mutations that reduce FcRn binding,
wherein upon cleavage of the linkers of the first dual binding moiety and the second dual binding moiety the molecule is activated and the antibody is able to bind the antibody target, and wherein the activated molecule has a shorter half-life compared to an otherwise identical activated molecule that does not have (a) or (b).

37. The pro immune modulating molecule of any one of claims 1-5, 7-9, 14, 16-17, 20-23 and 32-35, wherein at least one of the first heavy chain polypeptide and the second heavy chain polypeptide comprises a 310A mutation at an amino acid position corresponding to H310 in human IgG1 or IgG2.

38. The pro immune modulating molecule of any one of claims 1-5, 7-9, 14, 16-17, 20-23 and 32-35, wherein at least one of the first heavy chain polypeptide and the second heavy chain polypeptide comprises a 310A mutation at an amino acid position corresponding to H310 in human IgG1 or IgG2 and a 435A mutation at an amino acid position corresponding to H435 in human IgG1 or IgG2.

39. The pro immune modulating molecule of any one of claims 1-38, wherein the cleavable linker of the first dual binding moiety and the second dual binding moiety each comprises a protease cleavage site.

40. The pro immune modulating molecule of claim 39, wherein the protease cleavage site is recognized by one of a serine protease, a cysteine protease, an aspartate protease, a threonine protease, a glutamic acid protease, a metalloproteinase, a gelatinase, and a asparagine peptide lyase.

41. The pro immune modulating molecule of claim 39 or 40, wherein the protease cleavage site is recognized by one of a Cathepsin B, a Cathepsin C, a Cathepsin D, a Cathepsin E, a Cathepsin K, a Cathepsin L, a kallikrein, a hK1, a hK10, a hK15, a plasmin, a collagenase, a Type IV collagenase, a stromelysin, a Factor Xa, a chymotrypsin-like protease, a trypsin-like protease, a elastase-like protease, a subtilisin-like protease, an actinidain, a bromelain, a calpain, a caspase, a caspase-3, a Mir1-CP, a papain, a HIV-1 protease, a HSV protease, a CMV protease, a chymosin, a renin, a pepsin, a matriptase, a legumain, a plasmepsin, a nepenthesin, a metalloexopeptidase, a metalloendopeptidase, a matrix metalloprotease (MMP), a MMP1, a MMP2, a MMP3, a MMPI, a MMP8, a MMP9, a MMP10, a MMP11, a MMP12, a MMP13, a MMP14, an ADAM10, an ADAM12, an urokinase plasminogen activator (uPA), an enterokinase, a prostate-specific target (PSA, hK3), an interleukin-1β converting enzyme, a thrombin, a FAP (FAP-α), a type II transmembrane serine protease (TTSP), a neutrophil elastase, a cathepsin G, a proteinase 3, a neutrophil serine protease 4, a mast cell chymase, a mast cell tryptase, a dipeptidyl peptidase, and a dipeptidyl peptidase IV (DPPIV/CD26).

42. The pro immune modulating molecule of any one of claims 1-41, wherein the cleavable linker of the first dual binding moiety and the second dual binding moiety each independently comprises the amino acid sequence of SEQ ID No. 100 or SEQ ID No. 101.

43. A pro immune modulating molecule comprising:
an antibody or an antigen binding fragment thereof that is capable of binding an antibody target; and
at least one dual binding moiety that is capable of binding a dual binding moiety target, wherein the at least one dual binding moiety comprises a cleavable linker and a non-CDR loop;
wherein the at least one dual binding moiety is capable of masking the antibody or an antigen binding fragment thereof from binding the antibody target, and wherein upon cleavage of the linker the molecule is activated and the antibody or an antigen binding fragment thereof is able to bind the antibody target .

44. The pro immune modulating molecule of claim 43, wherein the non-CDR loop of the at least one dual binding moiety provides a binding site that enables binding of the dual binding moiety to the antibody or an antigen binding fragment thereof.

45. The pro immune modulating molecule of claim 43 or 44, wherein the at least one dual binding moiety comprises a binding site specific for the dual binding moiety target and wherein the binding site is provided by one or more CDRs.

46. The pro immune modulating molecule of any one of claims 43-45, wherein the antibody target and the dual binding moiety target independently comprises a tumor antigen, an immune modulatory protein, an immune cell, or a T cell.

47. The pro immune modulating molecule of any one of claims 43-46, wherein the antibody target and the dual binding moiety target are different.

48. The pro immune modulating molecule of claim 46 or 47, wherein the antibody target and the dual binding moiety target independently are the tumor antigen and wherein the tumor antigen is selected from the group consisting of EGFR, PSMA, EpCAM, BCMA, 5T4, AFP, Axl, B7-H3, Cadherin-6, CAIX, CD117, CD123, CD138, CD166, CD19, CD20, CD205, CD22, CD30, CD33, CD352, CD37, CD38, CD44, CD52, CD56, CD70, CD71, CD74, CD79b, CEACAMS, c-MET, DLL3, EphA2, FAP, FGFR2, FGFR3, glypican-3, FLT-3, FOLR1, gpNMB, HER2, HPV-16 E6, HPV-16 E7, ITGA3, SLC39A6, Mesothelin, Muc1, Muc16, NaPi2b, Nectin-4, P-cadherin, Prolactin R, PSCA, PTK7, ROR1, SLC44A4, SLTRKS, SLTRK6, STEAP1, TIM1, Trop2, and WT1.

49. The pro immune modulating molecule of claim 46 or 47, wherein the antibody target and the dual binding moiety target independently are the immune modulatory protein wherein the immune modulatory protein is selected from the group consisting of CTLA-4, CD27, CD137, 2B4, TIGIT, CD155, ICOS, HVEM, CD40L, LIGHT, TIM-1, OX40, DNAM-1, PD-L1, PD1, PD-L2, CD8, CD40, CEACAM1, CD48, CD70, A2AR, CD39, CD73, B7-H3, B7-H4, BTLA, IDO1, IDO2, TDO, KIR, LAG-3, TIM-3, VISTA, IL6-R, IL-6, TNFα, CD19, CD20, CD22, CD52, integrin a4, integrin a4b7, CD11a, CTLA4-Ig fusion, IL-17, IL12/23, IL12, IL23, and TGF-beta.

50. The pro immune modulating molecule of claim 46 or 47, wherein the antibody target and the dual binding moiety target independently are the immune cell.

51. The pro immune modulating molecule of any one of claim 46 or 47, wherein the antibody target and the dual binding moiety target independently are the T cell.

52. The pro immune modulating molecule of claim 46 or 47, wherein the antibody target and the dual binding moiety target independently are the CD3.

53. The pro immune modulating molecule of any one of claims 48-52, wherein the antibody target is CTLA-4 and the dual binding moiety target is PSMA.

54. The pro immune modulating molecule of claim 53, wherein the dual binding moiety comprises the sequence of SEQ ID No. 95 or SEQ ID No. 96.

55. The pro immune modulating molecule of any one of claim 48-52, wherein the antibody target is CD40 and the dual binding moiety target is PSMA.

56. The pro immune modulating molecule of claim 55, wherein the dual binding moiety comprises the sequence of SEQ ID No. 97.

57. The pro immune modulating molecule of any one of claims 48-52, wherein the antibody target is CD40 and the dual binding moiety target is PD1.

58. The pro immune modulating molecule of claim 57, wherein the dual binding moiety comprises the sequence of SEQ ID No. 99.

59. The pro immune modulating molecule of any one of claims 48-52, wherein the antibody target is CTLA-4 and the dual binding moiety target is PD1.

60. The pro immune modulating molecule of claim 59, wherein the dual binding moiety comprises the sequence of SEQ ID No. 98.

61. The pro immune modulating molecule of any one of claims 43-60, wherein the cleavable linker of the at least one dual binding moiety comprises a protease cleavage site.

62. The pro immune modulating molecule of claim 61, wherein the protease cleavage site is recognized by one of a serine protease, a cysteine protease, an aspartate protease, a threonine protease, a glutamic acid protease, a metalloproteinase, a gelatinase, and a asparagine peptide lyase.

63. The pro immune modulating molecule of claim 61 or 62, wherein the protease cleavage site is recognized by one of a Cathepsin B, a Cathepsin C, a Cathepsin D, a Cathepsin E, a Cathepsin K, a Cathepsin L, a kallikrein, a hK1, a hK10, a hK15, a plasmin, a collagenase, a Type IV collagenase, a stromelysin, a Factor Xa, a chymotrypsin-like protease, a trypsin-like protease, a elastase-like protease, a subtilisin-like protease, an actinidain, a bromelain, a calpain, a caspase, a caspase-3, a Mir1-CP, a papain, a HIV-1 protease, a HSV protease, a CMV protease, a chymosin, a renin, a pepsin, a matriptase, a legumain, a plasmepsin, a nepenthesin, a metalloexopeptidase, a metalloendopeptidase, a matrix metalloprotease (MMP), a MMP1, a MMP2, a MMP3, a MMPI, a MMP8, a MMP9, a MMP10, a MMP11, a MMP12, a MMP13, a MMP14, an ADAM10, an ADAM12, an urokinase plasminogen activator (uPA), an enterokinase, a prostate-specific target (PSA, hK3), an interleukin-1β converting enzyme, a thrombin, a FAP (FAP-α), a type II transmembrane serine protease (TTSP), a neutrophil elastase, a cathepsin G, a proteinase 3, a neutrophil serine protease 4, a mast cell chymase, a mast cell tryptase, a dipeptidyl peptidase, and a dipeptidyl peptidase IV (DPPIV/CD26).

64. The pro immune modulating molecule of any one of claims 43-63, wherein the cleavable linker of the at least one dual binding moiety comprises the amino acid sequence of SEQ ID No. 100 or SEQ ID No. 101.

65. A pro immune modulating molecule comprising a sequence selected from the group consisting of SEQ ID Nos. 80-91.

66. A pro immune modulating molecule comprising an antibody that is masked from binding its target by a first dual binding moiety and a second dual binding moiety, wherein each dual binding moiety comprises a cleavable linker and a non-CDR loop, wherein the first dual binding moiety is connected to the N-terminus of a first light chain polypeptide of the antibody, via the linker of the first dual binding moiety and the second dual binding moiety is connected to the N-terminus of a second light chain polypeptide of the antibody, via the linker of the second dual binding moiety, and wherein the Fc region of the antibody comprises at least one of:
(a) a 310A mutation at an amino acid position corresponding to H310 in human IgG1 or IgG2;
(b) and a 435A mutation at an amino acid position corresponding to H435 in human IgG1 or IgG2.

67. The pro immune modulating molecule of claim 66, wherein upon cleavage of the linkers in the first dual binding moiety and the second dual binding moiety the molecule is activated and the antibody is able to bind its target, and wherein the activated molecule has a shorter half-life compared to an otherwise identical activated molecule that does not comprise (a) or (b).

68. A method of treating a disease comprising administering to a subject an effective amount of a pro immune modulating molecule according to any one of claims 1-67.

69. The method of claim 68, wherein the subject is a human.

70. The method of claim 68 or 69, wherein the disease is a tumorous disease or an inflammatory disease.


SEQUENCE LISTING (113 sequences)

SEQ ID NO: 1 - protein, 8 aa, Unknown
  Description of Unknown Protease cleavage site sequence
KRALGLPG

SEQ ID NO: 2 - protein, 40 aa, Unknown
  Description of Unknown Protease cleavage site sequence
DEDEDEDEDEDEDEDERPLALWRSDRDRDRDRDRDRDRDR

SEQ ID NO: 3 - protein, 5 aa, Unknown
  Description of Unknown Protease cleavage site sequence
  MOD_RES (3)..(3): Ser or Thr
  MOD_RES (4)..(4): Leu or Ile
  MOD_RES (5)..(5): Ser or Thr
PRXXX

SEQ ID NO: 4 - protein, 5 aa, Unknown
  Description of Unknown Protease cleavage site sequence
LEATA

SEQ ID NO: 5 - protein, 10 aa, Unknown
  Description of Unknown Protease cleavage site sequence
GGAANLVRGG

SEQ ID NO: 6 - protein, 10 aa, Unknown
  Description of Unknown Protease cleavage site sequence
SGRIGFLRTA

SEQ ID NO: 7 - protein, 6 aa, Unknown
  Description of Unknown Protease cleavage site sequence
PLGLAG

SEQ ID NO: 8 - protein, 6 aa, Unknown
  Description of Unknown Protease cleavage site sequence
  MOD_RES (6)..(6): Any amino acid
PLGLAX

SEQ ID NO: 9 - protein, 6 aa, Unknown
  Description of Unknown Protease cleavage site sequence
PLGCAG

SEQ ID NO: 10 - protein, 8 aa, Unknown
  Description of Unknown Protease cleavage site sequence
ESPAYYTA

SEQ ID NO: 11 - protein, 6 aa, Unknown
  Description of Unknown Protease cleavage site sequence
RLQLKL

SEQ ID NO: 12 - protein, 7 aa, Unknown
  Description of Unknown Protease cleavage site sequence
RLQLKAC

SEQ ID NO: 13 - protein, 7 aa, Unknown
  Description of Unknown Protease cleavage site sequence
  MOD_RES (3)..(3): Cit
  MOD_RES (5)..(5): Hof
EPXGXYL

SEQ ID NO: 14 - protein, 5 aa, Unknown
  Description of Unknown Protease cleavage site sequence
SGRSA

SEQ ID NO: 15 - protein, 4 aa, Unknown
  Description of Unknown Protease cleavage site sequence
DAFK

SEQ ID NO: 16 - protein, 5 aa, Unknown
  Description of Unknown Protease cleavage site sequence
GGGRR

SEQ ID NO: 17 - protein, 4 aa, Unknown
  Description of Unknown Protease cleavage site sequence
GFLG

SEQ ID NO: 18 - protein, 4 aa, Unknown
  Description of Unknown Protease cleavage site sequence
ALAL

SEQ ID NO: 19 - protein, 2 aa, Unknown
  Description of Unknown Protease cleavage site sequence
FK

SEQ ID NO: 20 - protein, 3 aa, Unknown
  Description of Unknown Protease cleavage site sequence
NLL

SEQ ID NO: 21 - protein, 5 aa, Unknown
  Description of Unknown Protease cleavage site sequence
PICFF

SEQ ID NO: 22 - protein, 8 aa, Unknown
  Description of Unknown Protease cleavage site sequence
GGPRGLPG

SEQ ID NO: 23 - protein, 6 aa, Unknown
  Description of Unknown Protease cleavage site sequence
HSSKLQ

SEQ ID NO: 24 - protein, 7 aa, Unknown
  Description of Unknown Protease cleavage site sequence
HSSKLQL

SEQ ID NO: 25 - protein, 9 aa, Unknown
  Description of Unknown Protease cleavage site sequence
HSSKLQEDA

SEQ ID NO: 26 - protein, 10 aa, Unknown
  Description of Unknown Protease cleavage site sequence
LVLASSSFGY

SEQ ID NO: 27 - protein, 10 aa, Unknown
  Description of Unknown Protease cleavage site sequence
GVSQNYPIVG

SEQ ID NO: 28 - protein, 10 aa, Unknown
  Description of Unknown Protease cleavage site sequence
GVVQASCRLA

SEQ ID NO: 29 - protein, 4 aa, Unknown
  Description of Unknown Protease cleavage site sequence
  MOD_RES (2)..(2): Pip
FXRS

SEQ ID NO: 30 - protein, 6 aa, Unknown
  Description of Unknown Protease cleavage site sequence
DPRSFL

SEQ ID NO: 31 - protein, 6 aa, Unknown
  Description of Unknown Protease cleavage site sequence
PPRSFL

SEQ ID NO: 32 - protein, 4 aa, Unknown
  Description of Unknown Protease cleavage site sequence
DEVD

SEQ ID NO: 33 - protein, 5 aa, Unknown
  Description of Unknown Protease cleavage site sequence
DEVDP

SEQ ID NO: 34 - protein, 8 aa, Unknown
  Description of Unknown Protease cleavage site sequence
KGSGDVEG

SEQ ID NO: 35 - protein, 6 aa, Unknown
  Description of Unknown Protease cleavage site sequence
GWEHDG

SEQ ID NO: 36 - protein, 7 aa, Unknown
  Description of Unknown Protease cleavage site sequence
EDDDDKA

SEQ ID NO: 37 - protein, 9 aa, Unknown
  Description of Unknown Protease cleavage site sequence
KQEQNPGST

SEQ ID NO: 38 - protein, 6 aa, Unknown
  Description of Unknown Protease cleavage site sequence
GKAFRR

SEQ ID NO: 39 - protein, 4 aa, Unknown
  Description of Unknown Protease cleavage site sequence
DAFK

SEQ ID NO: 40 - protein, 4 aa, Unknown
  Description of Unknown Protease cleavage site sequence
DVLK

SEQ ID NO: 41 - protein, 4 aa, Unknown
  Description of Unknown Protease cleavage site sequence
DAFK

SEQ ID NO: 42 - protein, 7 aa, Unknown
  Description of Unknown Protease cleavage site sequence
ALLLALL

SEQ ID NO: 43 - protein, 6 aa, Artificial Sequence
  Description of Artificial Sequence Synthetic peptide
PLGLWA

SEQ ID NO: 44 - protein, 6 aa, Artificial Sequence
  Description of Artificial Sequence Synthetic peptide
RVLAEA

SEQ ID NO: 45 - protein, 10 aa, Artificial Sequence
  Description of Artificial Sequence Synthetic peptide
EDVVCCSMSY

SEQ ID NO: 46 - protein, 8 aa, Artificial Sequence
  Description of Artificial Sequence Synthetic peptide
GGIEGRGS

SEQ ID NO: 47 - protein, 4 aa, Artificial Sequence
  Description of Artificial Sequence Synthetic peptide
GFLG

SEQ ID NO: 48 - protein, 119 aa, Artificial Sequence
  Description of Artificial Sequence Synthetic polypeptide
EVQLVESGGGLVQPGNSLTLSCAASGFTFSKFGMSWVRQARAASAGQGLEWVSSISGSGRDTLYADSVKGRFTISRDNAKTTLYLQMNSLRPEDTAVYYCTIGGSLSVSSQGTLVTVSS

SEQ ID NO: 49 - protein, 120 aa, Artificial Sequence
  Description of Artificial Sequence Synthetic polypeptide
EVQLVESGGGLVQPGNSLTLSCAASGFTFSKFGMSWVRQALGRHESSVGLEWVSSISGSGRDTLYADSVKGRFTISRDNAKTTLYLQMNSLRPEDTAVYYCTIGGSLSVSSQGTLVTVSS

SEQ ID NO: 50 - protein, 120 aa, Artificial Sequence
  Description of Artificial Sequence Synthetic polypeptide
EVQLVESGGGLVQPGNSLTLSCAASGFTFSKFGMSWVRQAMNRHENDRGLEWVSSISGSGRDTLYADSVKGRFTISRDNAKTTLYLQMNSLRPEDTAVYYCTIGGSLSVSSQGTLVTVSS

SEQ ID NO: 51 - protein, 119 aa, Artificial Sequence
  Description of Artificial Sequence Synthetic polypeptide
EVQLVESGGGLVQPGNSLTLSCAASGFTFSKFGMSWVRQARAATAGSGLEWVSSISGSGRDTLYADSVKGRFTISRDNAKTTLYLQMNSLRPEDTAVYYCTIGGSLSVSSQGTLVTVSS

SEQ ID NO: 52 - protein, 120 aa, Artificial Sequence
  Description of Artificial Sequence Synthetic polypeptide
EVQLVESGGGLVQPGNSLTLSCAASGFTFSKFGMSWVRQAVRTRRTSGGLEWVSSISGSGRDTLYADSVKGRFTISRDNAKTTLYLQMNSLRPEDTAVYYCTIGGSLSVSSQGTLVTVSS

SEQ ID NO: 53 - protein, 120 aa, Artificial Sequence
  Description of Artificial Sequence Synthetic polypeptide
EVQLVESGGGLVQPGNSLTLSCAASGFTFSKFGMSWVRQALGRRETSVGLEWVSSISGSGRDTLYADSVKGRFTISRDNAKTTLYLQMNSLRPEDTAVYYCTIGGSLSVSSQGTLVTVSS

SEQ ID NO: 54 - protein, 120 aa, Artificial Sequence
  Description of Artificial Sequence Synthetic polypeptide
EVQLVESGGGLVQPGNSLTLSCAASGFTFSKFGMSWVRQAVGPRETSGGLEWVSSISGSGRDTLYADSVKGRFTISRDNAKTTLYLQMNSLRPEDTAVYYCTIGGSLSVSSQGTLVTVSS

SEQ ID NO: 55 - protein, 119 aa, Artificial Sequence
  Description of Artificial Sequence Synthetic polypeptide
EVQLVESGGGLVQPGNSLTLSCAASGFTFSKFGMSWVRQAVRPRGTGGLEWVSSISGSGRDTLYADSVKGRFTISRDNAKTTLYLQMNSLRPEDTAVYYCTIGGSLSVSSQGTLVTVSS

SEQ ID NO: 56 - protein, 120 aa, Artificial Sequence
  Description of Artificial Sequence Synthetic polypeptide
EVQLVESGGGLVQPGNSLTLSCAASGFTFSKFGMSWVRQAVGTRETSGGLEWVSSISGSGRDTLYADSVKGRFTISRDNAKTTLYLQMNSLRPEDTAVYYCTIGGSLSVSSQGTLVTVSS

SEQ ID NO: 57 - protein, 120 aa, Artificial Sequence
  Description of Artificial Sequence Synthetic polypeptide
EVQLVESGGGLVQPGNSLTLSCAASGFTFSKFGMSWVRQAVRTRGTSGGLEWVSSISGSGRDTLYADSVKGRFTISRDNAKTTLYLQMNSLRPEDTAVYYCTIGGSLSVSSQGTLVTVSS

SEQ ID NO: 58 - protein, 118 aa, Artificial Sequence
  Description of Artificial Sequence Synthetic polypeptide
EVQLVESGGGLVQPGNSLTLSCAASGFTFSKFGMSWVRQAPGSQKPGLEWVSSISGSGRDTLYADSVKGRFTISRDNAKTTLYLQMNSLRPEDTAVYYCTIGGSLSVSSQGTLVTVSS

SEQ ID NO: 59 - protein, 119 aa, Artificial Sequence
  Description of Artificial Sequence Synthetic polypeptide
EVQLVESGGGLVQPGNSLTLSCAASGFTFSKFGMSWVRQATSWRHERGLEWVSSISGSGRDTLYADSVKGRFTISRDNAKTTLYLQMNSLRPEDTAVYYCTIGGSLSVSSQGTLVTVSS

SEQ ID NO: 60 - protein, 120 aa, Artificial Sequence
  Description of Artificial Sequence Synthetic polypeptide
EVQLVESGGGLVQPGNSLTLSCAASGFTFSKFGMSWVRQAPYSQPTKAGLEWVSSISGSGRDTLYADSVKGRFTISRDNAKTTLYLQMNSLRPEDTAVYYCTIGGSLSVSSQGTLVTVSS

SEQ ID NO: 61 - protein, 18 aa, Artificial Sequence
  Description of Artificial Sequence Synthetic peptide
GGGGSGGGSGGGGSGGGS

SEQ ID NO: 62 - protein, 115 aa, Artificial Sequence
  Description of Artificial Sequence Synthetic polypeptide
EVQLVESGGGLVQPGNSLRLSCAASGFTFSKFGMSWVRQAPGKGLEWVSSISGSGRDTLYADSVKGRFTISRDNAKTTLYLQMNSLRPEDTAVYYCTIGGSLSVSSQGTLVTVSS

SEQ ID NO: 63 - protein, 120 aa, Artificial Sequence
  Description of Artificial Sequence Synthetic polypeptide
EVQLVESGGGLVQPGNSLTLSCAASGFTFSKFGMSWVRQAPHYAGLITGLEWVSSISGSGRDTLYADSVKGRFTISRDNAKTTLYLQMNSLRPEDTAVYYCTIGGSLSVSSQGTLVTVSS

SEQ ID NO: 64 - protein, 120 aa, Artificial Sequence
  Description of Artificial Sequence Synthetic polypeptide
EVQLVESGGGLVQPGNSLTLSCAASGFTFSKFGMSWVRQAENLRGLRTGLEWVSSISGSGRDTLYADSVKGRFTISRDNAKTTLYLQMNSLRPEDTAVYYCTIGGSLSVSSQGTLVTVSS

SEQ ID NO: 65 - protein, 118 aa, Artificial Sequence
  Description of Artificial Sequence Synthetic polypeptide
EVQLVESGGGLVQPGNSLTLSCAASGFTFSKFGMSWVRQANASSSHGLEWVSSISGSGRDTLYADSVKGRFTISRDNAKTTLYLQMNSLRPEDTAVYYCTIGGSLSVSSQGTLVTVSS

SEQ ID NO: 66 - protein, 118 aa, Artificial Sequence
  Description of Artificial Sequence Synthetic polypeptide
EVQLVESGGGLVQPGNSLTLSCAASGFTFSKFGMSWVRQAAASADAGLEWVSSISGSGRDTLYADSVKGRFTISRDNAKTTLYLQMNSLRPEDTAVYYCTIGGSLSVSSQGTLVTVSS

SEQ ID NO: 67 - protein, 119 aa, Artificial Sequence
  Description of Artificial Sequence Synthetic polypeptide
EVQLVESGGGLVQPGNSLTLSCAASGFTFSKFGMSWVRQAAWGTSDAGLEWVSSISGSGRDTLYADSVKGRFTISRDNAKTTLYLQMNSLRPEDTAVYYCTIGGSLSVSSQGTLVTVSS

SEQ ID NO: 68 - protein, 118 aa, Artificial Sequence
  Description of Artificial Sequence Synthetic polypeptide
EVQLVESGGGLVQPGNSLTLSCAASGFTFSKFGMSWVRQANGTSERGLEWVSSISGSGRDTLYADSVKGRFTISRDNAKTTLYLQMNSLRPEDTAVYYCTIGGSLSVSSQGTLVTVSS

SEQ ID NO: 69 - protein, 118 aa, Artificial Sequence
  Description of Artificial Sequence Synthetic polypeptide
EVQLVESGGGLVQPGNSLTLSCAASGFTFSKFGMSWVRQAAAPGSTGLEWVSSISGSGRDTLYADSVKGRFTISRDNAKTTLYLQMNSLRPEDTAVYYCTIGGSLSVSSQGTLVTVSS

SEQ ID NO: 70 - protein, 118 aa, Artificial Sequence
  Description of Artificial Sequence Synthetic polypeptide
EVQLVESGGGLVQPGNSLTLSCAASGFTFSKFGMSWVRQAGKDKKDGLEWVSSISGSGRDTLYADSVKGRFTISRDNAKTTLYLQMNSLRPEDTAVYYCTIGGSLSVSSQGTLVTVSS

SEQ ID NO: 71 - protein, 118 aa, Artificial Sequence
  Description of Artificial Sequence Synthetic polypeptide
EVQLVESGGGLVQPGNSLTLSCAASGFTFSKFGMSWVRQASHGRRQGLEWVSSISGSGRDTLYADSVKGRFTISRDNAKTTLYLQMNSLRPEDTAVYYCTIGGSLSVSSQGTLVTVSS

SEQ ID NO: 72 - protein, 214 aa, Artificial Sequence
  Description of Artificial Sequence Synthetic polypeptide
DIQMTQSPSSVSASVGDRVTITCRASQGIYSWLAWYQQKPGKAPNLLIYTASTLQSGVPSRFSGSGSGTDFTLTISSLQPEDFATYYCQQANIFPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 73 - protein, 456 aa, Artificial Sequence
  Description of Artificial Sequence Synthetic polypeptide
QVQLVQSGAEVKKPGASVKVSCKASGYTFTGYYMHWVRQAPGQGLEWMGWINPDSGGTNYAQKFQGRVTMTRDTSISTAYMELNRLRSDDTAVYYCARDQPLGYCTNGVCSYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 74 - protein, 456 aa, Artificial Sequence
  Description of Artificial Sequence Synthetic polypeptide
QVQLVQSGAEVKKPGASVKVSCKASGYTFTGYYMHWVRQAPGQGLEWMGWINPDSGGTNYAQKFQGRVTMTRDTSISTAYMELNRLRSDDTAVYYCARDQPLGYCTNGVCSYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLDGPSVFLFPPKPKDTLMISRTPEVTCVVVDVEHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 75 - protein, 452 aa, Artificial Sequence
  Description of Artificial Sequence Synthetic polypeptide
QVQLVQSGAEVKKPGASVKVSCKASGYTFTGYYMHWVRQAPGQGLEWMGWINPDSGGTNYAQKFQGRVTMTRDTSISTAYMELNRLRSDDTAVYYCARDQPLGYCTNGVCSYFDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 76 - protein, 452 aa, Artificial Sequence
  Description of Artificial Sequence Synthetic polypeptide
QVQLVQSGAEVKKPGASVKVSCKASGYTFTGYYMHWVRQAPGQGLEWMGWINPDSGGTNYAQKFQGRVTMTRDTSISTAYMELNRLRSDDTAVYYCARDQPLGYCTNGVCSYFDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVAQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 77 - protein, 452 aa, Artificial Sequence
  Description of Artificial Sequence Synthetic polypeptide
QVQLVQSGAEVKKPGASVKVSCKASGYTFTGYYMHWVRQAPGQGLEWMGWINPDSGGTNYAQKFQGRVTMTRDTSISTAYMELNRLRSDDTAVYYCARDQPLGYCTNGVCSYFDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVAQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNAYTQKSLSLSPGK

SEQ ID NO: 78 - protein, 215 aa, Artificial Sequence
  Description of Artificial Sequence Synthetic polypeptide
EIVLTQSPGTLSLSPGERATLSCRASQSVGSSYLAWYQQKPGQAPRLLIYGAFSRATGIPDRFSGSGSGTDFTLTISRLEPEDFAVYYCQQYGSSPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 79 - protein, 448 aa, Artificial Sequence
  Description of Artificial Sequence Synthetic polypeptide
QVQLVESGGGVVQPGRSLRLSCAASGFTFSSYTMHWVRQAPGKGLEWVTFISYDGNNKYYADSVKGRFTISRDNSKNTLYLQMNSLRAEDTAIYYCARTGWLGPFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 80 - protein, 351 aa, Artificial Sequence
  Description of Artificial Sequence Synthetic polypeptide
EVQLVESGGGLVQPGNSLTLSCAASGFTFSKFGMSWVRQARAASAGQGLEWVSSISGSGRDTLYADSVKGRFTISRDNAKTTLYLQMNSLRPEDTAVYYCTIGGSLSVSSQGTLVTVSSGGGGSGGGSGGGGSGGGSDIQMTQSPSSVSASVGDRVTITCRASQGIYSWLAWYQQKPGKAPNLLIYTASTLQSGVPSRFSGSGSGTDFTLTISSLQPEDFATYYCQQANIFPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 81 - protein, 353 aa, Artificial Sequence
  Description of Artificial Sequence Synthetic polypeptide
EVQLVESGGGLVQPGNSLTLSCAASGFTFSKFGMSWVRQAENLRGLRTGLEWVSSISGSGRDTLYADSVKGRFTISRDNAKTTLYLQMNSLRPEDTAVYYCTIGGSLSVSSQGTLVTVSSGGGGSGGGSGGGGSGGGSEIVLTQSPGTLSLSPGERATLSCRASQSVGSSYLAWYQQKPGQAPRLLIYGAFSRATGIPDRFSGSGSGTDFTLTISRLEPEDFAVYYCQQYGSSPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 82 - protein, 353 aa, Artificial Sequence
  Description of Artificial Sequence Synthetic polypeptide
EVQLVESGGGLVQPGNSLTLSCAASGFTFSKFGMSWVRQAPHYAGLITGLEWVSSISGSGRDTLYADSVKGRFTISRDNAKTTLYLQMNSLRPEDTAVYYCTIGGSLSVSSQGTLVTVSSGGGGSGGGSGGGGSGGGSEIVLTQSPGTLSLSPGERATLSCRASQSVGSSYLAWYQQKPGQAPRLLIYGAFSRATGIPDRFSGSGSGTDFTLTISRLEPEDFAVYYCQQYGSSPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 83 - protein, 349 aa, Artificial Sequence
  Description of Artificial Sequence Synthetic polypeptide
EVQLVESGGGLVQPGGSLTLSCAASRFMISEYHMHWVRQAENLRGLRTGLEWVSTINPAGTTDYAESVKGRFTISRDNAKNTLYLQMNSLKPEDTAVYYCDSYGYRGQGTQVTVSSGGGGSGGGSGGGGSGGGSEIVLTQSPGTLSLSPGERATLSCRASQSVGSSYLAWYQQKPGQAPRLLIYGAFSRATGIPDRFSGSGSGTDFTLTISRLEPEDFAVYYCQQYGSSPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 84 - protein, 349 aa, Artificial Sequence
  Description of Artificial Sequence Synthetic polypeptide
EVQLVESGGGLVQPGGSLTLSCAASRFMISEYHMHWVRQAPHYAGLITGLEWVSTINPAGTTDYAESVKGRFTISRDNAKNTLYLQMNSLKPEDTAVYYCDSYGYRGQGTQVTVSSGGGGSGGGSGGGGSGGGSEIVLTQSPGTLSLSPGERATLSCRASQSVGSSYLAWYQQKPGQAPRLLIYGAFSRATGIPDRFSGSGSGTDFTLTISRLEPEDFAVYYCQQYGSSPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 85 - protein, 363 aa, Artificial Sequence
  Description of Artificial Sequence Synthetic polypeptide
QVQLQESGGGLVQAGGSLRLSCAFSGFTFDDFDIGWFRQAPHYAGLITGEREVVSCIASRDGSTYYADSVKGRFTISSDNAKNTVYLQMNSLKPEDTAAYHCAAGKYKTYAVTCPLYDHWGQGTQVTVSSGGGGSGGGSGGGGSGGGSEIVLTQSPGTLSLSPGERATLSCRASQSVGSSYLAWYQQKPGQAPRLLIYGAFSRATGIPDRFSGSGSGTDFTLTISRLEPEDFAVYYCQQYGSSPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 86 - protein, 347 aa, Artificial Sequence
  Description of Artificial Sequence Synthetic polypeptide
EVQLVESGGGLVQPGGSLTLSCAASRFMISEYHMHWVRQARAASAGQGLEWVSTINPAGTTDYAESVKGRFTISRDNAKNTLYLQMNSLKPEDTAVYYCDSYGYRGQGTQVTVSSGGGGSGGGSGGGGSGGGSDIQMTQSPSSVSASVGDRVTITCRASQGIYSWLAWYQQKPGKAPNLLIYTASTLQSGVPSRFSGSGSGTDFTLTISSLQPEDFATYYCQQANIFPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 87 - protein, 361 aa, Artificial Sequence
  Description of Artificial Sequence Synthetic polypeptide
QVQLQESGGGLVQAGGSLRLSCAFSGFTFDDFDIGWFRQARAASAGQGEREVVSCIASRDGSTYYADSVKGRFTISSDNAKNTVYLQMNSLKPEDTAAYHCAAGKYKTYAVTCPLYDHWGQGTQVTVSSGGGGSGGGSGGGGSGGGSDIQMTQSPSSVSASVGDRVTITCRASQGIYSWLAWYQQKPGKAPNLLIYTASTLQSGVPSRFSGSGSGTDFTLTISSLQPEDFATYYCQQANIFPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 88 - protein, 347 aa, Artificial Sequence
  Description of Artificial Sequence Synthetic polypeptide
EVQLVESGGGLVQPGNSLRLSCAASGFTFSKFGMSWVRQAPGKGLEWVSSISGSGRDTLYADSVKGRFTISRDNAKTTLYLQMNSLRPEDTAVYYCTIGGSLSVSSQGTLVTVSSGGGGSGGGSGGGGSGGGSDIQMTQSPSSVSASVGDRVTITCRASQGIYSWLAWYQQKPGKAPNLLIYTASTLQSGVPSRFSGSGSGTDFTLTISSLQPEDFATYYCQQANIFPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 89 - protein, 348 aa, Artificial Sequence
  Description of Artificial Sequence Synthetic polypeptide
EVQLVESGGGLVQPGNSLRLSCAASGFTFSKFGMSWVRQAPGKGLEWVSSISGSGRDTLYADSVKGRFTISRDNAKTTLYLQMNSLRPEDTAVYYCTIGGSLSVSSQGTLVTVSSGGGGSGGGSGGGGSGGGSEIVLTQSPGTLSLSPGERATLSCRASQSVGSSYLAWYQQKPGQAPRLLIYGAFSRATGIPDRFSGSGSGTDFTLTISRLEPEDFAVYYCQQYGSSPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 90 - protein, 350 aa, Artificial Sequence
  Description of Artificial Sequence Synthetic polypeptide
EVQLVESGGGLVQPGNSLTLSCAASGFTFSKFGMSWVRQARAASAGQGLEWVSSISGSGRDTLYADSVKGRFTISRDNAKTTLYLQMNSLRPEDTAVYYCTIGGSLSVSSQGTLVTVSSGGGGPQASTGRSGGGGSDIQMTQSPSSVSASVGDRVTITCRASQGIYSWLAWYQQKPGKAPNLLIYTASTLQSGVPSRFSGSGSGTDFTLTISSLQPEDFATYYCQQANIFPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 91 - protein, 351 aa, Artificial Sequence
  Description of Artificial Sequence Synthetic polypeptide
EVQLVESGGGLVQPGNSLTLSCAASGFTFSKFGMSWVRQAIQPVHTSPGLEWVSSISGSGRDTLYADSVKGRFTISRDNAKTTLYLQMNSLRPEDTAVYYCTIGGSLSVSSQGTLVTVSSGGGGPQASTGRSGGGSEIVLTQSPGTLSLSPGERATLSCRASQSVGSSYLAWYQQKPGQAPRLLIYGAFSRATGIPDRFSGSGSGTDFTLTISRLEPEDFAVYYCQQYGSSPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 92 - protein, 413 aa, Artificial Sequence
  Description of Artificial Sequence Synthetic polypeptide
EPPTACREKQYLINSQCCSLCQPGQKLVSDCTEFTETECLPCGESEFLDTWNRETHCHQHKYCDPNLGLRVQQKGTSETDTICTCEEGWHCTSEACESCVLHRSCSPGFGVKQIATGVSDTICEPCPVGFFSNVSSAFEKCHPWTSCETKDLVVQQAGTNKTDVVCGPQDRLRDYKDDDDKGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 93 - protein, 366 aa, Artificial Sequence
  Description of Artificial Sequence Synthetic polypeptide
AMHVAQPAVVLASSRGIASFVCEYASPGKATEVRVTVLRQADSQVTEVCAATYMMGNELTFLDDSICTGTSSGNQVNLTIQGLRAMDTGLYICKVELMYPPPYYLGIGNGTQIYVIDPEPCPDSDFDYKDDDDKGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 94 - protein, 940 aa, Artificial Sequence
  Description of Artificial Sequence Synthetic polypeptide
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGDYKDDDDKSSNEATNITPKHNMKAFLDELKAENIKKFLYNFTQIPHLAGTEQNFQLAKQIQSQWKEFGLDSVELAHYDVLLSYPNKTHPNYISIINEDGNEIFNTSLFEPPPPGYENVSDIVPPFSAFSPQGMPEGDLVYVNYARTEDFFKLERDMKINCSGKIVIARYGKVFRGNKVKNAQLAGAKGVILYSDPADYFAPGVKSYPDGWNLPGGGVQRGNILNLNGAGDPLTPGYPANEYAYRRGIAEAVGLPSIPVHPIGYYDAQKLLEKMGGSAPPDSSWRGSLKVPYNVGPGFTGNFSTQKVKMHIHSTNEVTRIYNVIGTLRGAVEPDRYVILGGHRDSWVFGGIDPQSGAAVVHEIVRSFGTLKKEGWRPRRTILFASWDAEEFGLLGSTEWAEENSRLLQERGVAYINADSSIEGNYTLRVDCTPLMYSLVHNLTKELKSPDEGFEGKSLYESWTKKSPSPEFSGMPRISKLGSGNDFEVFFQRLGIASGRARYTKNWETNKFSGYPLYHSVYETYELVEKFYDPMFKYHLTVAQVRGGMVFELANSIVLPFDCRDYAVVLRKYADKIYSISMKHPQEMKTYSVSFDSLFSAVKNFTEIASKFSERLQDFDKSNPIVLRMMNDQLMFLERAFIDPLGLPDRPFYRHVIYAPSSHNKYAGESFPGIYDALFDIESKVDPSKAWGEVKRQIYVAAFTVQAAAETLSEVA

SEQ ID NO: 95 - protein, 116 aa, Artificial Sequence
  Description of Artificial Sequence Synthetic polypeptide
EVQLVESGGGLVQPGGSLTLSCAASRFMISEYHMHWVRQAENLRGLRTGLEWVSTINPAGTTDYAESVKGRFTISRDNAKNTLYLQMNSLKPEDTAVYYCDSYGYRGQGTQVTVSS

SEQ ID NO: 96 - protein, 116 aa, Artificial Sequence
  Description of Artificial Sequence Synthetic polypeptide
EVQLVESGGGLVQPGGSLTLSCAASRFMISEYHMHWVRQAPHYAGLITGLEWVSTINPAGTTDYAESVKGRFTISRDNAKNTLYLQMNSLKPEDTAVYYCDSYGYRGQGTQVTVSS

SEQ ID NO: 97 - protein, 115 aa, Artificial Sequence
  Description of Artificial Sequence Synthetic polypeptide
EVQLVESGGGLVQPGGSLTLSCAASRFMISEYHMHWVRQARAASAGQGLEWVSTINPAGTTDYAESVKGRFTISRDNAKNTLYLQMNSLKPEDTAVYYCDSYGYRGQGTQVTVSS

SEQ ID NO: 98 - protein, 130 aa, Artificial Sequence
  Description of Artificial Sequence Synthetic polypeptide
QVQLQESGGGLVQAGGSLRLSCAFSGFTFDDFDIGWFRQAPHYAGLITGEREVVSCIASRDGSTYYADSVKGRFTISSDNAKNTVYLQMNSLKPEDTAAYHCAAGKYKTYAVTCPLYDHWGQGTQVTVSS

SEQ ID NO: 99 - protein, 129 aa, Artificial Sequence
  Description of Artificial Sequence Synthetic polypeptide
QVQLQESGGGLVQAGGSLRLSCAFSGFTFDDFDIGWFRQARAASAGQGEREVVSCIASRDGSTYYADSVKGRFTISSDNAKNTVYLQMNSLKPEDTAAYHCAAGKYKTYAVTCPLYDHWGQGTQVTVSS

SEQ ID NO: 100 - protein, 17 aa, Artificial Sequence
  Description of Artificial Sequence Synthetic peptide
GGGGPQASTGRSGGGGS

SEQ ID NO: 101 - protein, 16 aa, Artificial Sequence
  Description of Artificial Sequence Synthetic peptide
GGGGPQASTGRSGGGS

SEQ ID NO: 102 - protein, 19 aa, Artificial Sequence
  Description of Artificial Sequence Synthetic peptide
GGGGKPLGLQARVVGGGGT

SEQ ID NO: 103 - protein, 8 aa, Unknown
  Description of Unknown Protease cleavage site sequence
GPLGIAGQ

SEQ ID NO: 104 - protein, 8 aa, Unknown
  Description of Unknown Protease cleavage site sequence
AVRWLLTA

SEQ ID NO: 105 - protein, 6 aa, Unknown
  Description of Unknown Protease cleavage site sequence
RRRRRR

SEQ ID NO: 106 - protein, 20 aa, Artificial Sequence
  Description of Artificial Sequence Synthetic peptide
  MISC_FEATURE (1)..(20): This sequence may encompass 1-10 “Gly Ser” repeating units
GSGSGSGSGSGSGSGSGSGS

SEQ ID NO: 107 - protein, 30 aa, Artificial Sequence
  Description of Artificial Sequence Synthetic polypeptide
  MISC_FEATURE (1)..(30): This sequence may encompass 1-10 “Gly Gly Ser” repeating units
GGSGGSGGSGGSGGSGGSGGSGGSGGSGGS

SEQ ID NO: 108 - protein, 40 aa, Artificial Sequence
  Description of Artificial Sequence Synthetic polypeptide
  MISC_FEATURE (1)..(40): This sequence may encompass 1-10 “Gly Gly Gly Ser” repeating units
GGGSGGGSGGGSGGGSGGGSGGGSGGGSGGGSGGGSGGGS

SEQ ID NO: 109 - protein, 40 aa, Artificial Sequence
  Description of Artificial Sequence Synthetic polypeptide
  MISC_FEATURE (1)..(40): This sequence may encompass 1-10 “Gly Gly Ser Gly” repeating units
GGSGGGSGGGSGGGSGGGSGGGSGGGSGGGSGGGSGGGSG

SEQ ID NO: 110 - protein, 50 aa, Artificial Sequence
  Description of Artificial Sequence Synthetic polypeptide
  MISC_FEATURE (1)..(50): This sequence may encompass 1-10 “Gly Gly Ser Gly Gly” repeating units
GGSGGGGSGGGGSGGGGSGGGGSGGGGSGGGGSGGGGSGGGGSGGGGSGG

SEQ ID NO: 111 - protein, 50 aa, Artificial Sequence
  Description of Artificial Sequence Synthetic polypeptide
  MISC_FEATURE (1)..(50): This sequence may encompass 1-10 “Gly Gly Gly Gly Ser” repeating units
GGGGSGGGGSGGGGSGGGGSGGGGSGGGGSGGGGSGGGGSGGGGSGGGGS

SEQ ID NO: 112 - protein, 20 aa, Artificial Sequence
  Description of Artificial Sequence Synthetic peptide
GGGGSGGGGSGGGGSGGGGS

SEQ ID NO: 113 - protein, 15 aa, Artificial Sequence
  Description of Artificial Sequence Synthetic peptide
GGGGSGGGGSGGGGS